(19) United States
(12) Patent Application Publication
(10) Pub. No.: US 2022/0088174 A1
(43) Pub. Date: Mar. 24, 2022

(54) GENOMIC VARIANTS IN IG GENE REGIONS AND USES OF SAME

(71) Applicants: Dana-Farber Cancer Institute, Inc., Boston, MA (US); University of Louisville Research Foundation, Inc., Louisville, KY (US)

(72) Inventors: Wayne A. Marasco, Wellesley, MA (US); Corey Watson, Louisville, KY (US)

(21) Appl. No.: 17/288,684

(22) PCT Filed: Oct. 28, 2019
(86) PCT No.: PCT/US19/58361

§ 371 Date: Apr. 26, 2021

Related U.S. Application Data
(60) Provisional application No. 62/751,256, filed on Oct. 26, 2018.
(60) Provisional application No. 62/775,058, filed on Dec. 4, 2018.

(51) Int. Cl. A61K 39/145; C12Q 1/6883

(52) U.S. Cl. CPC A61K 39/145; C12Q 2600/156; C12Q 2600/106; C12Q 1/6883

(57) ABSTRACT

The present invention is directed to methods for mining genotype-repertoire-disease associations. Aspects of the disclosure are also drawn to methods of preparing a vaccine composition. For example, the vaccine composition can be specific to a subject or a group of subjects with a genotype responsive to the vaccine composition. Aspects of the disclosure are further drawn towards methods of vaccinating a subject or a population of subjects.

This application claims priority from U.S. Provisional Application No. 62/751,256, filed on Oct. 26, 2018, and U.S. Provisional Application No. 62/775,058, filed on Dec. 4, 2018, the entire contents of each of which are incorporated herein by reference.

### GOVERNMENT INTERESTS

This invention was made with government support under grant nos. U01-AI074518, R56-AI109223, R21-AI142590, R24-AI138963 and R01-AI121285 awarded by the National Institute of Allergy & Infectious Disease of the US National Institutes of Health (NIH). The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE DISCLOSURE

Genetic variation in human populations affects how individuals are able to mount functional antibody responses. Different alleles can encode convergent binding motifs that result in successful Ab responses against specific infections and vaccinations. Given the complexity of the IG loci and the diversity of the antibody repertoire, links between IG polymorphism and antibody repertoire variability have not been thoroughly explored.

### SUMMARY OF THE DISCLOSURE

Aspects of the disclosure are directed towards methods for mining genotype—repertoire—disease associations.

Aspects of the disclosure are also drawn to methods of preparing a vaccine composition. For example, the vaccine composition can be specific to a subject or a group of subjects with a genotype responsive to the vaccine composition.

In embodiments, the method comprises the steps of obtaining a biological sample from the subject; identifying germ-line polymorphisms at a immunoglobulin (IG) loci in the tissue sample; identifying antibody repertoire in the tissue sample; comparing the germ-line polymorphisms to the antibody repertoire to identify the subject as responsive to a vaccine composition; and preparing a vaccine composition specific for the subject.

Aspects of the disclosure are further drawn towards methods of vaccinating a subject or a population of subjects.

In embodiments, the method comprises the steps of obtaining a biological sample from the subject; identifying germ-line polymorphisms at a immunoglobulin (IG) loci in the tissue sample; identifying antibody repertoire in the tissue sample; comparing the germ-line polymorphisms to the antibody repertoire to identify the subject as responsive to a vaccine composition; and administering the vaccine composition to the subject.

Still further, aspects of the disclosure are drawn towards methods of identifying a subject or a population of subjects as responsive to a vaccine composition.

In embodiments, the method comprises the steps of obtaining a biological sample from the subject; identifying germ-line polymorphisms at a immunoglobulin (IG) loci in the tissue sample; comparing the germ-line polymorphisms in the tissue sample to known germ-line polymorphisms, wherein the known germ-line polymorphisms are indicative of responsiveness to the vaccine composition; and identifying the subject as responsive to the vaccine composition if the subject's germ-line polymorphisms are similar to the known germ-line polymorphisms.

Also, aspects of the disclosure are drawn towards methods of vaccine discovery.

In embodiments, the method comprises the steps of, the method comprising the steps of obtaining biological samples from a population of subjects; identifying germ-line polymorphisms at a immunoglobulin (IG) loci in the tissue samples; identifying the antibody repertoire in the tissue samples; comparing the germ-line polymorphisms to the antibody repertoires to identify a population as responsive to a vaccine composition.

In embodiments, the immunoglobulin loci comprises an immunoglobulin heavy chain loci, an immunoglobulin light chain loci, or both. For example, the IGH loci comprises the IGHD, IGHC, IGHV, or a combination thereof. In embodiments, the IGH loci comprises the IGHV1-69 loci. For example, the immunoglobulin light chain loci comprises IG lambda, IG kappa, or both.

Embodiments can further comprise the step of evaluating and comparing antibody convergence groups.

Also, embodiments can further comprise the step of administering the vaccine composition to the population of subjects.

In embodiments, the vaccine composition comprise a vaccine composition against an infectious agent, such as an anti-influenza vaccine composition. In embodiments, the vaccine composition can protect against an infection-associated cancer.

In embodiments, identifying germ-line polymorphisms can comprise long-read sequencing of genomic DNA isolated from the biological sample.

In embodiments, identifying the antibody repertoire comprises sequencing cDNA generated from the tissue sample.

In embodiments, the antibody repertoire comprises a naïve antibody repertoire or a stimulated antibody repertoire.

### BRIEF DESCRIPTION OF THE FIGURES

FIG. 1 shows the Basic Overview of Key Elements That Contribute to the Diversity of Naive and Memory Repertoires. A basic schematic of the germ-line IGH locus is shown (not to scale), consisting of clusters of tandemly arrayed IGH V, D, J, and constant (C) gene segments. For a subset of these segments, multiple alleles are shown, representing population-level ‘allelic diversity’ (see Table 1). During the initial formation of the naïve repertoire, single IGH V, D and J gene segment on one of two chromosomes in a given B cell are somatically recombined; at each of these steps, P and N nucleotides are added at the D-J and V-D junctions (‘junctional diversity’), respectively. This process, known as V(D)J rearrangement, is the basis for ‘combinatorial diversity’. The recombined V (red), D (orange), and J (maroon) segments will then be transcribed, and following splicing, will be paired with a C gene (gray). The somatic recombination process also occurs at one of two loci encoding the antibody (Ab) light-chain gene segments [IGK and IGL; except it involves only V (yellow), J (maroon), and C (light gray) gene segments]. Two identical heavy chains and two identical light chains are ultimately paired through disulfide bonds to form a functional Ab; thus, additional diversity in the expressed Ab repertoire comes from ‘heavy- and light-chain pairing’. Together, the V, D, and J segments depicted comprise the variable domain of the heavy chain of a functional antibody, and together with the variable domain of the light chain, encoded by V and J segments, are responsible for antigen (Ag) binding. The C domains of both heavy and light chains provide structural and/or effector functions of the Ab. As shown here for the heavy chain, the variable domain is partitioned into four framework regions (FRs) and three complementarity-determining regions (CDRs). Following Ag stimulation, ‘somatic hypermutations’ introduce additional variation in the variable domain of the Ab (vertical purple bars), with the aim of improving binding affinity. Mutations that arise via SHM can occur across all FRs and CDRs, but these are most prevalent in CDRs, as illustrated by the frequency histogram shown between the unmutated and mutated IG heavy-chain RNA. While the general molecular mechanisms outlined here have long been realized as the primary determinants of diversity within a given expressed Ab repertoire, there is a growing appreciation for the contribution of ‘allelic diversity’ as well, particularly as this pertains to repertoire differences observed between unrelated individuals. Ab, antibody; C, constant; D, diversity; IGH, immunoglobulin heavy-chain locus; IGK, immunoglobulin kappa; IGL, immunoglobulin lambda; J, joining; SHM, somatic hypermutation; V, variable.

FIG. 2 shows a new Paradigm for Integrating Genotypic Information into the Study of the Ab-Mediated Response in Disease and Clinical Phenotypes. In the paradigm, a population cohort is partitioned into subgroups based on functional genotypes/haplotypes that are directly associated with subgroup-specific signatures in the expressed repertoire and other relevant phenotypes (e.g., Abtiter; clinical outcome) associated with the Ab response to a given antigen/epitope. This partitioning can be used to inform tailored clinical care and treatment (e.g., vaccination regime). Ab, antibody.

FIG. 3 shows the Impacts of IG Germ-Line Polymorphism on Ab Repertoire/Structural Diversity. (FIG. 3A) Examples of associations between IG gene region CNV (V gene 1 insertion/deletion) and SNP (noncoding regulatory variant, A/C) genotypes and V gene usage frequencies in the expressed Ab repertoire. (FIG. 3B) Violin plot showing nonsynonymous polymorphism rates in CDR positions with high (>0.6; ‘high’, blue) or low (<0.25; ‘low’, red) frequency of contact with antigen, as labeled on the X axis. The Y axis records, for each CDR-H1 and CDR-H2 position, the number of IMGT IGHV genes that have alleles with nonsynonymous polymorphisms at that position. The positional probability of antigen contact was calculated for each CDR position as the percentage of 150 crystal structures of antibody-antigen complexes from the protein database (PDB) where any atom of that residue is within 5 Å of any antigen atom. Allelic variation is enriched in antigen-contact sites, in that the number of IGHV genes with alleles containing nonsynonymous polymorphisms is greater for high contact probability positions. (FIG. 3C) Genotype frequency differences between five human ethnic groups [Africans (AFR); East Asians (EAS); South Asians (SAS); Central/South American (AMR); and Europeans (EUR)], published by the 1000 Genomes Project [80]*, at two SNPs in IGHV1-69 that have been shown to encode functional residues critical for neutralizing Abs against the influenza HA stem (F54 and L54 amino acid-associated alleles; SNP rs55891010; left panel), and ‘NEAT2’ domain of Staphylococcus aureus (R50 and G50 alleles; SNP rs11845244; right panel). In the left panel, the F allele encodes the functional critical phenylalanine residue, and in the right panel, the primary glycine residue is encoded by the G allele. Interestingly, in both cases, the frequency of individuals lacking alleles encoding the critical residues varies among populations, with the L/L and R/R genotypes showing the lowest frequencies in Africans, and the highest frequencies in South Asians. rs55891010 and rs11845244 are in linkage disequilibrium, and thus R50 and L54 amino acids (and likewise, G50 and F54) tend to co-occur in alleles of IGHV1-69. This explains similarities in genotype frequency estimates between the two SNPs in each population. *Although these genotypes may contain error due to confounds of unrepresented CNV information, they can provide insight into population differences. Ab, antibody; CDR, complementarity-determining region; CNV, copy number variation; HA, hemagglutinin; IG, immunoglobulin; IMGT, ImMunoGeneTics information system database; SNP, single nucleotide polymorphism.

FIG. 4 is a schematic showing that IG are diverse and able to recognize a broad range of pathogens.

FIG. 5 is a schematic showing the linking genetics to antibody expression/function and disease. Antibody repertoire features are strongly correlated between monozygotic twins. (Kohsaka et al., 1996; Glanville et al., 2011; Wang et al., 2015; Rubelt et al., 2016)

FIG. 6 shows graphical data relating to linking genetics to antibody expression/function and disease.

FIG. 7 is a map that shows Immunoglobulin loci are complex at the genomic level and highly polymorphic. Green boxes on functional IGHV genes. Red boxes are pseudo IGHV genes. This figure demonstrates the complexity of the region just in terms of the number of genes; there are >100 known IGHV genes, one of the largest genes families in the human species.

FIG. 8 is a map that shows Immunoglobulin loci are complex at the genomic level and highly polymorphic. Green boxes on functional IGHV genes. Red boxes are pseudo IGHV genes. The triangles under each gene signify whether these gene occurs in deletion/insertion polymorphisms.

FIG. 9 depicts using knowledge of population-level sequence variation to build more effective genotyping assays.

FIG. 10 shows using knowledge of population-level sequence variation to build more effective genotyping assays and analysis pipelines.

FIG. 11 is a graph that shows using knowledge of population-level sequence variation to build more effective genotyping assays and analysis pipelines.

FIG. 12 are graphs that depict using knowledge of population-level sequence variation to build more effective genotyping assays and analysis pipelines.

FIG. 13 are graphs that depict using knowledge of population-level sequence variation to build more effective genotyping assays and analysis pipelines.

FIG. 14 shows a schematic of the Analysis on 18 subjects.

FIG. 15 depicts a Manhattan plot showing significant associations of IGH SNPs and Ab titer.

FIG. 16 are Manhattan plots showing significant associations of IGH SNPs and Ab titer.

FIG. 17 is a heat map of the Analysis on 18 subjects. Using first set of samples (n=18), there are 184 SNPs that associate with at least one strain/time point (p<0.0001). These 184 SNPs are shown here in heat map, ordered by position on chromosome. The strains are ordered on the y axis, by strain and day. The color of tiles corresponds to association P values for a given SNP and Strain/Time point, with red indicating lower p values. (the lowest P value is 3.891169e-06, for SNP in IGHV3-23 region and B.Ohio.Victoria_day0 titer). Some SNPs appear to associate strongly with titers for some strains but not others. For example, the IGHV1-45 region has associations mainly to H5 and H7 strains.

FIG. 18 shows analysis from 53 samples (without SVs). Excluding structural variant regions from the dataset, there are 9149 positions in the locus in which at least one sample has an SNV. The numbers of SNVs called in each sample varies (see plot below), and to some extent depends in part on population. For example, the data indicates that African Americans have higher average SNV counts compared to other groups.

FIG. 19 shows analysis from samples (without SVs). Heterozygosity can be examined among individuals. This can vary by individual and, in part, ethnicity. Heterozygosity can also be plotted for every SNV position across the locus to get a snapshot of IGH-locus patterns of diversity.

FIG. 20 shows analysis from 54 samples (with SVs). With structural variant regions included in the dataset, there are 17864 positions in the locus in which at least one sample has an SNV. The numbers of SNVs called in each sample varies (see bottom plot).

FIG. 21 shows analysis from samples (with SVs). Heterozygosity can also be examined among individuals. This can vary by individual and, in part, ethnicity. Heterozygosity can also be plotted for every SNV position across the locus to get a snapshot of IGH-locus patterns of diversity.

FIG. 22 shows data from 17864 SNPs which were filtered, requiring there to be at least 40 samples with collected data (e.g., not=“NA”), and that among these samples there was at least one het or one homozygous alt genotype (this filter criteria needs to be refined, to better account for how we handle variation within CNVs/SVs). This amounted to a dataset of 11000 SNPs. We used this SNP genotype callset to conduct a linear regression analysis to test for associations between every SNP in the dataset and IgM (time point A) IGHV gene usage (n=48 IGHV genes). We included “Ethnicity” as a covariable. Data described herein focus on genomic variants that “associate” with the usage of IGHV1-69, IGHV3-66, IGHV4-59, and IGHV3-30. Here, manhattan plots show −log 10 pvalues for associations between all SNPs in the callset and time point A IgM gene usage frequency for the four genes mentioned above. Each of these four genes appear to be associated with SNPs in the same region. This region spans the IGHV1-69 and IGHV1-69D region.

FIG. 23 shows the top SNP (1027463) associated with IGHV1-69 IgM time point A gene usage, as well as how it associates with usage of the other three genes. In the case of IGHV1-69, the “ref/ref” genotype is associated with the highest usage frequency, whereas in IGHV3-66, IGHV3-30, and IGHV4-59, that genotype is associated with the lowest usage.

FIG. 24 shows plotting usage of each gene based on combined genotypes of top SNP genotype from manhattan plot (1027463, red circle previous slide) and the taqman based F/L genotypes in the same samples. Without wishing to be bound by theory, this indicates that there is a combinatorial effect (modest) between these two variants.

FIG. 25 shows effects if we also look at usage of these genes in the IgG repertoires at time point B.

FIG. 26 shows testing of our new IGH-capture assay results is ample coverage of the locus and genotyping of locus-wide variants, including CNV, as well as germline coding and non-coding variants. (A) Read depth profiles across the entire IGHJ, D, and V regions, for one haploid (CHM1) and three diploid samples (1, 2, & 3). Red boxes highlight loci in which we previously described large insertions and deletions; these also show read depth variability in across all samples. (B) Read depth profiles for Sample 1 covering functional/ORF IGHV, D, and J genes (left panel). Genotyped IGHV genes in each of the samples at allelic resolution; new alleles are indicated by dots (right panel). (C) An example demonstrating the partitioning of PacBio long-reads spanning the IGHV4-28 gene and 1 Kb flanking regions. In the image, reads in each diploid sample are partitioned into “blue” and “green” clusters, based on the presence of alleles at 4 SNPs in the region. Clusters of haplotype-specific reads can then be assembled to call SNPs and germline alleles, as shown in (B, right panel).

FIG. 27 shows Examples of IGHV1-69 (A,B) and IGHV3-23 (C, D) genotype effects on features of expressed repertoires of 60 healthy adults in cohort 1. Panels (A) and (B) show replication of our previously published findings, demonstrating association (linear regression) between a IGHV1-69 coding variant (SNP, rs55891010), germline gene copy number, and IGHV1-69 gene usage in both IgM (A) and IgG (B). Panels (C) and (D) reveal a gene interaction effect (ANOVA) between the same IGHV1-69 coding variant shown in (A, B) and IGHV3-23 gene copy number; considered in combination, these germline variants contribute to variation observed in IgM (C) and IgG (D) IGHV3-23 gene usage.

FIG. 28 shows immunogenetic characterization of Heavy Chain (VH) Germline Gene Usage for Human Broadly Neutralizing Antibodies Directed Against the Influenza A HA Stem.

FIG. 29 shows Dana-Farber Cancer Institute Cohorts.

FIG. 30 shows (A) the positions of six insertions and three deletions characterized from the CH17 haplotype and fosmid clone resources are shown mapped to GRCh37 in the human IGHV gene region (black line; chr14: 106395611-107289540). Three additional CNVs occur within the red dashed box. but are not depicted. IGHV genes are depicted as green chevrons (not to scale), and segmental duplications are shown below GRCh37, depicted as gray bars. (B) A pairwise BLAST between CH17 and GRCh37 IGHV gene region haplotypes (chr14: 106324366-107268434). Red arrows indicate the positions of CNVs described from Q-117. (C) A miropeats image comparing CH17 to GRCh37 in the region surrounding IGHV1-69. Colored bars represent ˜38 Kbp segmental duplications containing IGHV1-69, found twice in CH17 and only once in GRCh37. (D) Six haplotypes harboring diverse CNVs, including GRCh37 and those described from CH17 and fosmid clones are shown (see “CNV hotspot” in panel A). IGHV genes (green chevrons) and four-25 Kbp segmental duplications (blue bars) exhibiting >94% sequence similarity are shown, and deletions relative to Hapl are depicted as red dotted lines.

FIG. 31 shows (A) application of fosmid-tiling Pacbio assembly in ABC7 identifies a new deletion (lower blue box) that deletes six IGHD genes. This deletion occurs in a complex tandemly duplicated interval on the genome (lower orange box). (B-D) Targeted sequencing of the highly polymorphic IGH locus. (B) The IGHV1⋅69 region contains two known structural haplotypes, one containing a single copy of IGHV1⋅69 and IGHV2⋅70 (top, blue bar), and a second harboring a ˜38 kb duplication of this segment (bottom). As a result, different individuals can carry between 0-4 copies of the IGHV1-69 51p1 allele that encodes HA-stem directed bNAbs. (C) Genotyping of the IGHV1-69 duplication in two individuals using targeted sequencing. Plots show depth of coverage across IGHV1-69 CNV region after mapping to both the CH17 (top, hg38) and hg19 reference assembly (bottom). Read depths for each sample reveal the presence of the single-copy haplotype in Sample 1 and the duplication haplotype in Sample 2. (D) A new protocol that can capture fragments >6 Kb in length was applied to sample 2. Fragments were sequenced with PacBio long-reads with greater ability to reliably reconstruct large structurally variant haplotypes, including duplications. Here, reads are shown overlapping the duplicated IGHV1-69 locus, identified by subtle SNV and deletion patterns that partition each copy.

FIG. 32 shows frequency of IGHV1-69 derived Ab clones in the IgM unmutated (naïve) repertoire of 18 individuals, partitioned by IGHV1-69 genotype (a) and copy number (b). The same significant trends were also observed in IgG memory repertoires. (c) IGHV1-69 allelic variation was also associated with variation in serum blocking post-H5N1 vaccination for binding to H1CA0709, using the hemagglutinin anti-stem F10 broadly-neutralizing Ab. (d) The frequencies of IGHV1-69 alleles and copy numbers vary considerably between human populations. Error bars represent standard error of mean. This work was recently published, Avnir et al., 2016.

FIG. 33 shows IGHV1-69 polymorphism has long range repertoire effects on other IGHV genes in the locus. The usage frequency of IGHV genes over 200 Kb away in the IGH locus also associate with IGHV1-69 allelic genotypes (red, L/L; green, F/L; blue. F/F) and IGHV1-69 repertoire frequency. This was observed in both the unmutated IgM (naive; left) and IgG memory subsets (right). This work was recently published, Avnir et al., 2016.

FIG. 34 shows Population Reference Graph (PRG) construction and sample calling. Construction of the initial PRG (Aim 1.1 3) occurs by (a) alignment of initial reference and fosmid/trio haplotypes and (b) simplifying shared intervals as edges. Samples are identified (c) by identifying diagnostic k-mers in the PRG, (d) selecting a set of paths through the PRG, (e) remapping raw reads to these seed paths, and (I) recalling/refining the haplotype sequence and predicted alleles. (Note a-f adapted from Dilthey et al.) (g) A zoom in of our initial PRG for the IGH locus, constructed from GRC37 and the CH17 (Watson et al.) haplotypes. “Bubbles” in the graph correspond to large SVs/CNVs, other colors correspond to SNVs/indels.

FIG. 35 shows a schematic of Ab repertoire analysis pipeline.(a),the Drop-Seq microfluidic device is used to create water-in-oil single-cell emulsions of B-cells with a cell lysis and poly(dT) bead mixture. (b), B cells are lysed in each droplet and mRNA is captured by a few dozen poly(dT) beads. (c), poly(dT) beads are magnetically recovered and purified. (d), each bead is re-emulsified into individual droplets with RT-PCR mixture. (e), mRNA is reverse transcribed, and overlap-extension (OE) PCR links heavy and light chains into a scFv with cloning sites (f), the scFv library will be analyzed using illumina miseq 2×300 paired end Next-Gen sequencing platform. (g), immunogenetic studies will be conducted using sequencing data to study changes in genotype and expression. (h), the scFv library will also be entered into a yeast surface display pipeline to discover functional nodes against influenza hema99lutinin (HA. (i), yeast clones with functional nodes will be entered into kinetic assays such as ELISA. These assays will include hemagglutinin from various strains of influenza and test for broadly neutralizing capabilities. j) scFv-Fc and IgG1 mAbs can be expressed using mammalian cells and prepared for downstream studies, such as animal trials and structural characterization.

FIG. 36 shows a schematic of an embodiment of the invention.

FIG. 37 shows benchmarking capture and IGenotyper on CHM1. (A) The percentage of IGH with minimum CCS coverage. 98.3% of IGH is spanned by >20 CCS reads (dotted line). The median CCS coverage across IGH genes was 42.5× (inner bottom left plot). (B) CHM1 IGenotyper aligned to itself (GRCh38) shows almost complete coverage. Yellow lines are repetitive alignments >100 bases. (C) Genotypes of IGHJ, D and V genes detected by IGenotyper compared to genotypes in GRCh38. (D) Comparing SNVs detected by IGenotyper and GATK using Illumina data aligned to GRCh37 to ground truth CHM1 SNVs detected by aligning the IGH locus in GRCh38 to GRCh37.

FIG. 38 show number of variants found across sample and validation of variants in NA19240 and NA12878. (A) Different variants types (labeled by different colors) are found in sequence features of the IGH locus across all the samples. (B) SNVs, indels and SVs were validated by checking the presence of variants within the parents.

FIG. 39 shows large improvement in SNV detection with consequential implications. (A) SNVs detected with short read data and with IGenotyper in CHM1 were compared to a ground truth SNV dataset. IGenotyper encompassed almost all true SNVs and detected very few false SNVs. SNVs detected with short read data contained a large amount of false SNVs and missed many true SNVs. (B) A large amount of SNVs within the 1000 Genomes Phase 3 SNVs call sets in NA12878 and NA19240 are false. SNVs found by IGenotyper in NA19240 and NA12878 as well as in the parents (purple circle) were not present in the 1000 Genomes Phase 3 SNVs call set. (C) The 1000 Genomes Phase 3 SNVs call sets is used for imputing SNVs detected for chip arrays. Half of the imputed SNVs from Park et al were incorrect and 2,562 SNVs were missed.

FIG. 40 is a schematic that shows immunoglobulin loci are complex at the genomic level and highly polymorphic.

### DETAILED DESCRIPTION OF THE INVENTION

Antibodies (Abs) produced by immunoglobulin (IG) genes are the most diverse proteins expressed in humans. While part of this diversity is generated by recombination during B-cell development and mutations during affinity maturation, the germ-line IG loci are also diverse across human populations and ethnicities. Recently, proof-of-concept studies have demonstrated genotype-phenotype correlations between specific IG germ-line variants and the quality of Ab responses during vaccination and disease. However, the functional consequences of IG genetic variation in Ab function and immunological outcomes remain underexplored. Interconnections between IG genomic diversity and Ab-expressed repertoires and structure are presented. The inventors further detail a strategy for integrating IG genotyping with functional Ab profiling data as a means to better assess and optimize humoral responses in genetically diverse human populations, with immediate implications for personalized medicine. For example, such strategies can comprise methods of preparing a vaccine composition, methods of vaccine discovery, or methods of identifying a subject as responsive to a particular vaccine composition. Thus, various exemplary embodiments of the present disclosure comprise methods for mining genotype—repertoire—disease associations.

Detailed descriptions of one or more embodiments are provided herein. However, the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a,” “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be non-limiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises,” “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Vaccine Composition

Aspects of the disclosure are drawn to vaccine compositions that are discovered and/or prepared by methods described herein. For example, the discovery of such vaccine compositions can be based on genotype-phenotype correlations between specific IG germ-line variants and the quality of Ab responses during vaccination and disease.

The terms “vaccine” or “vaccine composition”, which can be used interchangeably, can refer to pharmaceutical compositions containing at least one immunogenic composition that induces an immune response in a subject, such as a human. The vaccine or vaccine composition can protect the subject from disease or death, such as due to infection or cancer. Such vaccine compositions can optionally include may or may not include one or more additional components that enhance the immunological activity of the active component. The vaccine or vaccine composition can further comprise additional components typical of pharmaceutical compositions. The vaccine or vaccine composition can further comprise additional components typical of vaccines or vaccine compositions, including but not limited to, for example, an adjuvant or immunomodulator.

The immunogenically active component of the vaccine can comprise a peptide, which can be referred to as a “peptide-based vaccine”, “peptide vaccine”, or “antigenic polypeptide”.

For example, an “antigenic polypeptide” or an “immunogenic polypeptide” can refer to a polypeptide which, when introduced into a vertebrate, reacts with the vertebrate's immune system molecules, i.e., is antigenic, and/or induces an immune response in the vertebrate, i.e., is immunogenic. Examples of antigenic and immunogenic polypeptides include, but are not limited to, e.g., HA or fragments or variants thereof. Isolated antigenic and immunogenic polypeptides can be provided as a recombinant protein, a purified subunit, a viral vector expressing the protein, or can be provided in the form of an inactivated virus vaccine, e.g., a live-attenuated virus vaccine, a heat-killed virus vaccine, etc.

Antigenic polypeptides can be produced using any techniques available to those of ordinary skill in the art, such as chemical and biochemical synthesis. Examples of techniques for chemical synthesis of peptides are provided in Lee, Peptide and Protein Drug Delivery, New York, N.Y., Dekker (1990); in Ausubel, Current Protocols in Molecular Biology, John Wiley, 1987-1998, and in Sambrook et al. (1989); each of which is also specifically incorporated herein in its entirety by express reference thereto.

A “recombinant protein vaccine” can refer to a vaccine whose active ingredient includes at least one protein antigen that is produced by recombinant expression. The vaccine antigens can be produced in bacteria, mammalian cells, baculovirus cells, and/or plant cells, or hybrids thereof, for example. An exemplary method of producing influenza vaccines involves growth of an isolated strain in embryonated hen's eggs.

Preparation of peptide-based vaccines is generally well understood by those of ordinary skill in the art, and can be accomplished by a variety of available techniques, including, for example, those described in U.S. Pat. Nos. 4,608,251; 4,601,903; 4,599,231; 4,599,230; and 4,596,792; and generally as provided in Remington's Pharmaceutical Sciences, 16th Edition, A. Osol, (ed.), Mack Publishing Co., Easton, Pa. (1980), and Remington's Pharmaceutical Sciences, 19th Edition, A. R. Gennaro, (ed.), Mack Publishing Co., Easton, Pa. (1995), each of which is specifically incorporated herein in its entirety.

The immunogenically active component of the vaccine can contain whole living organisms either in their original form or in the form of attenuated organisms in a modified live vaccine, or organisms inactivated by suitable methods in a killed or inactivated vaccine, or subunit vaccines containing one or more immunogenic components of the virus, or genetically engineered, mutated or cloned vaccines obtained by methods known to those skilled in the art. A vaccine may contain one or more than one of the elements described above. For example, vaccine compositions can include, but are not limited to, live, attenuated, or killed/inactivated forms of whole influenza virus, infectious nucleic acids encoding influenza virus, or other infectious DNA vaccines, including plasmids, vectors, or other carriers for direct DNA injection.

The term “antigen” or “immunogen” can refer to a substance that induces a specific immune response in a subject. The antigen can comprise a whole organism, killed, attenuated or live; a subunit or portion of an organism; a recombinant vector containing an insert with immunogenic properties, such as a peptide vaccine produced by recombinant methods; a piece or fragment of DNA capable of inducing an immune response upon presentation to a host animal; a protein, a polypeptide, a peptide, an epitope, a hapten, or any combination thereof. Alternately, the immunogen or antigen can comprise a toxin or antitoxin. An antigen generally encompasses any immunogenic substance, i.e., any substance that elicits an immune response (e.g., the production of specific antibody molecules) when introduced into the tissues of a susceptible subject, and that is capable of specifically binding to an antibody that is produced in response to the introduction of the antigen. An antigen is capable of being recognized by the immune system, inducing a humoral immune response, and/or inducing a cellular immune response leading to the activation of B- and/or T-lymphocytes. An antigen may include a single epitopes, or include two or more epitopes. An antigen may include one or more native or synthetic immunogenic components, and may optionally be administered in, or with, one or more adjuvants.

The term “antibody” can refer to a protein that binds to other molecules (can be referred to as antigens) via heavy and light chain variable domains, VH and VL, respectively. The term “antibody” can refer to any immunoglobulin molecule, including, for example, but not limited to, IgM, IgG, IgA, IgE, IgD, and any subclass thereof or combination thereof. The term “antibody” can also refer to a functional fragment of immunoglobulin molecules, including for example, but not limited to, Fab, Fab′, (Fab′)2, Fv, Fd, scFv and sdFv fragments unless otherwise expressly stated. For example, the term “HA antibody” or “anti-HA antibody,” as used herein, means an antibody that specifically binds to an a hemagglutinin protein or a portion (epitope) thereof.

Vaccine compositions described herein can formulated to be compatible with its intended route of administration. Examples of routes of administration include parenteral, e.g., intravenous, intradermal, subcutaneous, oral, nasal, transdermal (topical), transmucosal, and rectal administration. Solutions or suspensions can include the following components: a sterile diluent such as water, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The preparation can be enclosed in ampoules, disposable syringes or multiple dose vials made of glass or plastic.

The vaccine composition can comprise a pharmaceutically acceptable carrier. The term “carrier” can include any solvent(s), dispersion medium, coating(s), diluent(s), buffer(s), isotonic agent(s), solution(s), suspension(s), colloid(s), inert(s) or such like, or a combination thereof. The use of one or more delivery vehicles for chemical compounds in general, and peptides and epitopes in particular, is well known to those of ordinary skill in the pharmaceutical arts. Except insofar as any conventional media or agent is incompatible with the active ingredient, its use in the therapeutic compositions is contemplated. One or more supplementary active ingredient(s) can also be incorporated into one or more of the disclosed immunogenic compositions.

Aspects of the disclosure are drawn to the identification and preparation of vaccine compositions for a specific subject or population of subjects. Such methods can be considered a personalized approach to vaccine the subject or population of subjects to protect against a disease, such as an infection or cancer. As discussed in more detail elsewhere herein, the methods typically comprise the steps of obtaining or isolating a biological sample from a subject, and/or isolating or obtaining genomic DNA or mRNA from a biological sample from a subject; identifying germ-line polymorphisms at an IG loci, such as the immunoglobulin heavy chain (IGH) loci and/or the immunoglobulin light chain (IGL) loci; identifying antibody repertoire in the biological sample; comparing and, optionally, contrasting the germ-line polymorphisms to the antibody repertoire to identify the subject as responsive to a particular vaccine composition; and preparing the vaccine composition specific for the subject.

Aspects of the disclosure are also drawn to vaccine compositions discovered by methods described elsewhere herein. For example, the methods can typically comprise the steps of obtaining or isolating biological samples from a population of subjects, and/or isolating or obtaining genomic DNA or mRNA from biological samples from a population of subjects; identifying germ-line polymorphisms at an IG loci, such as the immunoglobulin heavy chain (IGH) loci and/or the immunoglobulin light chain (IGL) loci; identifying antibody repertoire in the biological samples; comparing and contrasting the germ-line polymorphisms to the antibody repertoires to identify the population as responsive to a particular vaccine composition. The methods can further comprise the step of preparing the vaccine composition specific for the population of subjects.

As described herein the vaccine composition can be administered to a subject in a “therapeutically effective amount” or “immunogenically effective amount. The term “therapeutically effective amount” can refer to those amounts of the vaccine composition that, when administered to a particular subject in view of the nature and severity of that subject's disease or condition, will have a desired therapeutic effect, e.g., an amount which will cure, prevent, inhibit, or at least partially arrest or partially prevent a target disease or condition. In some embodiments, the term “therapeutically effective amount” or “effective amount” can refer to an amount of a therapeutic agent that when administered alone or in combination with an additional therapeutic agent to a cell, tissue, or subject is effective to prevent or ameliorate the disease or condition. A therapeutically effective dose further refers to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. A therapeutically effective dose can depend upon a number of factors known to those of ordinary skill in the art. The dose(s) can vary, for example, depending upon the identity, size, and condition of the subject or sample being treated, further depending upon the route by which the composition is to be administered, if applicable, and the effect which the practitioner desires. These amounts can be readily determined by the skilled artisan

The term “immunogenically-effective amount” can refer to an amount of an immunogen that is capable of inducing an immune response that significantly engages pathogenic agents that share immunological features with the immunogen. This term can also encompass either therapeutic or prophylactic effective amounts, or both.

Method of Preparing a Vaccine Composition

Aspects of the disclosure are drawn to various methods that leverage genotype—antibody repertoire—disease associations for human health. For example, embodiments can comprise methods of preparing vaccine compositions specific to a subject or a population of subjects with a genotype(s) responsive to the vaccine composition. Other embodiments can comprise methods of vaccine discovery. Generally, the methods comprise the steps of obtaining or isolating a biological sample from a subject or from a population of subjects, and optionally isolating genomic DNA and/or mRNA from the biological sample; identifying germ-line polymorphisms at an IG loci, such as the immunoglobulin heavy chain (IGH) loci and/or the immunoglobulin light chain (IGL) loci; identifying antibody repertoire in the biological sample(s); comparing and, optionally, contrasting the germ-line polymorphisms to the antibody repertoires to identify the subject or population as responsive to a particular vaccine composition. The methods can further comprise the step of preparing the vaccine composition specific for the subject or population of subjects. The method can further comprise the step of administering the vaccine composition to the subject or population of subjects.

Aspects of the disclosure are further drawn to methods of determining IG genotypes for one or more subjects. The term “genotype” with respect to a particular gene refers to a sum of the alleles of the gene contained in an individual or a sample. The phrase “determining the genotype” of an IG gene can refer to determining the polymorphisms present in the individual alleles of the IG gene present in a subject.

In embodiments, the method can comprise, for each individual, performing an amplification reaction with a forward primer and a reverse primer, each primer comprising an adapter sequence, an individual identification sequence, and a IG-hybridizing sequence, to amplify the exon sequences of the IG genes that comprise polymorphic sites to obtain IG amplicons; pooling IG amplicons from more than one individual obtained in the first step; performing emulsion PCR; determining the sequence of each IG amplicon for each individual using pyrosequencing in parallel; and assigning the IG alleles to each individual by comparing the sequence of the IG amplicons determined in the previous step to known IG sequences to determine which IG alleles are present in the individual.

The term “allele” can refer to a sequence variant of a gene. At least one genetic difference can constitute an allele. For IG genes, multiple genetic differences typically constitute an allele. The term “haplotype” can refer to a combination of alleles at different places (loci or genes) on the same chromosome in an individual.

The term “amplicon” can refer to a nucleic acid molecule that contains all or fragment of the target nucleic acid sequence and that is formed as the product of in vitro amplification by any suitable amplification method. The IG amplicons can be obtained using any type of amplification reaction. For example, the IG amplicons are typically made by PCR using primer pairs.

In embodiments, the genotypes of the one or more subject can be determined in parallel.

As described herein, embodiments can comprise steps of obtaining or isolating a biological sample from a subject or from a population of subjects. The phrase “biological sample” or “tissue sample”” can refer to a sample of biological material obtained from or isolated from a subject, such as a human subject. The sample can be obtained by any means known to those of skill in the art. Such sample can be an amount of tissue or fluid, or a purified fraction thereof, isolated from an individual or individuals, including tissue or fluid, for example, skin, plasma, serum, whole blood and blood components, spinal fluid, saliva, peritoneal fluid, lymphatic fluid, aqueous or vitreous humor, synovial fluid, urine, tears, seminal fluid, vaginal fluids, pulmonary effusion, serosal fluid, organs, bronchio-alveolar lavage, tumors and paraffin embedded tissues. Samples also may include constituents and components of in vitro cultures of cells obtained from an individual, including, but not limited to, conditioned medium resulting from the growth of cells in the cell culture medium, recombinant cells and cell components. Other non-limiting examples of samples include a tissue, a tissue sample, a cell sample (e.g., a tissue biopsy, such as, an aspiration biopsy, a brush biopsy, a surface biopsy, a needle biopsy, a punch biopsy, an excision biopsy, an open biopsy, an incision biopsy or an endoscopic biopsy), a tumor sample, or a sample of a biological fluid (e.g., blood, ascites, serum, saliva, urine, nipple aspirates). For example, a “tissue sample” can refer to a portion, piece, part, segment, or fraction of a tissue which is obtained or removed from an intact tissue of a subject, preferably a human subject.

The phrase “obtaining a biological sample” can refer to any process for directly or indirectly acquiring a biological sample from a subject. For example, a biological sample can be obtained (e.g., at a point-of-care facility, e.g., a physician's office, a hospital, laboratory facility) by procuring a tissue or fluid sample (e.g., blood draw, marrow sample, spinal tap) from a subject. Alternatively, a biological sample may be obtained by receiving the biological sample (e.g., at a laboratory facility) from one or more persons who procured the sample directly from the subject. The biological sample may be, for example, a tissue (e.g., blood), cell (e.g., hematopoietic cell such as hematopoietic stem cell, leukocyte, or reticulocyte, stem cell, or plasma cell), vesicle, biomolecular aggregate or platelet from the subject.

Embodiments of the disclosure can also utilize isolates of a biological sample in the methods of the invention. As used herein, an “isolate” of a biological sample (e.g., an isolate of a tissue or tumor sample, or of a biological fluid) can refer to a material or composition (e.g., a biological material or composition) which has been separated, derived, extracted, purified or isolated from the sample and preferably is substantially free of undesirable compositions and/or impurities or contaminants associated with the biological sample. For example, the phrase “substantially free” or “substantially purified” can refer to recovery of a material or composition which is at least 80% and preferably 90-95% purified with respect to removal of a contaminant. For example, the isolation of a nucleic acid (e.g., DNA or mRNA) that is substantially purified can be free of contaminants such as cellular components (e.g., protein, lipid or salt). Thus, the term “substantially purified” can generally refer to separation of a majority of cellular proteins or reaction contaminants from the sample, so that compounds capable of interfering with the subsequent use of the isolated nucleic acid are removed.

Embodiments can comprise steps of isolating nucleic acids, or obtaining a nucleic acid sample, from a biological sample. For example, embodiments can comprise isolating genomic DNA and/or mRNA from the biological sample

As used herein, the term “nucleic acid sample” can refer to a sample comprising nucleic acids. A “nucleic acid” can refer to a DNA, an RNA, modified DNA, modified RNA, and the like. A nucleic may comprise any number of nucleotides, e.g., from 2 to over a million nucleotides. Size may be defined by mass, length, or other suitable size measures. The length of a nucleic acid may be expressed in units indicating as a number of “base pairs” (abbreviated “bp”), a number of “bases”, or a number of nucleotides (“nt” or “nts”). Lengths of double stranded nucleic acids (e.g., DNA) are typically, but not exclusively, expressed in units of base pairs (bp). Lengths of single stranded nucleic acids (e.g., DNA) are typically, but not exclusively, expressed in units of nucleotides (nt). Lengths expressed in units of bases may apply to either double stranded nucleic acids or single stranded nucleic acids. These units are modifiable with standard SI prefixes to indicate multiples of powers of 10, e.g., kbp, Mbp, Gbp, kilobase, Megabase, Gigabase, etc.),

The size measurement can be performed in various ways known in the art, e.g., paired-end sequencing and alignment of nucleic acids, electrophoresis, centrifugation, optical methods, mass spectrometry, etc. A statistically significant number of nucleic acids can be measured to provide an accurate size profile of a sample. In some embodiments, the data obtained from a physical measurement is received at a computer and analyzed to accomplish the measurement of the sizes of the nucleic acids.

In embodiments, a “sample of DNA” or “DNA sample” can refer to a sample comprising DNA or nucleic acid representative of DNA isolated from a natural source and in a form suitable for evaluation by an assay (e.g., as a soluble aqueous solution).

In embodiments, one or more nucleotide polymorphisms (such as germline polymorphisms) are identified. “Nucleotide polymorphism” can refers to the occurrence of two more alternative bases at a defined location that may or may not affect the coding sequence, gene or resulting proteins. The base changes may be a single base change, also known as a “single nucleotide polymorphism” or “SNP” or “snip”. The base changes may be multiple base substitutions of the sequence at the location, and may include insertion and deletion sequence. A polymorphic position can refer to a site in the nucleic acid where the polymorphic nucleotide that distinguishes the variants occurs. A polymorphism can also include large structural variants, such as large insertions or deletions of nucleotide sequence that can contain IG gene segments or regulatory elements.

For example, embodiments can comprise isolating genomic DNA and/or mRNA from the biological sample and identifying germline polymorphisms. The phrase “germline nucleic acid residue” can refer to the nucleic acid residue that naturally occurs in a germline gene, such as a germline gene encoding a constant or variable region. “Germline gene” is the DNA found in a germ cell (i.e., a cell destined to become an egg or in the sperm). A “germline mutation” or “germline polymorphism” thus can refer to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell.

The identification of germline polymorphisms can be completed by, for example, DNA sequencing. DNA sequencing methods are known to the skilled artisan, and include high-throughput sequencing, next-generation sequencing, long-read sequencing. Embodiments, for example, can comprise target-enrichment DNA capture with long-read sequencing See, for example, the protocol published by Pacific BioSciences titled “Target Sequence Capture Using Roche NimbleGen SeqCap EZ Library” (see, for example, https://www.pacb.com/wp-content/uploads/Procedure-Checkli st-%E2%80%93-Multiplex-Genomic-DNA-Target-Capture-Using-SeqCap-EZ-Libraries.pdf, which is incorporated by reference herein in its entirety).

In particular, long-read sequencing technologies can resolve complex regions such as killer immunoglobulin-like receptors (KIR), human leukocyte antigen (HLA) and chromosomal rearrangements, identify novel structural variants (SVs), and identify SVs missed by standard short-read sequencing methods. Additionally, the sensitivity of SV detection can be improved by attempting to resolve variants in a haplotype-specific manner. When long-read sequencing is combined with methods to specifically target a genomic locus, either with a CRISPR/Cas9 system or DNA probes, it can effectively resolve such regions. Targeted approaches have also enabled a higher resolution of HLA typing and KIR typing.

Referring to the Example, germline polymorphisms can be identified by long-read sequencing. For example, long-read sequencing allows from the retrieval of much longer (>10,000 bp, in certain instances) sequencing reads than widely-used short-read sequencing systems (75-300 bp).

In an embodiment, an IGH locus capture assay can be paired with long-read sequencing to characterize haplotype and population diversity in the IG loci. For example, such an assay can use nimblegen SeqCap probes to pull down ˜1.2 Mb of sequence targets in human IGHV/D/J gene regions. This can result in sequencing libraries of 6-8 kb, ideal for leveraging the strengths of long-reads for CNV calling and SNP genotyping. Embodiments can further utilize existing and in-house pipelines, such as BLASR, Quiver, WhatsHap, and MsPAC to map, partition, and assemble reads, for SNP calling, gene/allele assignment, and CNV detection.

The terms “target region” or “target sequence” can refer to a polynucleotide sequence to be studied in a sample. In the context of the present disclosure, the target sequences are the IG gene sequences contained in the biological sample from a subject.

The term “oligonucleotide” can refer to a short nucleic acid, typically ten or more nucleotides in length. Oligonucleotides are prepared by any suitable method known in the art, for example, direct chemical synthesis as described in Narang et al. (1979) Meth. Enzymol. 68:90-99; Brown et al. (1979) Meth. Enzymol. 68:109-151; Beaucage et al. (1981) Tetrahedron Lett. 22:1859-1862; Matteucci et al. (1981) J. Am. Chem. Soc. 103:3185-3191; or any other method known in the art.

The term “primer” can refer to an oligonucleotide, which is capable of acting as a point of initiation of nucleic acid synthesis along a complementary strand of a template nucleic acid. A primer that is at least partially complementary to a subsequence of a template nucleic acid is typically sufficient to hybridize with template nucleic acid and for extension to occur. Although other primer lengths are optionally utilized, primers typically comprise hybridizing regions that range from about 6 to about 100 nucleotides in length and most commonly between 15 and 35 nucleotides in length. The design of suitable primers for the amplification of a given target sequence is well known in the art and described in the literature cited herein. The design of suitable primers for parallel clonal amplification and sequencing is described e.g. in a U.S. Application Pub. No. 20100086914.

A “thermostable nucleic acid polymerase” or “thermostable polymerase” is a polymerase enzyme, which is relatively stable at elevated temperatures when compared, for example, to polymerases from E. coli. As used herein, a thermostable polymerase is suitable for use under temperature cycling conditions typical of the polymerase chain reaction (“PCR”).

The term “adapter region” of a primer can refer to the region of a primer sequence at the 5′ end that is universal to the IG amplicons obtained in the method of the present disclosure and provides sequences that anneal to an oligonucleotide present on a microparticle (i.e. bead) or other solid surface for emulsion PCR. The adapter region can further serve as a site to which a sequencing primer binds. The adapter region is typically from 15 to 30 nucleotides in length.

The terms “library key tag” can refer to the portion of an adapter region within a primer sequence that serves to differentiate a KIR-specific primer from a control primer.

The terms “multiplex identification tag”, “individual identification tag”—or “MID” are used interchangeably and can refer to a nucleotide sequence present in a primer that serves as a marker of the DNA obtained from a particular subject or sample.

The terms “nucleic acid” can refers to polymers of nucleotides (e.g., ribonucleotides and deoxyribonucleotides, both natural and non-natural) such polymers being DNA, RNA, and their subcategories, such as cDNA, mRNA, etc. A nucleic acid may be single-stranded or double-stranded and will generally contain 5′-3′ phosphodiester bonds, although in some cases, nucleotide analogs may have other linkages. Nucleic acids may include naturally occurring bases (adenosine, guanosine, cytosine, uracil and thymidine) as well as non-natural bases. The example of non-natural bases include those described in, e.g., Seela et al. (1999) Helv. Chim. Acta 82:1640. Certain bases used in nucleotide analogs act as melting temperature (Tm) modifiers. For example, some of these include 7-deazapurines (e.g., 7-deazaguanine, 7-deazaadenine, etc.), pyrazolo[3,4-d]pyrimidines, propynyl-dN (e.g., propynyl-dU, propynyl-dC, etc.), and the like. See, e.g., U.S. Pat. No. 5,990,303, which is incorporated herein by reference. Other representative heterocyclic bases include, e.g., hypoxanthine, inosine, xanthine; 8-aza derivatives of 2-aminopurine, 2,6-diaminopurine, 2-amino-6-chloropurine, hypoxanthine, inosine and xanthine; 7-deaza-8-aza derivatives of adenine, guanine, 2-aminopurine, 2,6-diaminopurine, 2-amino-6-chloropurine, hypoxanthine, inosine and xanthine; 6-azacytidine; 5-fluorocytidine; 5-chlorocytidine; 5-iodocytidine; 5-bromocytidine; 5-methylcytidine; 5-propynylcytidine; 5-bromovinyluracil; 5-fluorouracil; 5-chlorouracil; 5-iodouracil; 5-bromouracil; 5-trifluoromethyluracil; 5-methoxymethyluracil; 5-ethynyluracil; 5-propynyluracil, and the like.

The terms “natural nucleotide” refer to purine- and pyrimidine-containing nucleotides naturally found in cellular DNA and RNA: cytosine (C), adenine (A), guanine (G), thymine (T) and uracil (U).

The term “non-natural nucleotide” or “modified nucleotide” can refer to a nucleotide that contains a modified base, sugar or phosphate group, or that incorporates a non-natural moiety in its structure. The non-natural nucleotide can be produced by a chemical modification of the nucleotide either as part of the nucleic acid polymer or prior to the incorporation of the modified nucleotide into the nucleic acid polymer. In another approach a non-natural nucleotide can be produced by incorporating a modified nucleoside triphosphate into the polymer chain during enzymatic or chemical synthesis of the nucleic acid. Examples of non-natural nucleotides include dideoxynucleotides, biotinylated, aminated, deaminated, alkylated, benzylated and fluorophor-labeled nucleotides.

The term “nucleic acid polymerases” or simply “polymerases” can refer to enzymes, for example, DNA polymerases, that catalyze the incorporation of nucleotides into a nucleic acid. Exemplary thermostable DNA polymerases include those from Thermus thermophilus, Thermus caldophilus, Thermus sp. ZO5 (see, e.g., U.S. Pat. No. 5,674,738) and mutants of the Thermus sp. ZO5 polymerase (see, e.g. U.S. patent application Ser. No. 11/873,896, filed on Oct. 17, 2007), Thermus aquaticus, Thermus flavus, Thermus filiformis, Thermus sp. sps17, Deinococcus radiodurans, Hot Spring family B/clone 7, Bacillus stearothermophilus, Bacillus caldotenax, Escherichia coli, Thermotoga maritima, Thermotoga neapolitana and Thermosipho africanus. The full nucleic acid and amino acid sequences for numerous thermostable DNA polymerases are available in the public databases.

The terms “polymerase chain reaction amplification conditions” or “PCR conditions” can refer to conditions under which primers that hybridize to a template nucleic acid are extended by a polymerase during a polymerase chain reaction (PCR). Those of skill in the art will appreciate that such conditions can vary, and are generally influenced by the nature of the primers and the template. Various PCR conditions are described in PCR Strategies (M. A. Innis, D. H. Gelfand, and J. J. Sninsky eds., 1995, Academic Press, San Diego, Calif.) at Chapter 14; PCR Protocols: A Guide to Methods and Applications (M. A. Innis, D. H. Gelfand, J. J. Sninsky, and T. J. White eds., Academic Press, NY, 1990).”

As described herein, embodiments comprise identifying germline mutations (i.e., germline polymorphisms) in an immunoglobulin (IG) loci. Antibodies (Abs) are a diverse family of proteins expressed by B cells and are encoded by hundreds of genes at three primary immunoglobulin (IG) gene regions. Whereas the heavy chain is encoded by genes at the IG heavy-chain locus (IGH), the light chain can be encoded by genes at either the IG kappa (IGK) or IG lambda (IGL) chain loci.

The IGH locus, for example, exhibits extreme genetic variability at both the individual and population levels. This extreme variation is characterized by the occurrence of single nucleotide polymorphisms (SNPs), as well as large insertions, deletions, and duplications spanning tens of thousands of kilobases, and resulting in losses or gains of functional genes (copy number variants, CNVs). The IGH locus consists of approximately 54 V, 23 D, 6 J, and 9 C functional/open reading frame genes that can contribute to the formation of expressed antibodies. Referring to the examples, germline polymorphisms can be identified in the IGHV/D/J gene containing regions of human chromosome 14, for example, using a high-throughput approach.

Embodiments further comprise identifying antibody repertoire in a biological sample. In embodiments, the phrase “antibody repertoire” can refer to the entire set of antibodies produced, such as in reference to a particular subject. For example, antibody repertoire can refer to the sum of each of the different antibody species in an animal or human being. An antibody repertoire can contain multiple antibodies to different proteins, and can also comprise antibodies against different epitopes of the same protein.

The identification of antibody repertoire can be completed by methods known to the skilled artisan. See, for example, DeKosky, Brandon J., et al. “Large-scale sequence and structural comparisons of human naïve and antigen-experienced antibody repertoires.” Proceedings of the National Academy of Sciences 113.19 (2016): E2636-E2645; Bashford-Rogers, Rachael J M, Kenneth G C Smith, and David C. Thomas. “Antibody repertoire analysis in polygenic autoimmune diseases.” Immunology 155.1 (2018): 3-17; and Robinson, William H. “Sequencing the functional antibody repertoire—diagnostic and therapeutic discovery.” Nature Reviews Rheumatology 11.3 (2015): 171. For example, the identification of antibody repertoire can be completed by high throughput sequencing approaches for profiling the expressed antibody repertoire. For example, methods can include sequencing cDNA generated from the biological sample. In embodiments, high resolution descriptions of dynamic features of naïve and antigen-stimulated antibody repertoires can be identified by RepSeq (Repertoire Sequencing).

In embodiments, the antibody repertoire can be naïve antibody repertoires or antigen-stimulated antibody repertoire.

In embodiments, the antibody repertoire can be a human antibody repertoire, or can be an antibody repertoire of a non-human, such as a mouse, a rat, or other animal.

Embodiments also comprise comparing the identified germline polymorphisms to the identified antibody repertoire to identify the subject or population of subjects as response to a vaccine composition. Generally speaking, the term “comparing” can refer to any suitable method of evaluating, calculating or processing data. For example, embodiments described herein can comprise a step of comparing one or more germ-line polymorphisms to an antibody repertoire, or vice versa. In embodiments, for example, the term “comparing” can refer to making an assessment of how the germ-line polymorphisms identified in methods herein relate to the antibody repertoire of a subject identified in methods herein. In certain embodiments, methods can comprise comparing a subject's germ-line polymorphisms, antibody repertoire, or both, to a control sample. For example, the control sample can be germ-line polymorphisms or antibody repertoire of a population of individuals.

Method of Vaccinating a Subject

Aspects of the invention are also drawn to methods of vaccination a subject or a population of subjects. Generally, the methods of vaccination comprise the steps of obtaining or isolating a biological sample from a subject or from a population of subjects, and optionally isolating genomic DNA and/or mRNA from the biological sample; identifying germ-line polymorphisms at an IG loci, such as the immunoglobulin heavy chain (IGH) loci and/or the immunoglobulin light chain loci, such as immunoglobulin lamda (IGL) and/or immunoglobulin kappa (IGK); identifying antibody repertoire in the biological sample(s); comparing and, optionally, contrasting the germ-line polymorphisms to the antibody repertoires to identify the subject or population as responsive to a particular vaccine composition; and administering the vaccine composition to the subject or population of subjects.

As used herein, the terms “prevention,” “vaccination,” or “preventing” can refer to the prophylaxis or to the inhibition of a disease or infection, or to the reduction in the onset of one or more symptoms of a disease or infection. When used with respect to an infectious disease, for example, the terms can refer to a prophylactic administration of a vaccine composition, such as those described herein, which tends to increase the resistance of a subject to infection with a pathogen or, in other words, decreases the likelihood that the subject will become infected with the pathogen or, if infected, will decrease the severity of the infection or will decrease symptoms of illness attributable to the infection.

The term “subject” or “patient”, which can be used interchangeably, can refer to any organism to which aspects of the invention can be administered, e.g., for experimental, diagnostic, prophylactic, and/or therapeutic purposes. Typical subjects to which compounds of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted above or another organism that is alive. The term “living subject” refers to the entire subject or organism and not just a part excised (e.g., a liver or other organ) from the living subject.

In embodiments, a subject can be considered responsive to the vaccine composition if the subject mounts an immune response to the vaccine (i.e., antigen therein).

The vaccines and immunogenic compositions can confer an immune response to a patient after immunization. As used herein, the term “immune response” can refer to a humoral immune response and/or cellular immune response leading to the activation or proliferation of B- and/or T-lymphocytes. In some instances, however, the immune responses can be of low intensity and become detectable only when using at least one substance in accordance with the invention. The term “adjuvant” can refer to an agent used to stimulate the immune system of a living organism, so that one or more functions of the immune system are increased and directed towards the immunogenic agent.

The terms “immunize” or “immunization” or similar terms can refer to conferring the ability to mount a substantial immune response against a target antigen or epitope as it is expressed on a microbe or as the isolated epitope or antigen. These terms do not necessarily require that complete immunity be created, but rather that an immune response be produced that is substantially greater than baseline, e.g., where immunogenic compositions of the invention are not administered or where a conventional (influenza) vaccine is administered. For example, a mammal is considered to be immunized against a target antigen, if the cellular and/or humoral immune response to the target antigen occurs following the application of compositions of the invention or according to methods of the invention.

The term “immunological response” to a composition or vaccine denotes the development of a cellular and/or antibody-mediated immune response in the host animal. Generally, an immunological response includes (but is not restricted to) one or more of the following effects: (a) the production of antibodies; (b) the production of B cells; (c) the production of helper T cells; and/or (d) the production of cytotoxic T cells, that are specifically directed to a given antigen or hapten.

Embodiments herein can further comprise the step of administering a vaccine composition to a subject or a population of subjects. The term “administering” can refer to administering to a subject a pharmaceutical composition of a predetermined dose (e.g., a composition of the invention, such as a vaccine of the first or fourth aspect, a composition of the third aspect, Of the nucleic acid molecule and/or the vector of the sixth aspect). A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. In general, any route of administration may be utilized including

As used herein in reference to a group of individuals, the term “population” can refer to at least 10, 25, 50, 100, 250, 500, 1,000 or more individuals who share a given characteristic (e.g., smokers). As used herein, the term “population” can refer to a plurality of individuals, but does not require that the individuals live in the same locale. Additionally in reference to the methods of the present disclosure, the phrase “administering to a population” does not require that the population receive the immunogenic composition at the same locale or at the same time. That is the individuals of the defined population simply receive the defined immunogenic composition according to the defined immunization schedule.

In embodiments the vaccine can be against a virus. The term “virus”, for example, can refer to an infectious agent that cannot grow or replicate outside the host cell and infects mammals (e.g., humans) or birds. In some embodiments, the infectious agent can cause cancer. Non-limiting examples of such viruses relevant to inventions described herein comprise adenovirus, anthrax, cholera, diphtheria, hepatitis A, hepatitis B, Haemophilus influenza type b, human papillomavirus, season influenza, Japanese encephalitis, measles, meningococcal, mumps, pertussis, pneumococcal, polio, rabies, rotavirus, rubella, shingles, smallpox, tetanus, tuberculosis, typhoid fever, varicella, yellow fever, zika virus.

A subject or population of subject can also be administered a vaccine discovered by methods described herein.

The skilled artisan will recognize that the methods described herein can be utilized generally to inform our understanding of the functional B cell responses in disease processes, thus helping to direct better clinical care, such as the design of more effective therapeutic and prophylactic strategies. For example, the methods described herein can be utilized to treat and/or prevent infectious diseases, along with other diseases such as cancer and autoimmunity.

Kits

As used herein, “kit” can refer to a set of reagents (i.e., components of the kit) for performing the method embodiments of this disclosure. For example, the reagents can include those described in embodiments and examples herein.

The kit can include a box or container that houses the components of the kit. The box or container can be affixed with a label or protocol, such as a label or protocol approved by the Food and Drug Administration. The box or container can contain the components of the present disclosure preferably contained within a plastic, polyethylene, polypropylene, ethylene or propylene container. The container can be a capped tube or bottle.

The kit can also include information material, such as instructions for performing the method embodiments of the disclosure. The informational material can be descriptive, instructional, marketing or other material that relates to the methods described herein and/or the use of the components of the kit.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the vaccine composition, e.g., in a suitable dose, dosage form, or mode of administration (e.g., a dose, dosage form, or mode of administration described herein). The information can be provided in a variety of formats, include printed text, computer readable material, video recording, or audio recording, or an information that provides a link or address to substantive material.

The components in the kit can include other ingredients, such as a solvent or buffer, a stabilizer, or a preservative. The components can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent. The solvent, e.g., sterile water or buffer, can optionally be provided in the kit.

The kit can include one or more containers for the components of the kit, such as the vaccine composition or other components. In some embodiments, the kit contains separate containers, dividers or compartments for the components and informational material. For example, the components can be contained in a bottle, vial, or syringe, and the informational material can be contained in a plastic sleeve or packet. In other embodiments, the separate elements of the kit are contained within a single, undivided container. For example, the components are contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The kit includes a plurality of syringes, tubes, ampules, foil packets, blister packs, or medical devices. The containers of the kits can be air tight, waterproof (e.g., impermeable to changes in moisture or evaporation), and/or light-tight. The kit optionally includes a device suitable for administration of the vaccine composition, e.g., a syringe or other suitable delivery device. The device can be provided pre-loaded with one or both of the agents or can be empty, but suitable for loading.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

The Molecular Basis for Antibody Diversity

Antibodies (Abs) have long been appreciated as key constituents of the adaptive immune response. Their function is to allow selective recognition and mediate immune responses to new foreign antigens. This is accomplished through the somatic generation of vast repertoires of hundreds of millions of unique Ab receptors that can be selected, matured, and ultimately participate in the formation of long-term memory during B-cell development and activation. As a consequence of this diversity, even after nearly a century of research, the complexity of the Ab response within and between individuals is only beginning to be delineated at the molecular and genetic levels.

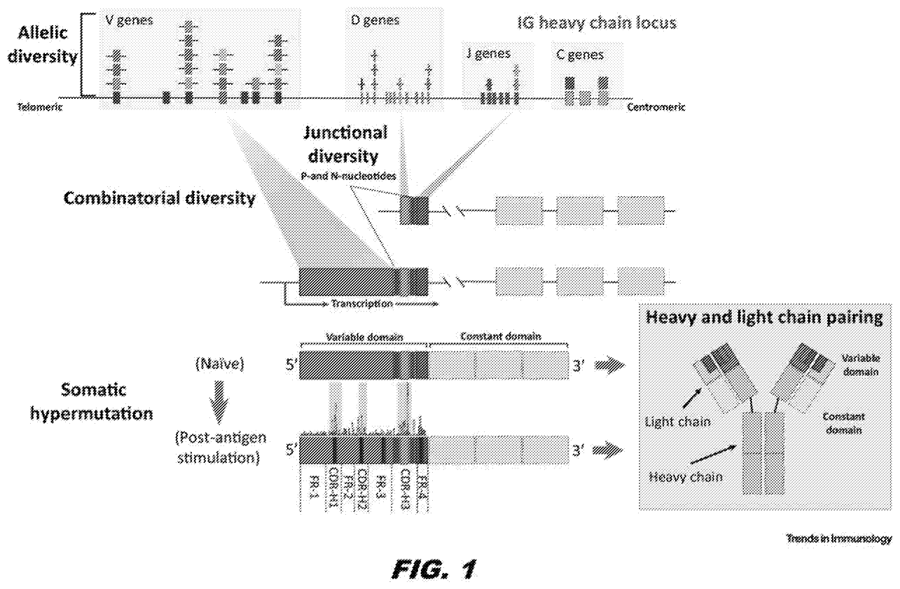

Hundreds of variable (V) and dozens of diversity (D) and joining (J) immunoglobulin (IG) germ-line gene segments across three primary loci in the human genome comprise the necessary building blocks of the expressed Ab heavy- and light-chain repertoires [1]. Whereas the heavy chain is encoded by genes at the IG heavy-chain locus (IGH), the light chain can be encoded by genes at either the IG kappa (IGK) or IG lambda (IGL) chain loci [1]. The naïve Ab repertoire is formed by assembling variants of these building blocks using a specialized V(D)J recombination process that somatically joins various V, D, and J segments (or V and J at IGK and IGL). The introduction and deletion of P and N nucleotides at V(D)J junctions and the pairing of different heavy and light chains dramatically increase diversity (FIG. 1) [2]. Considering these processes alone, a given baseline or primary naïve repertoire can theoretically sample from 1015 different Abs [3]. The extraordinary diversity of the naïve repertoire ensures that it will likely contain a naïve Ab with at least weak initial binding against a vast array of antigens.

TABLE 1
Allelic, Copy number, and Amino Acid Variation for IG Functional
and Open Reading Frame Genes Cataloged in IMGT ^{a}

| | | | | | CDR | CDR- | Genes |
|---|---|---|---|---|---|---|---|
| | | | NS | S | H1 NS | H2 NS | in |
| Family | Genes | Alleles | variants | variants | variants | variants | CNV |
| IGHV1 | 11 | 40 | 19 | 13 | 2 | 3 | 6 |
| IGHV2 | 4 | 23 | 26 | 9 | 3 | 1 | 1 |
| IGHV3 | 27 | 109 | 82 | 57 | 9 | 17 | 12 |
| IGHV4 | 10 | 78 | 92 | 71 | 11 | 8 | 8 |
| IGHV5 | 2 | 9 | 4 | 4 | 0 | 0 | 1 |
| IGHV6 | 1 | 2 | 0 | 1 | 0 | 0 | 0 |
| IGHV7 | 2 | 6 | 4 | 0 | 0 | 0 | 1 |
| Subtotal | 58 | 267 | 227 | 155 | 25 | 29 | 29 |
| IGKV1 | 20 | 35 | 33 | 17 | 4 | 1 | 1 |
| IGKV2 | 11 | 18 | 14 | 4 | 1 | 1 | 0 |
| IGKV3 | 8 | 18 | 24 | 9 | 2 | 1 | 0 |
| IGKV4 | 1 | 1 | NA | NA | NA | NA | 0 |
| IGKV5 | 1 | 1 | NA | NA | NA | NA | 0 |
| IGKV6 | 3 | 5 | 2 | 0 | 0 | 0 | 0 |
| IGKV7 | 0 | 0 | NA | NA | NA | NA | 0 |
| Subtotal | 44 | 78 | 73 | 30 | 7 | 3 | 1 |
| IGLV1 | 7 | 12 | 4 | 2 | 0 | 2 | 1 |
| IGLV2 | 6 | 20 | 13 | 8 | 2 | 3 | 0 |
| IGLV3 | 11 | 18 | 14 | 5 | 3 | 3 | 0 |
| IGLV4 | 3 | 6 | 2 | 1 | 0 | 0 | 0 |
| IGLV5 | 5 | 10 | 3 | 2 | 0 | 0 | 1 |
| IGLV6 | 1 | 2 | 2 | 0 | 0 | 0 | 0 |
| IGLV7 | 2 | 3 | 1 | 0 | 0 | 0 | 0 |
| IGLV8 | 1 | 3 | 1 | 1 | 0 | 0 | 1 |
| IGLV9 | 1 | 3 | 0 | 2 | 0 | 0 | 0 |
| IGLV10 | 1 | 3 | 4 | 1 | 1 | 0 | 0 |
| IGLV11 | 1 | 2 | 1 | 1 | 0 | 0 | 0 |
| Subtotal | 39 | 82 | 45 | 23 | 6 | 5 | 3 |
| Total | 141 | 427 | 345 | 208 | 38 | 40 | 33 |

^{a} Data accessed from IMGT February 2017.
NS, nonsynonymous;
S, synonymous.

Even so, this impressive baseline diversity can be subsequently augmented when a B cell encounters and is stimulated by an antigen to undergo somatic hypermutation (SHM; FIG. 1), resulting in lineages of tens of thousands of clonally derived affinity maturation variants of the initial Ab. Specifically, SHM introduces somatic mutations throughout the variable portion of the Ab, including targeted hotspots residing within the antigen-contacting hypervariable complementarity-determining regions (CDRs). This process ultimately increases the affinity and specificity of the Ab for binding the target epitope, facilitating a highly focused antigen-specific response.

While the prevailing paradigm for investigating B-cell and Ab-mediated responses has placed emphasis on the importance of the unique molecular mechanisms cited earlier in the generation of key functional Abs, there is a growing appreciation for the fact that IG genes are highly variable at the germ-line level, exhibiting extreme allelic polymorphism and gene copy number variation (CNV) between individuals and across populations [4, 5, 6, 7, 8, 9]. Recent studies have begun to highlight that, in addition to diversity introduced during V(D)J recombination, heavy- and light-chain pairing, and SHM, IG germ-line variation (e.g., allelic variation; FIG. 1) plays a vital part in determining the development of the naïve repertoire, with downstream impacts on signatures observed in the memory compartment, and the capacity of an individual to mount an Ab response to specific epitopes [10, 11, 12, 13, 14, 15, 16]

### Example 2

IG Loci Haplotype Diversity in the Human Population

Recent genomic sequencing indicates that IG loci, specifically IGH, may be among the most polymorphic in the human genome [17]. See, for example, Watson, Corey T., et al., Genes and immunity16.1 (2015): 24, which is incorporated by reference herein in its entirety. Across IGH, IGK, and IGL, there are currently >420 alleles cataloged in the ImMunoGeneTics information system database (IMGT) [18, 19, 20, 21] that have been described from germ-line DNA in the human population, with an enrichment of nonsynonymous variants (Table 1). Although the validity of some alleles in IMGT has been called into question [22], the number of polymorphic alleles continues to grow [11, 23, 24], especially as IG gene sequencing is conducted in increasing numbers of non-Caucasian samples [7, 9, 25]. A recent study conducted in 28 indigenous South Africans identified 122 non-IMGT IGHV alleles [9]. In addition to IG allelic variation and single nucleotide polymorphisms (SNPs), CNVs, including large deletions, insertions, and duplications (˜8-75 Kb in length), are also prevalent in IG regions (Table 1). Using IGH as an example, up to 29 of the 58 functional/open reading frame (ORF) IGHV genes may vary in genomic copy number [4, 6, 7, 11, 26, 27, 28]; CNVs of IGH D (diversity) and constant (C) region genes are also known [11, 12, 29]. Until recently, primarily due to technical difficulties associated with the complex genomic architecture of the IG loci, none of the known CNVs in IGHV had been sequenced at nucleotide resolution [7]; many likely remain undescribed at the genomic level. See, for example, Watson, Corey T., et al., The American Journal of Human Genetics 92.4 (2013): 530-546, which is incorporated by reference herein in its entirety.

The high prevalence of IG allelic and locus structural diversity translates into extreme levels of inter-individual haplotype variation [4, 5, 6, 7]. For example, recent comparisons of the two available completed assemblies for the IGHV gene region (˜1 Mb in length) revealed that two human chromosomes can vary by >100 Kb of sequence, with >2,800 SNPs, and CNVs of 10 IGHV functional/ORF genes [7, 17]. In population sequencing experiments, extreme examples of heterozygosity have been noted, with evidence of some individuals carrying more than one allele at every IGHV coding gene [9]. Supporting earlier genetic mapping data [4, 5], more recent analysis of inferred haplotypes from Ab repertoire data surveyed in nine individuals revealed that all 18 haplotypes characterized were unique [6]. Furthermore, at the population level, of the few SNPs and CNVs screened within IGH, allele and genotype frequencies have been shown to vary considerably between ethnic backgrounds [7, 8, 9, 15], with evidence of selection [7]. Despite the evidence for elevated germ-line diversity, genomic resources for IG loci continue to lag behind other regions of the genome [26]. Because of this, the comprehensive and accurate genotyping of IG polymorphisms remains a significant challenge [26, 30], and as a result, the full extent of IG polymorphism and the implications for human health are yet to be uncovered [26]. See, for example, Watson, C. T., and F. Breden, Genes and immunity 13.5 (2012): 363, which is incorporated by reference herein in its entirety. However, it is plausible that population-level diversity in the IG loci, particularly in IGH, will rival that of other complex immune gene families, such as the human leukocyte antigen (HLA) and killer cell IG-like receptor (KIR) genes. These genes are also characterized by extreme haplotype diversity, due to CNV and coding region variation [31, 32]; HLA genes, for example, have thousands of known alleles [31]. In contrast to IG genes, HLA and KIR have been studied more extensively across human populations, and have demonstrated critical roles in disease [31, 32].

### Example 3

Influence of IG Germ-Line Diversity in the Expressed Ab Repertoire and Ab Function

Our limited knowledge of IG population diversity has hindered our ability to comprehensively test for direct connections between IG germ-line polymorphisms, variation in the repertoire generated after recombination, amino acid variation in the Ab produced, and ultimately Ab function. Advances in high-throughput sequencing technology now allow extensive characterization of the expressed Ab repertoire [33, 34, 35], creating opportunities for beginning to investigate the heritability of the Ab response at fine-scale resolution. Applications of these methods, collectively referred to as repertoire sequencing (‘IgSeq’ or ‘RepSeq’), have already led to a wealth of new discoveries in a range of contexts [33, 36]. These include general observations that key features of the Ab repertoire show extensive variability between healthy individuals [10, 11, 13, 14, 37], and a limited overlap of B-cell receptor clones between individuals, even monozygotic (MZ) twins [10, 13, 14]. However, RepSeq studies have also revealed that these inter-individual differences are not necessarily random, but likely have a strong underlying genetic component, providing initial support for the importance of germ-line IG polymorphism in determining the naïve and Ag-stimulated Ab repertoire. For example, several recent studies have revealed that V, D, and J gene usage in the naïve repertoire is much more highly correlated between MZ twins than between unrelated individuals [10, 13, 14], and that IG gene usage patterns are consistent across time points within a given individual [38]. A role for genetic factors can be seen for other repertoire features in twins as well, including the degree of SHM [13], and the distribution of CDR-H3 length and clone convergence [10, 13, 14]. Intriguingly, although existing data suggest that features in the memory compartment are more stochastic, likely reflective of random recruitment and transient proliferation, certain genes and repertoire features exhibit patterns even in memory B cells [10, 13, 14, 39].

Studies of repertoire heritability are consistent with a number of examples for which germ-line IG polymorphisms have been explicitly linked to features in the expressed Ab repertoire [12, 15, 40, 41, 42] (see Figure IA in Box 1 for examples of IG genotype effects on the repertoire). Sasso et al. [40] reported the first direct connection to IG genotype, reporting that CNV of IGHV1-69 was tightly correlated with its relative usage in tonsillar B cells. Our own work has also demonstrated this relationship, but uncovered associations for IGHV1-69 coding and noncoding polymorphism as well as CNV [15]. See, for example, Avnir, Yuval, et al., Scientific reports 6 (2016): 20842, which is incorporated by reference herein in its entirety. Inferred deletions of IGHD genes have also been shown to associate with variation in D-J pairing frequencies, demonstrating that germ-line effects on the repertoire extend beyond V genes [12]. An interesting aspect of IGH CNVs is that, in addition to observed effects of these variants on the genes within the CNV event, they also can impact the usage of genes elsewhere in the locus [12, 15]. For example, we recently observed apparent long-range effects of IGHV1-69 CNV in the naïve and memory repertoire, in that individuals with fewer IGHV1-69 germ-line copies and reduced usage showed consistently higher usage of IGHV genes over 200 Kb away [15]. The mechanisms underlying the observed effects of CNVs in human IG loci remain technically difficult to assess experimentally, but it has been speculated that these large changes in locus architecture (i.e., deletions and insertions) could alter regulatory systems related to V(D)J recombination [12, 15], for example, by modifying the chromatin landscape, cis-regulatory elements and transcription factor binding, and/or the physical locations of the IG V, D, and J genes. All of these factors are known to be key determinants of IG gene accessibility and usage frequencies in mice [43, 44].

### Example 4

Influence of IG Germ-Line Polymorphism on Ab Repertoire Variation and Functional Ab Structural Residues

Although the roles of IG germ-line variants have not been comprehensively studied, there is now convincing evidence that they can influence Ab repertoire variation and function in two main ways (i and ii). In addition, known functional variants exhibit allele frequency variation between human populations (iii):

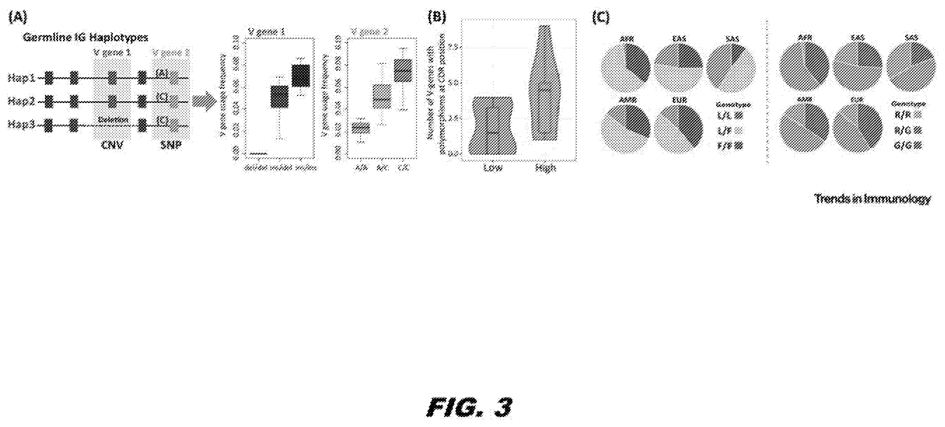

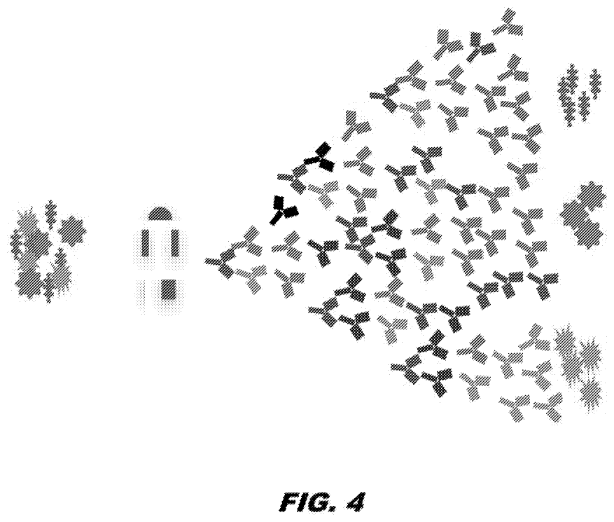

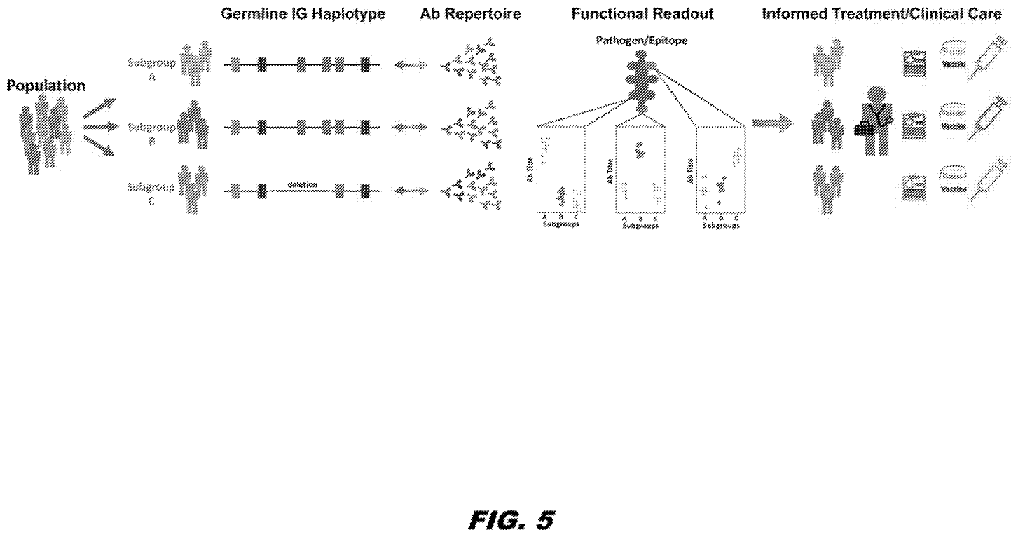

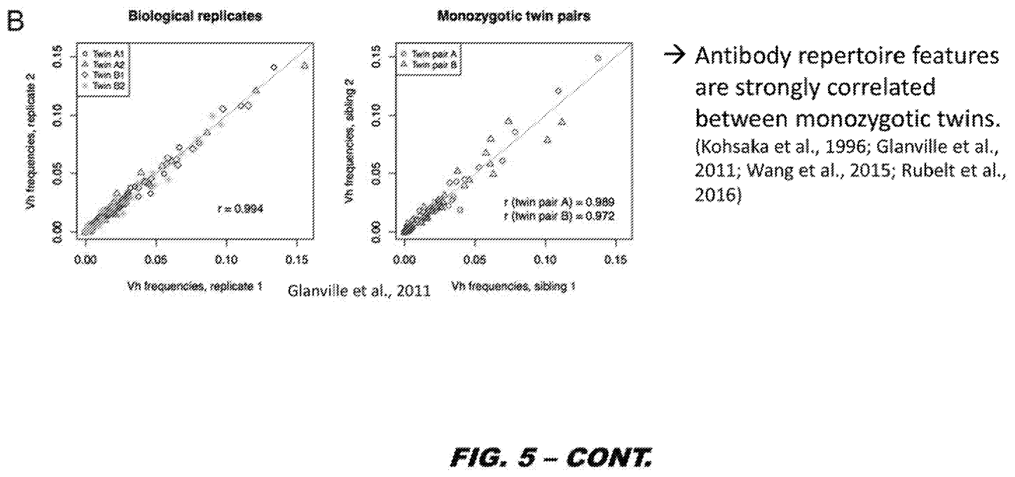

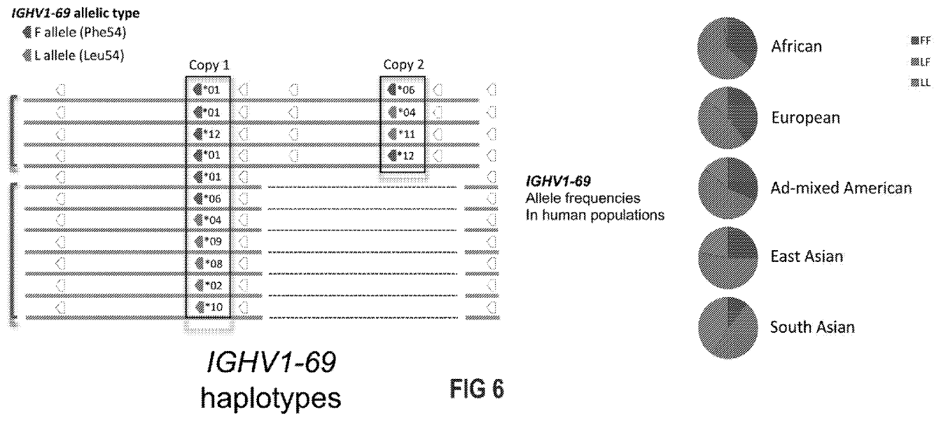

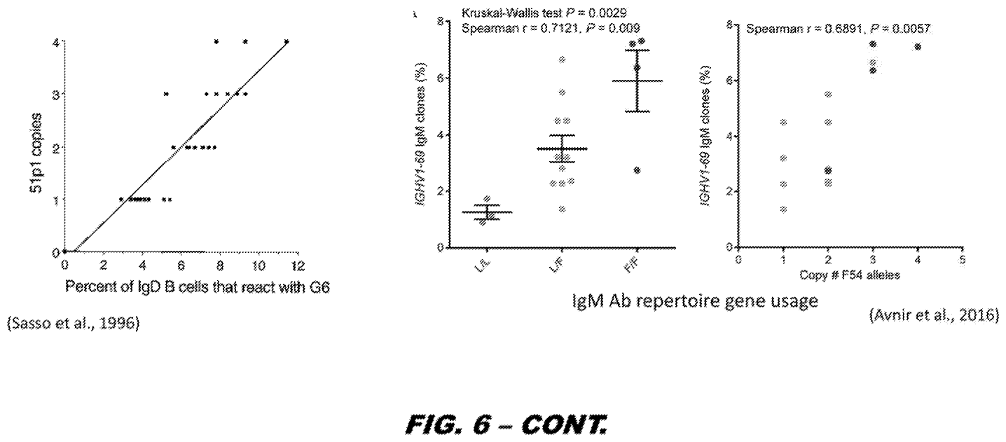

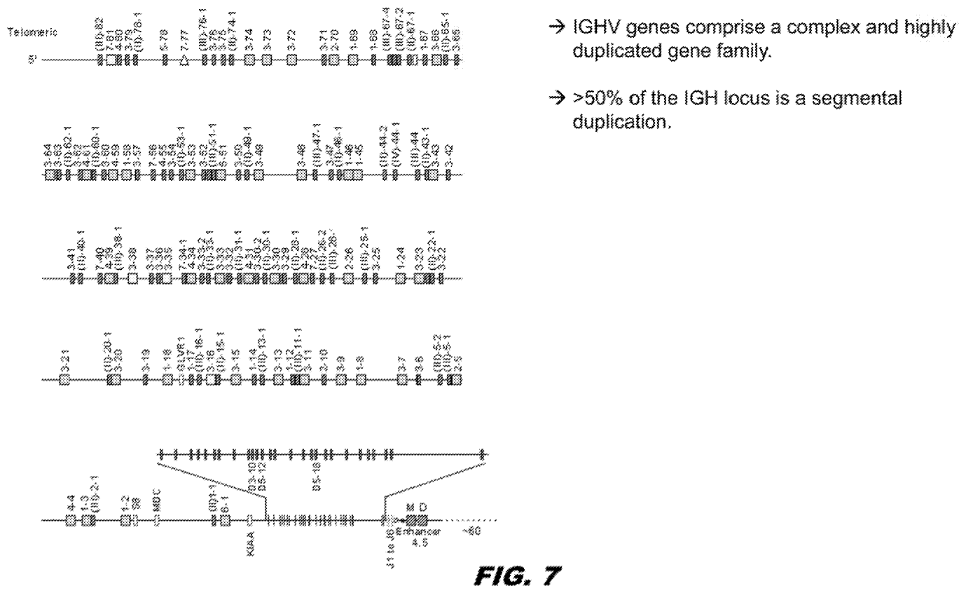

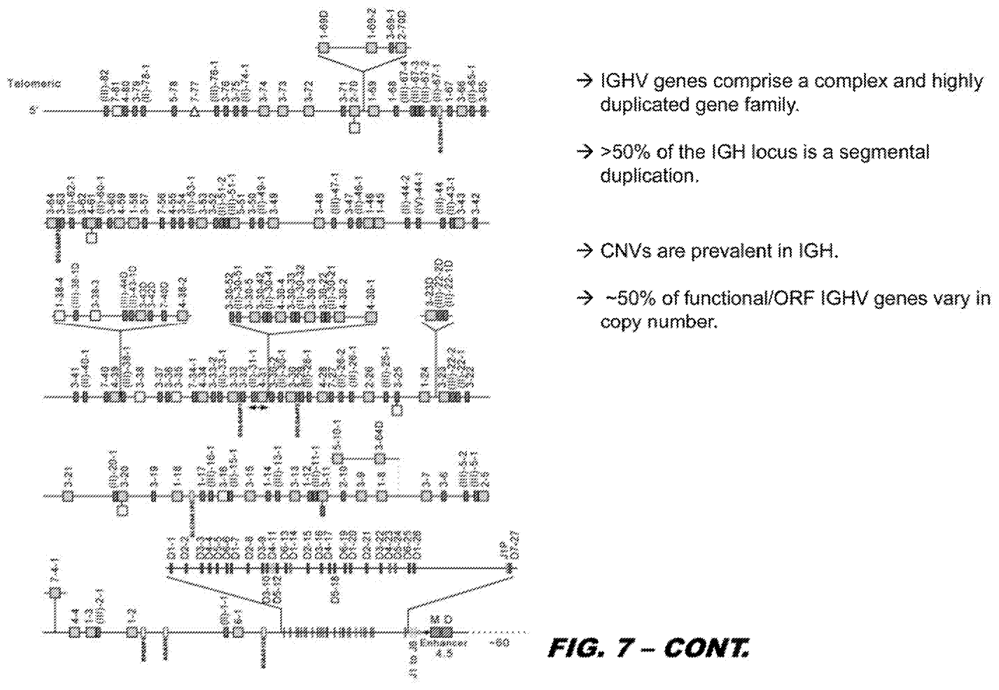

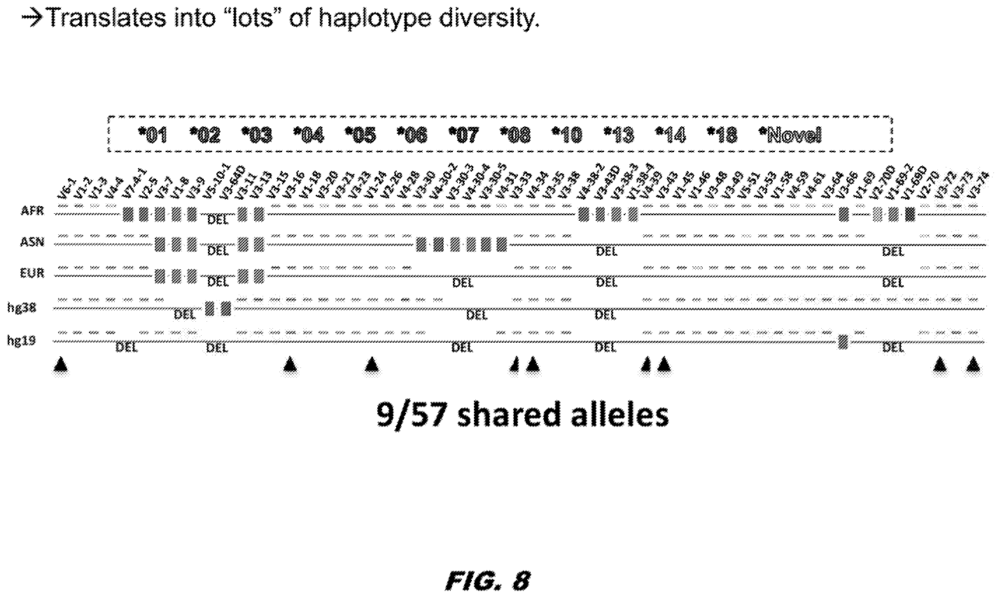

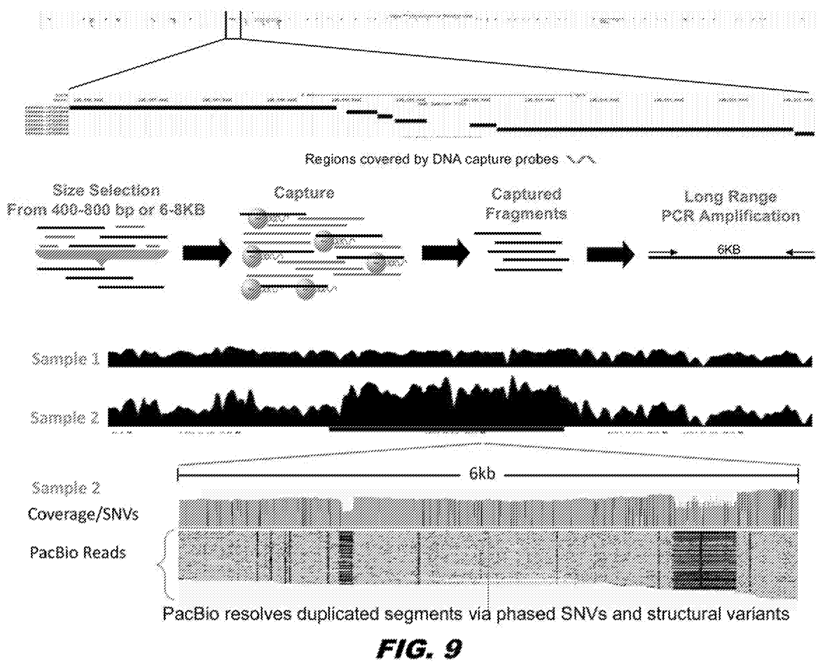

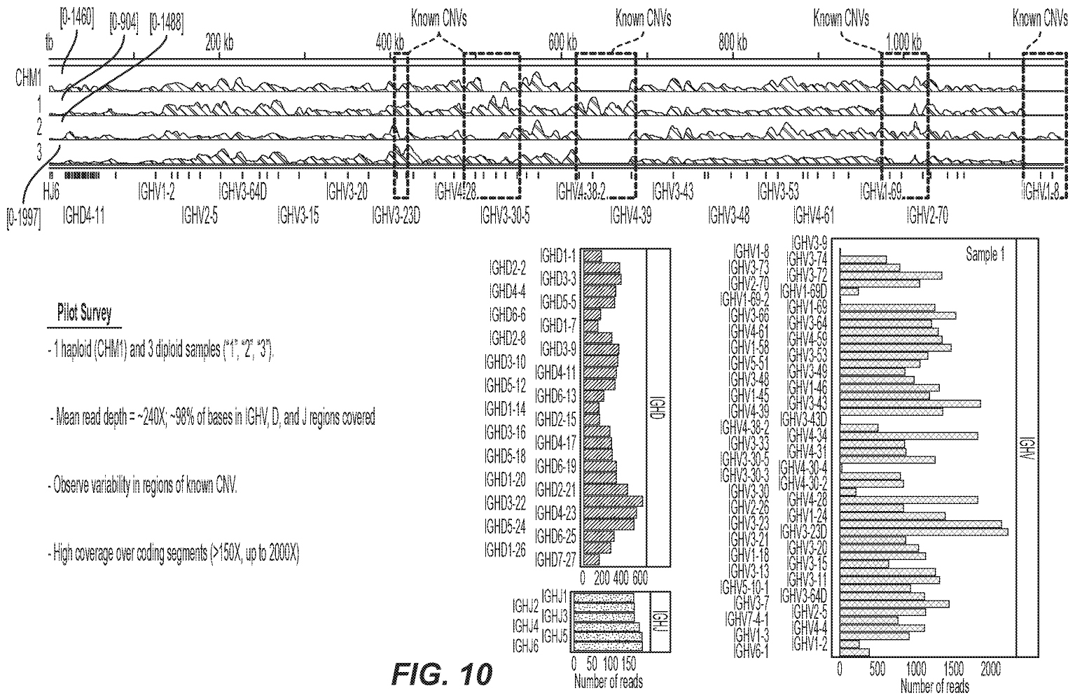

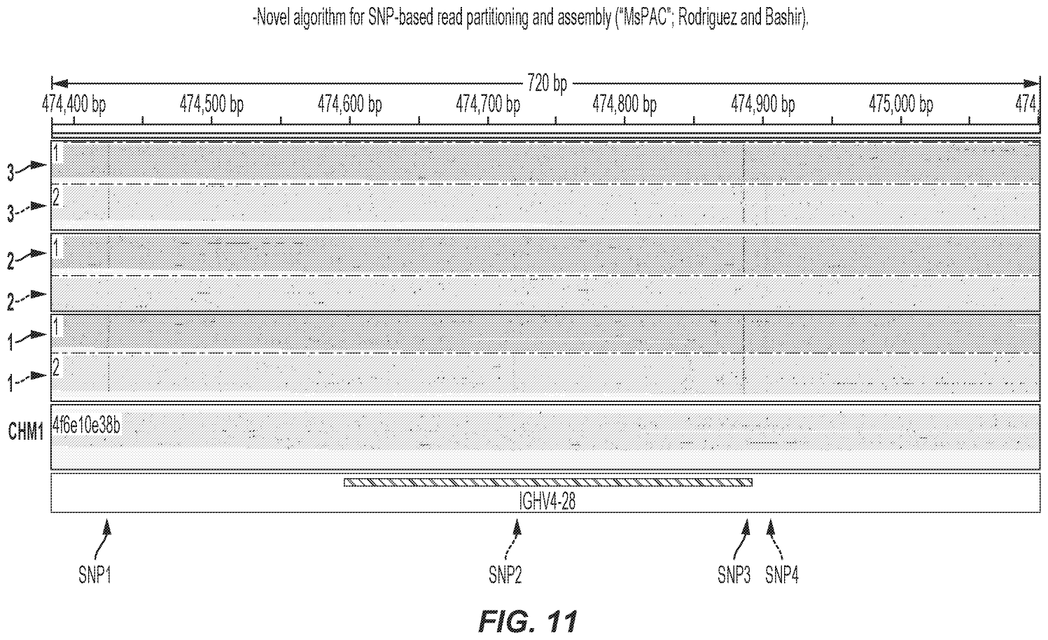

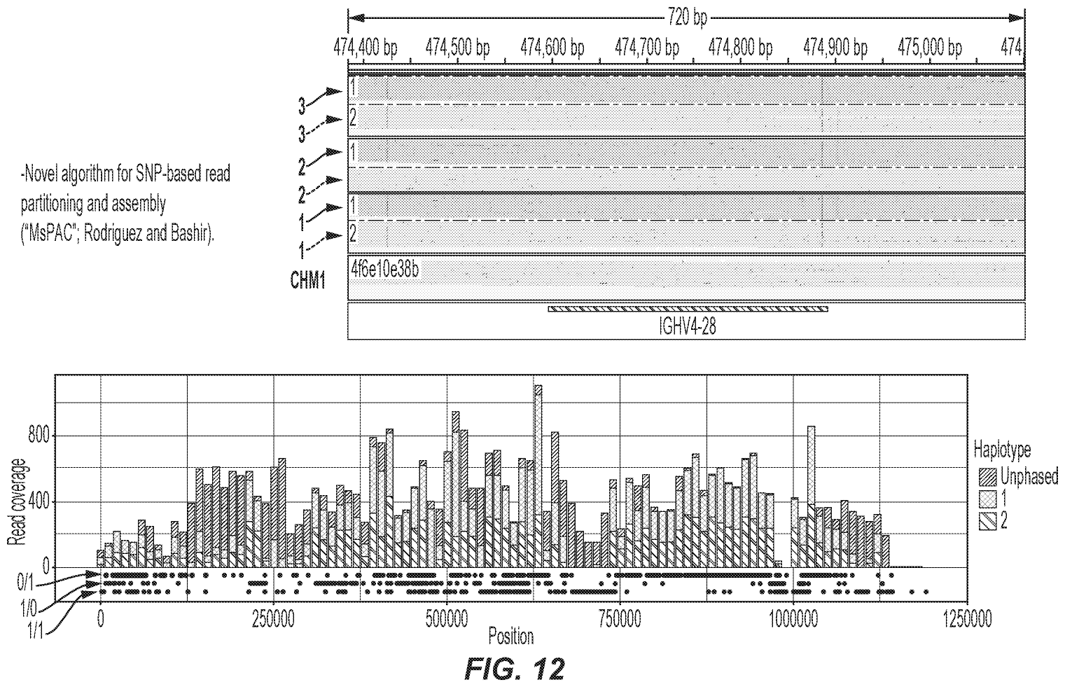

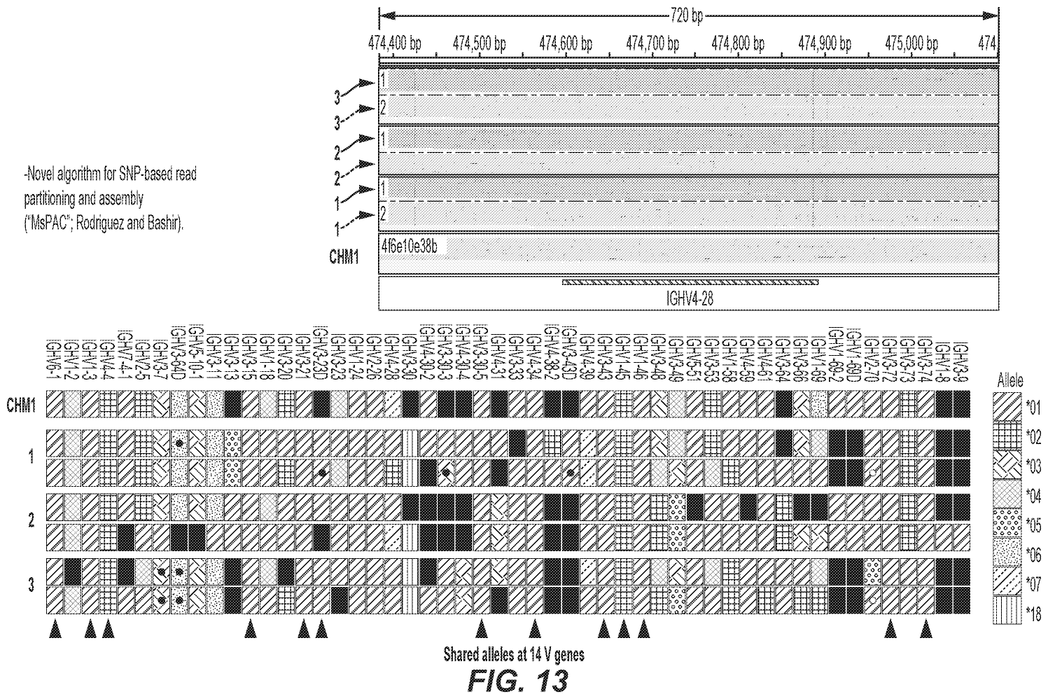

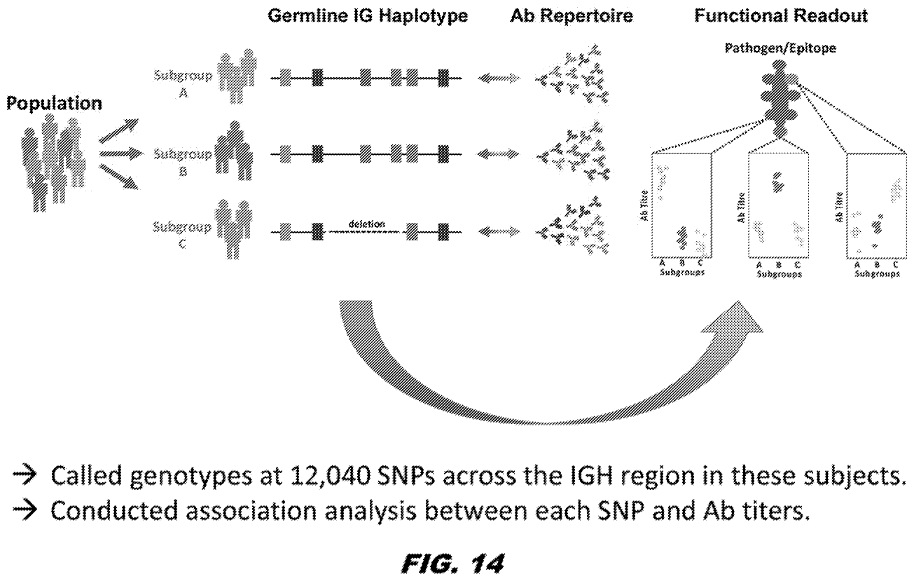

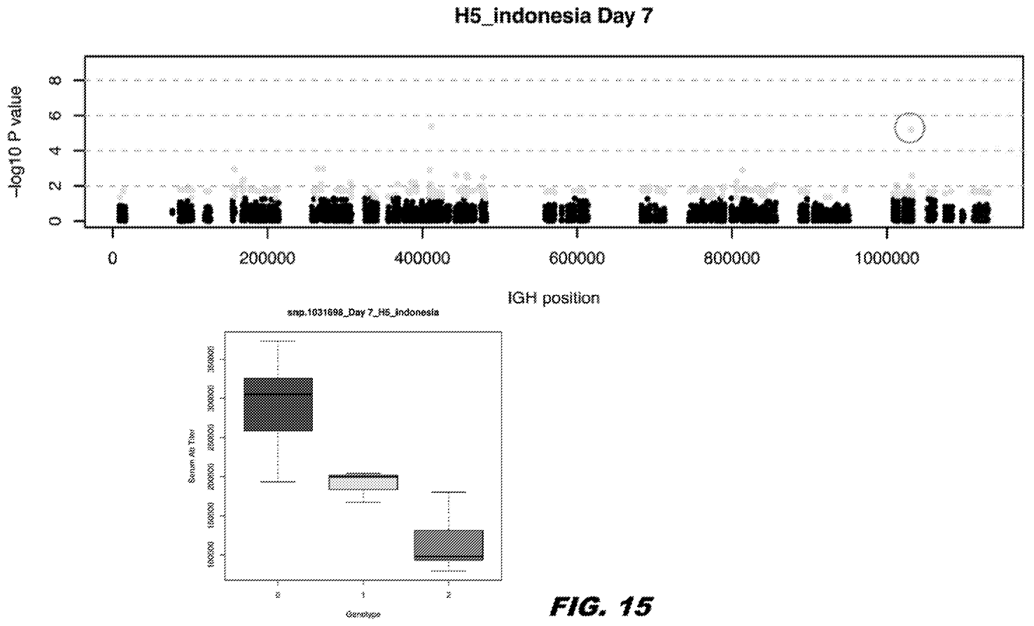

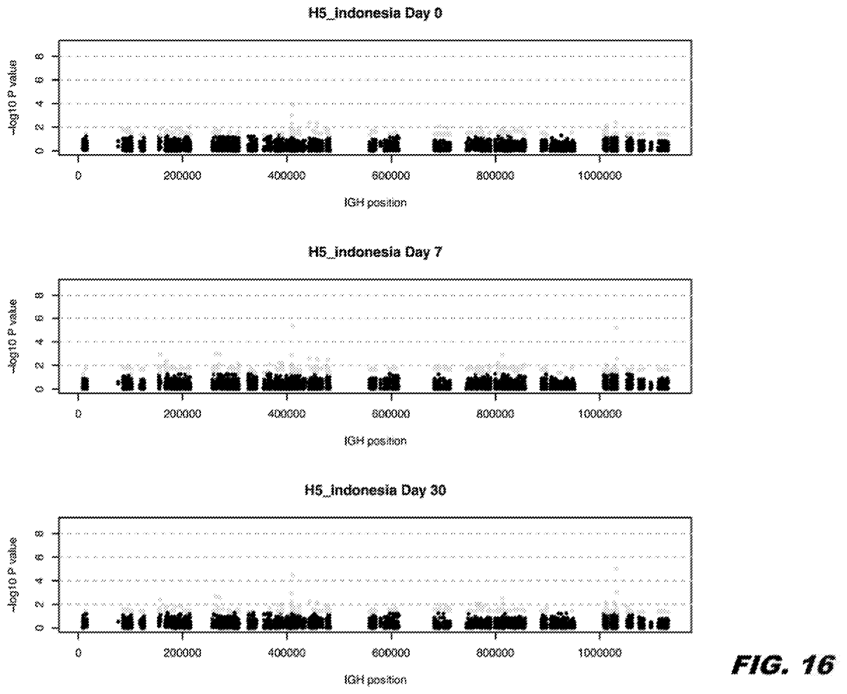

(i) Gene copy number changes and coding/noncoding SNPs in IGHV genes have been shown to correlate with gene usage patterns in the naïve repertoire, the memory repertoire, patterns of SHM, class-switch frequency, and circulating Ab titers (FIG. 3A).

(ii) There are now many examples that provide evidence for functional effects of germ-line variants encoded in CDR-H1 and CDR-H2, many of which are polymorphic and vary between human populations. Based on known IGHV alleles in the IMGT database, residues within CDR-H1/H2 that have a higher probability of making Ag contact are also more likely to be associated with a polymorphic allele (FIG. 3B).

(iii) Several positions in IGHV genes that encode residues critical for antigen binding are polymorphic and exhibit different genotype frequencies between human populations and ethnicities (FIG. 3C).

A role for noncoding polymorphisms is also strongly supported by early work conducted in the human IGK region which directly showed that a variant associated with Haemophilus influenzae infection susceptibility in the recombination signal sequence (RSS) of IGKV2-29 significantly decreased gene rearrangement frequency [42]. RSSs, which are critical for the recruitment of RAG1/2 proteins, have also been demonstrated to impact IGHV gene usage in mice [43, 44]. Moreover, extensive work in the murine IG gene loci has uncovered important roles for other key cis-regulatory sequences and transcription factors as well [45, 46]. Such analyses have not yet been comprehensively conducted in humans, and as a result, our knowledge of the IG regulatory elements involved in the formation of the expressed Ab repertoire is restricted to canonical RSS, promoter, enhancer elements, and class switch regions. However, even for these well-known noncoding regulatory regions, limited data on human population-level variation exist, and thus the broader consequences of polymorphism in these elements on Ab repertoire variability have not been explored.

Although direct links between repertoire variability and human IG CNVs and noncoding polymorphisms remain limited to the few examples discussed above, additional evidence from expressed Ab repertoire studies in unrelated individuals also highlights the ability for these variants to have pervasive impacts on Ab repertoire features, particularly gene usage in the naïve compartment. Most demonstrable is the fact that many of the genes with the most variability in naïve repertoire usage across individuals are also known to be in CNV, including examples of the complete absence of genes in the expressed Ab repertoires of some donors [6, 10, 11, 12]. In addition, allele-specific usage in the naïve Ab repertoires of individuals heterozygous at a given IGHV gene has been demonstrated, also clearly suggesting a role for noncoding variation and CNV [11]. Moreover, although effects of germ-line IG polymorphism may be most evident on a per gene basis, it is worth noting that findings from MZ twins demonstrated that certain CDR-H3 features are highly heritable [13, 14]. This indicates that even strong genetically determined biases on individual V, D, and J gene usage [and thus their nonrandom combination during V(D)J rearrangement] could also be directly linked to variation observed within CDR-H3. This is an important point given that CDR-H3 variation has classically been considered independent of the germ line [13, 14].

In addition to effects of IG polymorphism on gene usage, functional CDR variants can also be directly encoded in the genome. For example, across the ˜267 coding alleles cataloged in IMGT for functional and ORF IGHV genes, 60% of the 382 polymorphisms are nonsynonymous (Table 1), including sites located in CDR-H1 and CDR-H2 with relevance to Ab functional residue diversity (see FIG. 3B). Although the CDR-H3 loop, formed at the V(D)J junction, is the most diverse region of an Ab and is a principal determinant of specificity [47, 48], there is a growing appreciation for the importance of residues outside of CDR-H3 in antigen recognition and binding [15, 49, 50, 51]. For example, recent analyses have shown that the median length of CDR-H2, which is solely encoded by germ-line V gene sequence, is substantially longer than that of CDR-H3, and typically forms the same number of interactions with antigen [52]. Specifically, analyses of antigen-binding region (ABRs; which roughly correspond to CDRs, but differ slightly in their boundaries) have shown that Abs contain a median of six, six, and four contact residues in the heavy-chain CDR-H3, H2, and H1 ABR regions, respectively. In addition, the overall percentage of energetically important Ag-binding residues within each ABR follows the same rank order, with ˜31%, 23%, and 14% for H3, H2, and H1, respectively. Similar trends were noted for light-chain ABRs as well [52]. In addition, considering that many known nonsynonymous sites reside outside of CDRs (Table 1), it is worth highlighting the fact that there are also examples demonstrating indirect effects of framework region variants on Ag binding [53, 54].

### Example 5

The Identification of Shared Ab Immune Response Signatures Across Individuals

A critical question is whether the germ-line effects on the repertoire outlined above can also partially account for inter-individual variation of the Ab-mediated response in disease and clinical phenotypes. The initial observation from RepSeq studies that essentially no Ab clones were shared among individuals, including MZ twins, posed a challenge to comparative Ab repertoire analysis: how could correlates of protection be identified in the Ab repertoire if every individual was responding with different Abs? However, an answer began to emerge with the observation that in multiple settings, including viral and bacterial infection, different individuals have been shown to respond to a given antigen with Abs that share convergent amino acid signatures [13, 49, 54, 55, 56, 57, 58]. These convergent Abs are often encoded by common V genes or sets of V genes, and specific amino acid residues in their CDRs allow them to converge upon a common binding solution against a shared antigen. Critically, in some cases evaluated, convergent signatures include amino acid residues that are directly encoded in the germ line. The occurrence of such convergent Ab responses highlights the ability for tracking common immune responses across individuals, and understanding the role of genetic factors, even when each individual creates unique Abs. Importantly, the implications of this line of thinking could be broad, as IG gene biases have been observed in contexts other than infection, including autoimmunity and cancer [59, 60]. Moreover, IG gene biases may also extend to usage patterns of D and J genes, light-chain genes, and heavy- and light-chain V gene pairing frequencies [56, 61, 62].

### Example 6

Structural Residues Critical for Ag Binding and Involved in Biased Gene Usage are Encoded in the Germ Line and Exhibit Population Variability

There are now many instances for which functional contributions of biased IG genes have been traced back to specific germ-line-encoded residues, including sites that are polymorphic in the human population [15, 16, 50, 53, 54, 55, 63, 64, 65]. These examples illuminate a direct role of the IG germ line in disease-associated Ab responses. In the case of stem-directed broadly neutralizing Abs (BnAbs) against influenza hemagglutinin (HA), the most prevalent Abs use the heavy-chain gene IGHV1-69 [66, 67, 68, 69, 70]. These IGHV1-69 BnAbs recognize an overlapping epitope of group 1 influenza A viruses and only amino acids from IGHV make contact with HA. Importantly, of the 14 known alleles at IGHV1-69, only those encoding a critical phenylalanine at position 54 (F54) within CDR-H2 have a major role in shaping the BnAbs response [16, 15, 55, 71]. Although IGHV1-69 F54-encoding alleles are dominant, there is a growing list of additional HA-directed BnAbs that also show IG germ-line biases [51, 56, 72, 73, 74], including those also known to be polymorphic with respect to coding variants and CNVs.

Interestingly, there are additional instances of biased IGHV1-69 allele usage in other disease contexts, with both overlapping and contrasting patterns to that observed for influenza. For example, F54 alleles are predominantly observed in IGHV1-69-expressing B cells associated with chronic lymphoid leukemia (CLL), whereas alleles encoding a leucine (L54) at this position are primarily used by non-neutralizing anti-gp41 Abs in HIV-1 [63, 64]. Moreover, it has been shown that IGHV1-69 F54 alleles, in comparison with L54 alleles, have lower usage in the memory B-cell pool [10, 15]. This observation may be similar to trends noted for IGHV4-34, which is also significantly underrepresented in the memory compartment of healthy individuals [10], and presumes to reflect a selective pressure against autoreactive Abs [75, 76].

Other polymorphic positions in the framework regions of IGHV1-69, in conjunction with CDR-H2 54, have also recently been shown to influence Ab binding of Middle East respiratory syndrome coronavirus (MERS-CoV) [53] and the Staphylococcus aureus NEAr iron transporter 2 (NEAT2) domain [54]. In the example of NEAT2, neutralizing Abs encoded by IGHV1-69 alleles carrying an arginine (R) at position 50 in place of glycine (G) showed significantly reduced NEAT2 binding [54]. Interestingly, based on publicly available data, the frequencies of critical alleles within polymorphic positions of IGHV1-69 vary across populations (see FIG. 3C).

### Example 7

A Strategy for Defining Relationships between IG Polymorphisms, Expressed Ab Signatures, and Functional Outcomes

Considering the aforementioned evidence, we argue that the antigen-specific Ab repertoire is likely influenced by the host genotype. Although the genetic bases for repertoire and germ-line gene biases have not been comprehensively investigated, several recent studies provide a strategy for systematically integrating data on IG polymorphism and Ab responses at the population and molecular levels to provide unique insight into Ab signatures associated with disease.

We have begun to explore this idea in detail at the IGHV1-69 locus in the context of influenza vaccination [15]. Providing strong proof-of-concept, by initially focusing on observed IGHV1-69 allelic usage bias against a critical broadly neutralizing epitope, we genotyped the IGHV1-69 F54/L54 allele and copy number frequencies in a cohort of 85 H5N1 vaccines, including 18 individuals with accompanying Ab repertoire data [15]. Drawing directly on aspects of repertoire heritability reviewed above, we found robust connections between these polymorphisms and repertoire gene usage in both the unmutated IgM (naïve) and IgG memory repertoires, with IGHV1-69 germ-line gene usage increasing with the number of copies of F54 alleles. In addition to usage frequencies, IGHV1-69 genotype also associated with IGHV1-69 B-cell expansion, SHM, and Ig class switching. It is important to note that these genotype effects extended to levels of circulating anti-HA stem BnAbs postvaccination, with individuals carrying only germ-line-encoded CDR-H2 L54 alleles having lower IGHV1-69 BnAbs. Furthermore, with direct repertoire sequencing, we were able to specifically demonstrate that only carriers of the IGHV1-69 F54 alleles expressed convergent anti-BnAb signatures. These results are bolstered by similar observations recently made by two other groups that also carried out IGHV1-69 F54/L54 allele genotyping in their cohorts [16, 55]. Altogether, these data demonstrate that genetically determined baseline differences in the Ab repertoire can set the stage for disease-related responses.

In one embodiment, the frequency of IGHV1-69 F54 alleles and CNV varies considerably across populations [7, 15]. Specifically, the number of individuals that would lack the capacity to generate effective IGHV1-69 BnAbs was much higher in some populations. However, we and others have shown that individuals lacking IGHV1-69 F54 alleles likely utilize other germ-line genes in place of IGHV1-69 [51, 55]. This finding in particular both highlights the complexity of the Ab response and demonstrates that the integration of genotyping information can help provide a more nuanced interpretation of the signatures discovered in the expressed repertoire. Moreover, it suggests that efforts should be made to study these complex responses in larger and more diverse cohorts, including individuals from presently understudied populations.

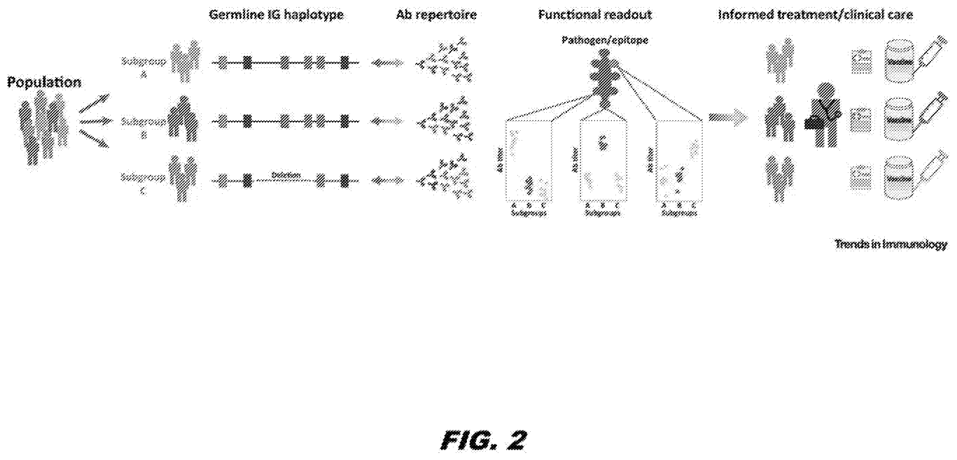

Building on findings in these studies [15, 16, 55], a framework for integrating genotypic information into future studies of the Ab response in wellness and disease is provided (FIG. 2). The general strategy is as follows: (i) identify IG gene biases observed in a disease-related or epitope-specific response; (ii) characterize this response at the population level by performing comprehensive genotyping of coding, noncoding, and gene copy number variants at and around the locus of interest (and others if there is rationale); (iii) perform repertoire sequencing and analysis of the response in all relevant B-cell subsets to identify all Ab convergence groups with allele bias; and (iv) evaluate genotype-phenotype linkages of the functional Ab response and specific Ab convergence groups.

We see a growing body of evidence to support the link between IG polymorphism and phenotype that may have important clinical applications (see Outstanding Questions). The most obvious of these correlations include effects of CNV and SNPs in non-translated and translated IG gene regions on expressed repertoire variability in naïve and memory B cell subsets. Some of these polymorphisms could more broadly impact variation in protective Ab responses [77] and quality of the memory B-cell pool. We anticipate that IG polymorphism will contribute to differences in expression of common (public) and unique (private) antibody signatures that are associated with protective responses in disease and in response to vaccination. Cataloging these signatures for biased gene use, V(D)J associations, SHMs, and heavy-light chain pairing in the context of IG germ-line variation will provide us with information to advance our understanding of the immunogenetic potential of an individual's baseline naïve repertoire (FIG. 2), particularly when more complete data sets of biased Ab signatures to specific epitopes become available. Based on existing genetic data, similar IG haplotypes will associate with overlapping signatures in baseline repertoire profiles, even if not to the degree of repertoire similarity observed in MZ twins. This IG polymorphism, as we and others have begun to show, may further influence the evolution of antigen-experienced B cells and plasma cells, where other genetic polymorphisms in the IG loci and environmental exposures come into play in continuing to shape affinity, epitope specificity, and fate. In addition, class-switched memory B-cell compartments will vary over time [37], and could be quantitated in the type and size of clonotypes with both public and private signatures against immunodominant epitopes.

Together, this knowledge should pave the way to using molecular and genetic signatures for mapping an individual's exposure history, current wellness state, and immune potential against future antigenic threats. For example, characterization of genotypes that specifically lead to common BnAb signatures in the repertoire should be useful for tailoring vaccines to responsive genotypes with the goal of achieving 100% ‘universal vaccine’ responsiveness at the population level (FIG. 2). In addition, such information could lead to advances in the use of anti-idiotypic antibody and chimeric antigen receptor T-cell therapies that are directed against germ-line gene expressing B-cell clonotypes that are directly involved in autoimmune disease and hematologic malignancies [78, 79]. We face tall hurdles to moving this paradigm forward, the greatest being the completion of a comprehensive catalogue of human IG haplotype variation [26]. However, with ever expanding advances in immunologic and genomic technologies, we believe that such integrative approaches are within our reach, and have the ability to transform our understanding of Ab-mediated immune responses in the clinical and research arenas.

### Example 8

Outstanding Questions

How large of an effect does IG polymorphism have on the development of the baseline naïve repertoire, and what types of genetic variation (CNV, coding variants, regulatory variants) matter most?

Do effects of IG genetic variants on the Ab repertoire correspond to known biases in disease and/or clinically relevant Ab responses?

What can population-level data on genetic and expressed Ab repertoire signatures tell us about an individual's exposure history, current wellness state, and immune potential against future antigenic threats?

Can we leverage integrated population-level data sets to inform clinical care, and more effective vaccine and therapeutic strategies?

### REFERENCES CITED IN EXAMPLES 1-8

1. Lefranc, M.-P. and Lefranc, G. (2001) The Immunoglobulin Facts—book, Academic Press
2. Tonegawa, S. (1983) Somatic generation of antibody diversity. Nature 302, 575-581
3. Schroeder, H. W. (2006) Similarity and divergence in the development and expression of the mouse and human antibody repertoires. Dev. Comp. Immunol. 30, 119-135
4. Chimge, N.-O. et al. (2005) Determination of gene organization in the human IGHV region on single chromosomes. Genes Immun. 6, 186-193
5. Li, H. et al. (2002) Genetic diversity of the human immunoglobulin heavy chain VH region. Immunol. Rev. 190, 53-68
6. Kidd, M. J. et al. (2012) The inference of phased haplotypes for the immunoglobulin H chain V region gene loci by analysis of VDJ gene rearrangements. J. Immunol. 188, 1333-1340
7. Watson, C. T. et al. (2013) Complete haplotype sequence of the human immunoglobulin heavy-chain variable, diversity, and joining genes and characterization of allelic and copy-number variation. Am. J. Hum. Genet. 92, 530-546
8. Sasso, E. H. et al. (1995) Ethnic differences in polymorphism of an immunoglobulin VH3 gene. J. Clin. Invest. 96, 1591-1600
9. Scheepers, C. et al. (2015) Ability to develop broadly neutralizing HIV-1 antibodies is not restricted by the germline IG gene repertoire. J. Immunol. 194, 4371-4378
10. Glanville, J. et al. (2011) Naïve antibody gene segment frequencies are heritable and unaltere by chronic lymphocyte ablation. Proc. Natl. Acad. Sci. U.S.A. 108, 20066-20071
11. Boyd, S. D. et al. (2010) Individual variation in the germline Ig gene repertoire inferred from variable region gene rearrangements. J. Immunol. 184, 6986-6992
12. Kidd, M. J. et al. (2015) DJ pairing during VDJ recombination shows positional biases that vary among individuals with differing IGHD locus immunogenotypes. J. Immunol. 196, 1158-1164
13. Wang, C. et al. (2015) B-cell repertoire responses to varicella-zoster vaccination in human identical twins. Proc. Natl. Acad. Sci. U.S.A. 112, 500-505
14. Rubelt, F. et al. (2016) Individual heritable differences result in unique lymphocyte receptor repertoires of naïve and antigen experienced cells. Nat. Commun. 6, 1-12
15. Avnir, Y. et al. (2016) IGHV1-69 polymorphism modulates anti-influenza antibody repertoires, correlates with IGHV utilization shifts and varies by ethnicity. Sci. Rep. 6, 20842
16. Wheatley, A. K. et al. (2015) H5N1 vaccine-elicited memory B cells are genetically constrained by the IGHV locus in the recognition of a neutralizing epitope in the hemagglutinin stem. J. Immunol. 195, 602-610
17. Watson, C. T. et al. (2014) Sequencing of the human IG light chain loci from a hydatidiform mole BAC library reveals locus-specific signatures of genetic diversity. Genes Immun. 16, 24-
18. Pallarès, N. et al. (1999) The human immunoglobulin heavy variable genes. Exp. Clin. Immunogenet. 16, 36-60
19. Lefranc, M.-P. et al. (2014) IMGT1, the international Immunogenetics information system 1 25 years on. Nucleic Acids Res. 43, D413-D422
20. Pallarés, N. et al. (1998) The human immune globulin lambda variable (IGLV) genes and joining (IGLJ) segments. Exp. Clin. Immunogenet. 15, 8-18
21. Barbié, V. and Lefranc, M. P. (1998)The human immunoglobulin kappa variable (IGKV) genes and joining (IGKJ) segments. Exp. Clin. Immunogenet. 15, 171-183
22. Wang, Y. et al. (2008) Many human immunoglobulin heavy-chain IGHV gene polymorphisms have been reported in error. Immunol. Cell Biol. 86, 111-115
23. Gadala-Maria, D. et al. (2015) Automated analysis of high-throughput B-cell sequencing data reveals a high frequency of new immunoglobulin V gene segment alleles. Proc. Natl. Acad. Sci. U.S.A. 112, E862-E870
24. Corcoran, M. M. et al. (2016) Production of individualized V gene databases reveals high levels of immunoglobulin genetic diversity. Nat. Commun. 7, 13642
25. Wang, Y. et al. (2011) Genomic screening by 454 pyrosequencing identifies a new human IGHV gene and sixteen other new IGHV allelic variants. Immunogenetics 63, 259-265
26. Watson, C. T. and Breden, F. (2012)The immunoglobulin heavy chain locus:genetic variation, missing data, and implications for human disease. Genes Immun. 13, 363-373
27. Milner, E. C. et al. (1995) Polymorphism and utilization of human VH genes. Ann. N.Y. Acad. Sci. 764, 50-61
28. Shin, E. K. et al. (1993) Polymorphism of the human immunoglobulin variable region segment V1-4.1. Immunogenetics 38, 304-306
29. Bottaro, A. et al. (1991) Pulsed-field electrophoresis screening for immunoglobulin heavy-chain constant-region (IGHC) multigene deletions and duplications. Am. J. Hum. Genet. 48, 745-756
30. Luo, S. et al. (2016) Estimating copy number and allelic variation at the immunoglobulin heavy chain locus using short reads. PLoS Comput. Biol. 12, 1-21
31. Trowsdale, J. and Knight, J. C. (2013) Major histocompatibility complex genomics and human disease. Annu. Rev. Genomics Hum. Genet. 14, 301-323
32. Parham, P. and Moffett, A. (2013) Variable NK cell receptors and their WIC class I ligands in immunity, reproduction and human evolution. Nat. Rev. Immunol. 13, 133-144
33. Georgiou, G. et al. (2014) The promise and challenge of high-throughput sequencing of the antibody repertoire. Nat. Biotech-nol. 32, 158-168
34. Boyd, S. D. and Joshi, S. A. (2014)High-throughput DNA sequencing analysis of antibody repertoires. Microbiol. Spectr. 2, 1-13
35. Yaari, G. and Kleinstein, S. H. (2015) Practical guidelines for B-cell receptor repertoire sequencing analysis. Genome Med. 7, 121
36. Jackson, K. J. L. et al. (2013) The shape of the lymphocyte receptor repertoire: lessons from the B cell receptor. Front. Immunol. 4, 1-12
37. Galson, J. D. et al. (2015) In depth assessment of within-individual and inter-individual variation in the B cell receptor repertoire. Front. Immunol. 6, 1-13
38. Laserson, U. et al. (2014) High-resolution antibody dynamics of vaccine-induced immune responses. Proc. Natl. Acad. Sci. U.S.A. 111, 4928-4933
39. Vollmers, C. et al. (2013) Genetic measurement of memory B-cell recall using antibody repertoire sequencing. Proc. Natl. Acad. Sci. U.S.A. 110, 13463-13468
40. Sasso, E. H. et al. (1996) Expression of the immunoglobulin VH gene 51p1 is proportional to its germline gene copy number. J. Clin. Invest. 97, 2074-2080
41. Sharon, E. et al. (2016) Genetic variation in MHC proteins is associated with T cell receptor expression biases. Nat. Genet. 48, 995-1002
42. Feeney, A. J. et al. (1996) A defective V kappa A2 allele in Navajos which may play a role in increased susceptibility to Haemophilus influenzae type b disease. J. Clin. Invest. 97, 2277-2282
43. Feeney, A. J. (2009)Genetic and epigenetic control of V gene rearrangement frequency. Adv. Exp. Med. Biol. 650, 73-81
44. Choi, N. M. et al. (2013) Deep sequencing of the murine IgH repertoire reveals complex regulation of nonrandom V gene rearrangement frequencies. J. Immunol. 191, 2393-2402
45. Volpi, S. A. et al. (2012) Germline deletion of Igh30 regulatory region element shs5,6,7(hs5-7) affects B cell-specific regulation, rearrangement, and insulation of the Igh locus. J. Immunol. 188, 2556-2566
46. Verma-Gaur, J. et al. (2012) Non coding transcription within the Igh distal VH region at PAIR elements affects the 3D structure of the Igh locus in pro-B cells. Proc. Natl. Acad. Sci. U.S.A. 109, 17004-17009
47. Xu, J. L. and Davis, M. M. (2000) Diversity in the CDR3 region of V H is sufficient for most antibody specificities. Immunity 13, 37-45
48. Mahon, C. M. et al. (2013) Comprehensive interrogation of a minimalist synthetic CDR-H3library and its ability to generate antibodies with therapeutic potential. J. Mol. Biol. 425, 1712-1730
49. Thomson, C. A. et al. (2008) Germ line V-genes sculpt the binding site of a family of antibodies neutralizing human cytomegalovirus. EMBO J. 27, 2592-2602
50. Bryson, S. et al. (2016) Structures of preferred human Ig V genes-based protective antibodies identify how conserved residues contact diverse antigens and assign source of specificity to CDR3 loop variation. J. Immunol. 196, 4723-4730
51. Fu, Y. et al. (2016) A broadly neutralizing anti-influenza antibody reveals on going capacity of haemagglutinin-specific memory B cells to evolve. Nat. Commun. 7, 12780
52. Kunik, V. and Ofran, Y. (2013) The indistinguishability of epitopes from protein surface is explained by the distinct binding preferences of each of the six antigen-binding loops. Protein Eng. Des. Sel. 26, 599-609
53. Ying, T. et al. (2015) Junctional and allele-specific residues are critical for MERS-CoV neutralization by an exceptional lypotent germline-like antibody. Nat. Commun. 6, 8223
54. Yeung, Y. A. et al. (2016) Germline-encoded neutralization of a Staphylococcus aureus virulence factor by the human antibody repertoire. Nat. Commun. 7, 13376
55. Pappas, L. et al. (2014) Rapid development of broadly influenza neutralizing antibodies through redundant mutations. Nature 516, 418-422
56. Joyce, M. G. et al. (2016) Vaccine-induced antibodies that neutralize group land group2 influenza A viruses. Cell 166, 609-623
57. Parameswaran, P. et al. (2013) Article convergent antibody signatures in human dengue. Cell Host Microbe 13, 691-700
58. Strauli, N. B. and Hernandez, R. D. (2016)Statistical inference of a convergent antibody repertoire response to influenza vaccine. Genome Med. 8, 60
59. Johansen, J. N. et al. (2015) Intrathecal BCR transcriptome in multiple sclerosis versus other neuroinflammation: equally diverse and compartmentalized, but more mutated, biased and over-lapping with the proteome. Clin. Immunol. 160, 211-225
60. Bomben, R. et al. (2010) Expression of mutated IGHV3-23 genes in chronic lymphocytic leukemia identifies a disease subset with peculiar clinical and biological features. Clin. Cancer Res. 16, 620-628
61. Forconi, F. et al. (2013) The IGHV1-69/IGHJ3 recombinations of unmutated CLL are distinct from those of normal B cells. Blood 119, 2106-2109
62. Zhu, D. et al. (2013) Biased immunoglobulin light chain use in the Chlamydophila psittaci negative ocularadnexal marginal zone lymphomas. Am. J. Hematol 88, 379-384
63. Hwang, K. K. et al. (2014) IGHV1-69 B cell chronic lymphocytic leukemia antibodies cross-react with HIV-1 and hepatitis C virus antigens as well as intestinal commensal bacteria. PLoS One 9, e90725
64. Williams, W. B. et al. (2015) HIV-1 vaccines. Diversion of HIV-1 vaccine-induced immunity by gp41-microbiota cross-reactive antibodies. Science 349, aab1253
65. Liu, L. and Lucas, A. H. (2003) IGHV3-23*01 and it sallele V3-23*03 differ in their capacity to form the canonical human antibody combining site specific for the capsular polysaccharide of Haemophilus influenzae type b. Immunogenetics 55, 336-338
66. Throsby, M. et al. (2008) Hetero subtypic neutralizing monoclonal antibodies cross-protective against H5N1and H1N1 recovered from human IgM+ memory B cells. PLoS One 3, e3942
67. Wrammert, J. et al. (2011) Broadly cross-reactive antibodies dominate the human B cell response against 2009 pandemic H1N1 influenza virus infection. J. Exp. Med. 208, 181-193
68. Ekiert, D. C. et al. (2009) Antibody recognition of a highly conserved influenza virus epitope. Science 324, 246-251
69. Kashyap, A. K. et al. (2008) Combinatorial antibody libraries from survivors of the Turkish H5N1 avian influenza outbreak reveal virus neutralization strategies. Proc. Natl. Acad. Sci. U.S.A. 105, 5986-5991
70. Corti, D. et al. (2011) A neutralizing antibody selected from plasma cells that binds to group 1 and group 2 influenza A hemagglutinins. Science 333, 850-856
71. Lingwood, D. et al. (2012) Structural and genetic basis for development of broadly neutralizing influenza antibodies. Nature 489, 566-570
72. Nakamura, G. et al. (2013) An in vivo human plasmablast enrichment technique allows rapid identification of therapeutic influenza A antibodies. Cell Host Microbe 14, 93-103
73. Kallewaard, N. L. et al. (2016) Structure and function analysis of an antibody recognizing all influenza A subtypes. Cell 166, 596-608
74. Wu, Y. et al. (2015) A potent broad-spectrum protective human monoclonal antibody cross linking two haemagglutinin monomers of influenza A virus. Nat. Commun. 6, 7708
75. Pugh-Bernard, A. E. (2001) Regulation of inherently autoreactive VH4-34 B cells in the maintenance of human B cell tolerance. J. Clin. Invest. 108, 1061-1070
76. Cappione, A. J. et al. (2004) Lupus IgGVH 4.34 antibodies bind to a 220-kDa glycoform of CD45/B22 on the surface of human B lymphocytes. J. Immunol. 172, 4298-4307
77. Lee, J. et al. (2016) Molecular-level analysis of the serum antibody repertoire in young adults before and after seasonal influenza vaccination. Nat. Med. 22, 1456-1464
78. Fesnak, A. D. et al. (2016) Engineered T cells: the promise and challenges of cancer immunotherapy. Nat. Rev. Cancer 16, 566-581
79. Chang, D. K. et al. (2016) Humanized mouse G6 anti-idiotypic monoclonal antibody has therapeutic potential against IGHV1-69 germline gene-based B-CLL. MAbs 8, 787-798
80. Auton, A. et al. (2015) A global reference for human genetic variation. Nature 526, 68-74

### Example 9

Leveraging genomic variants in the immunoglobulin gene regions to inform functional antibody responses and associated clinical phenotypes.

Because the world of pathogens is diverse, it is important that antibodies also be diverse. In fact, our immune system can theoretically produce about ˜100,000,000,000 antibodies with different specificities.

We know that from a structural standpoint, the IGHV is one of the most complex regions of the genome, characterized by high gene density and large tracts of segmental duplication. Nearly half of the IGHV region is comprised of segmental duplication segments sharing a high degree of sequence similarity.

The fact that many IGHV genes can occur in 0 to multiple copies has been known for several decades. In fact, greater than half of all known IGHV genes are part of deletion or insertion polymorphisms. Importantly, both IGHV1-69 and IGHV3-30 have long been known to vary in copy number. However, until our recent efforts, given the sequence complexity of the region, the development of reliable and effective high-throughput tools for assaying IGHV alleles and deletion/insertion variants. Thus, the study of these genes in biological phenotypes has been severely limited. Importantly, because of the known paucity of genomic data in the region, next-gen sequence technologies, as well as SNP and CNV arrays are unable to effectively interrogate these extremely important genes.

Applying the IGH Capture/Genotyping Method to Clinical Samples

Cohort of seasonal influenza vaccinees obtained from DFCI (Marasco Lab). Blood draws at Day “0” (pre-vaccination), and Days “7” and “30” (post-vaccination). Samples have undergone antibody repertoire sequencing for all three time points (IgM and IgG). Serum Ab titres for 8 different influenza strains have also been collected at all three time points

Study N=60 samples (additional samples have been collected to extend study)

Genotyping on all 60 completed by mid-December (42 should be completed in 1-2 wks)

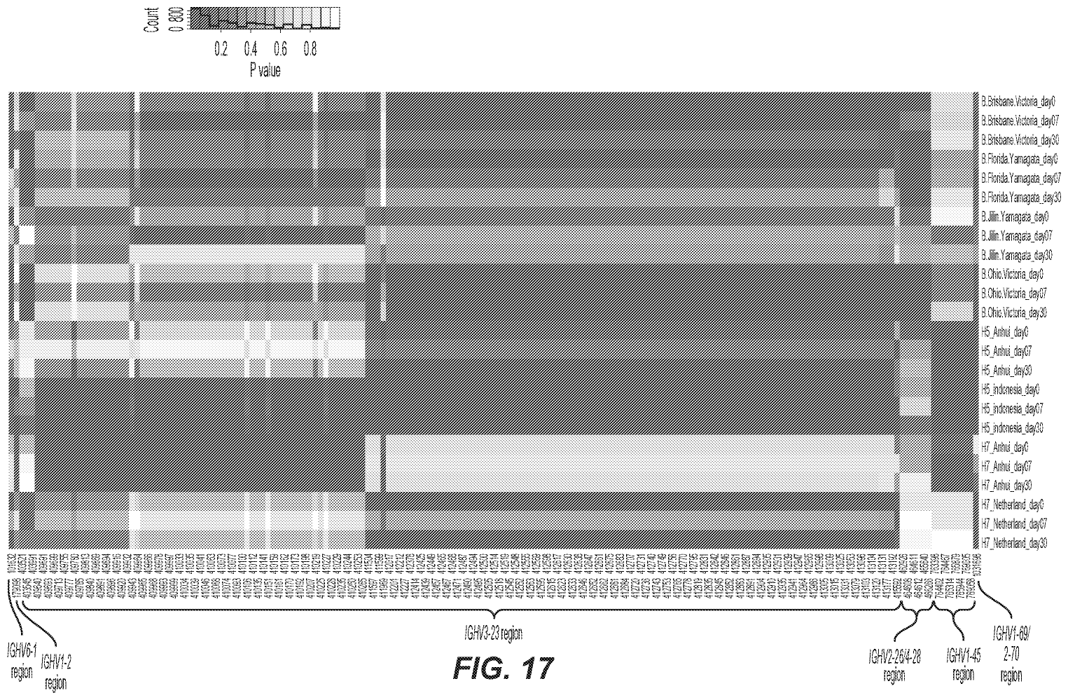

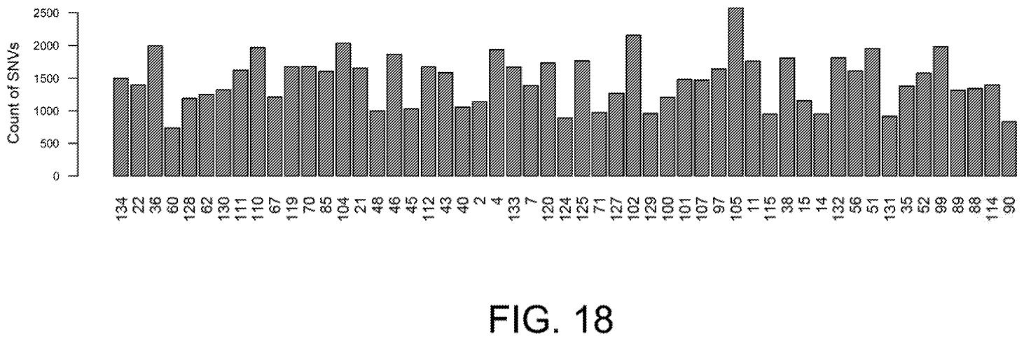

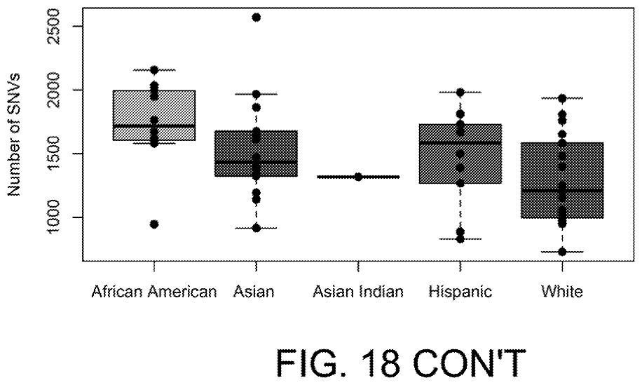

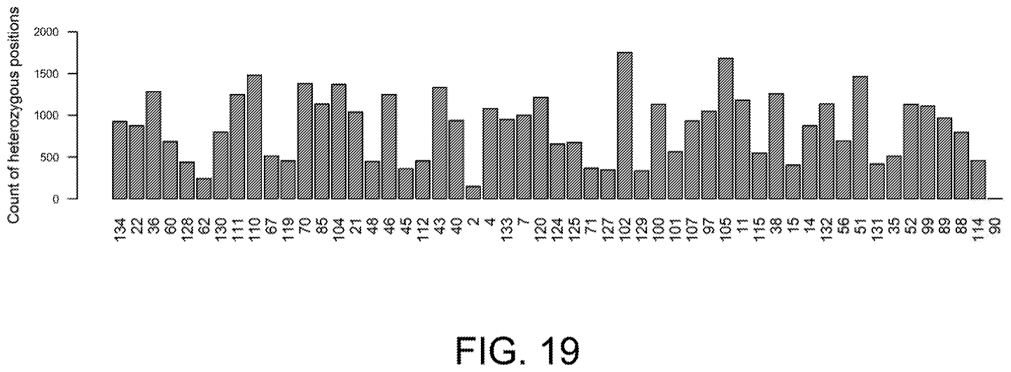

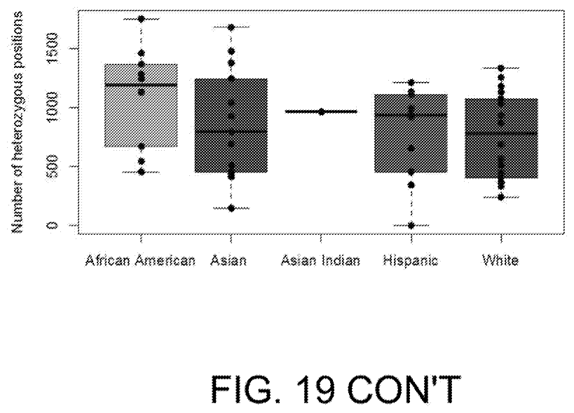

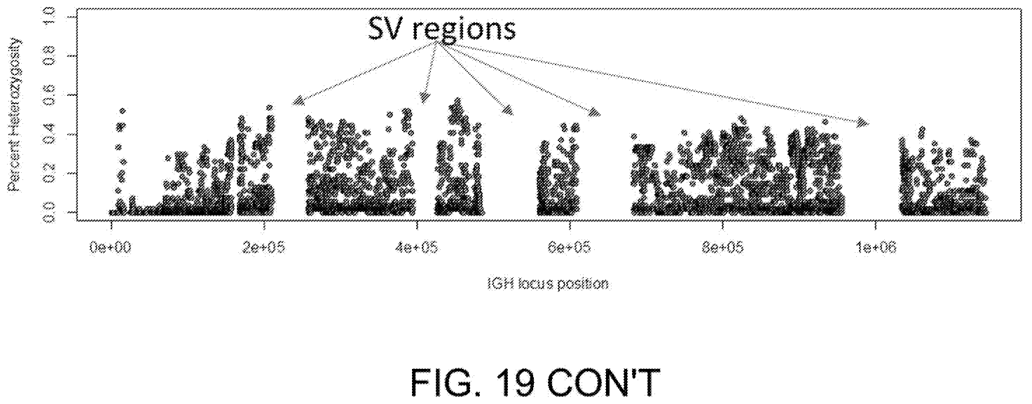

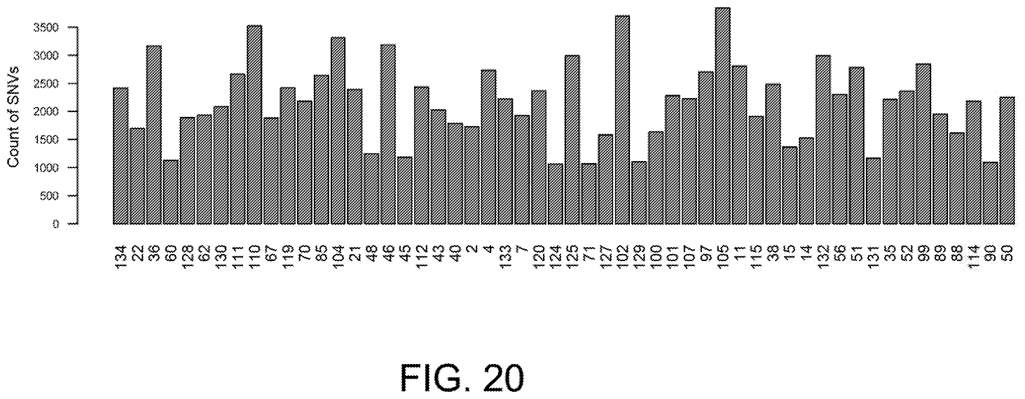

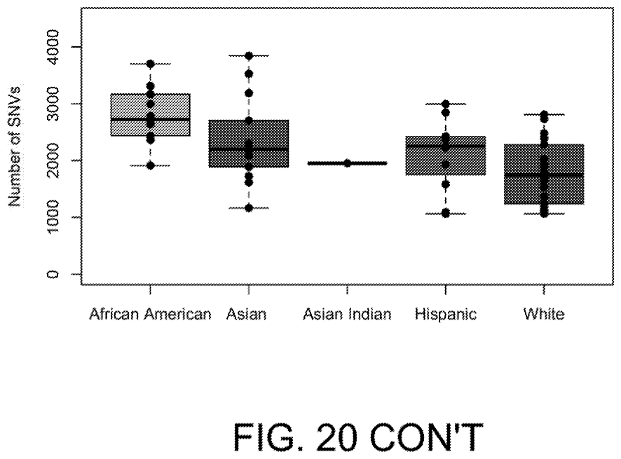

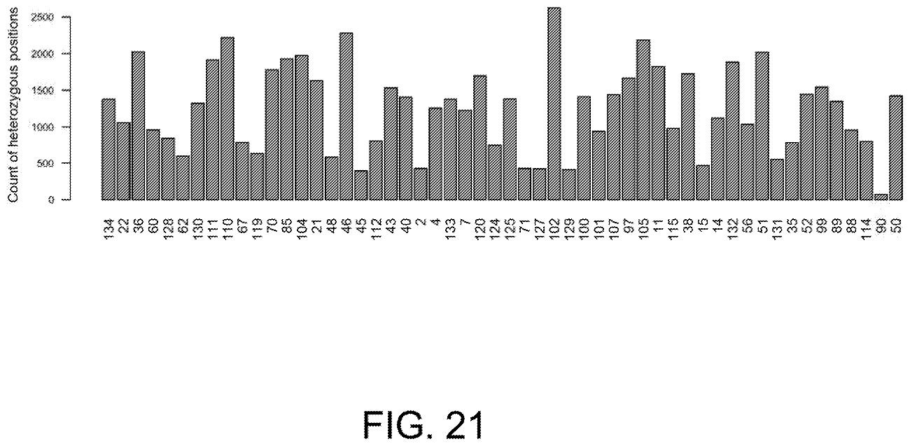

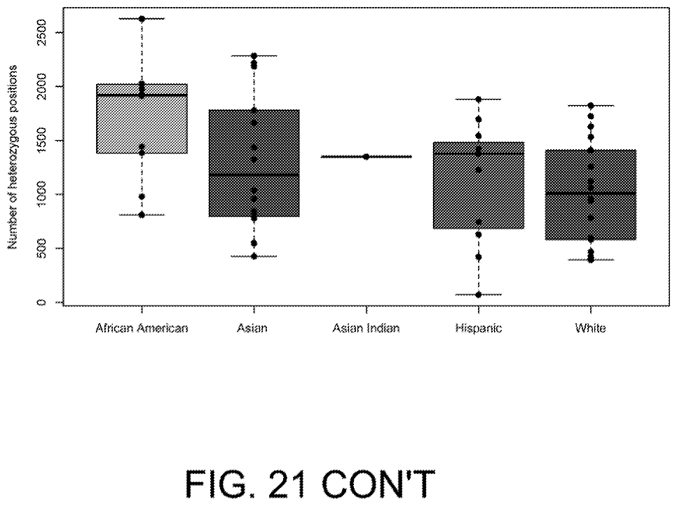

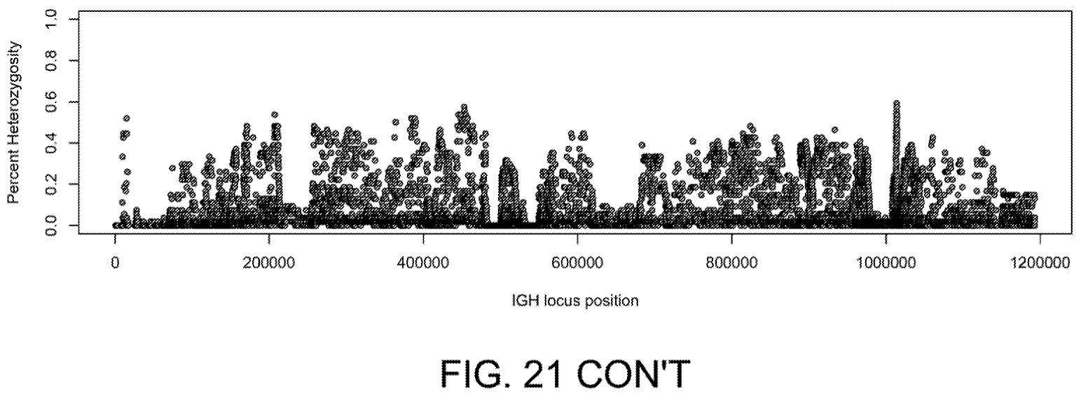

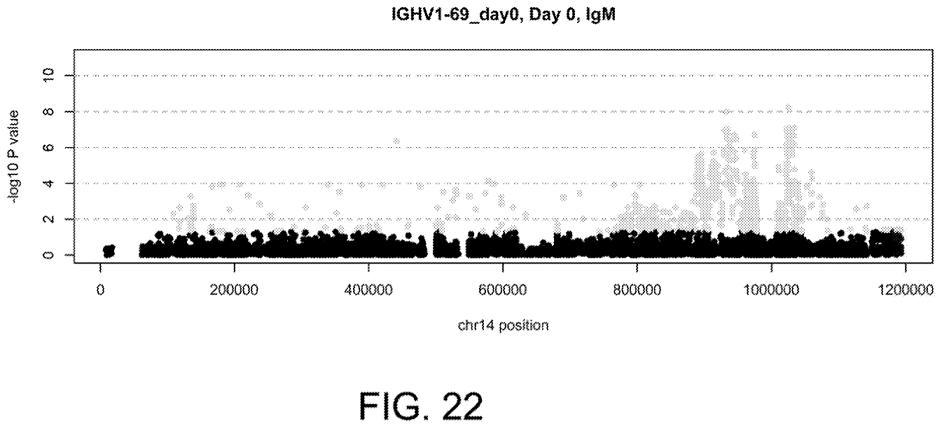

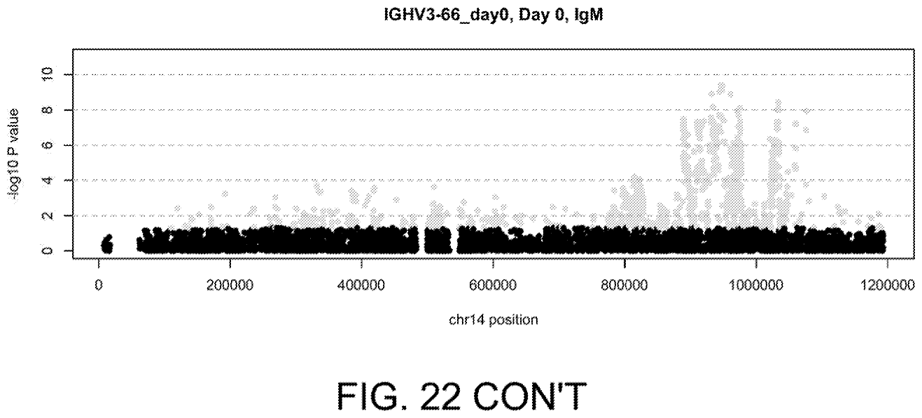

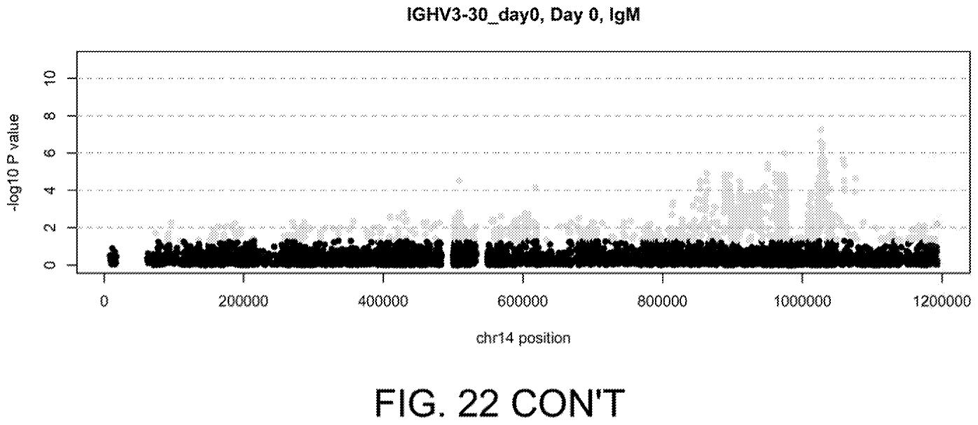

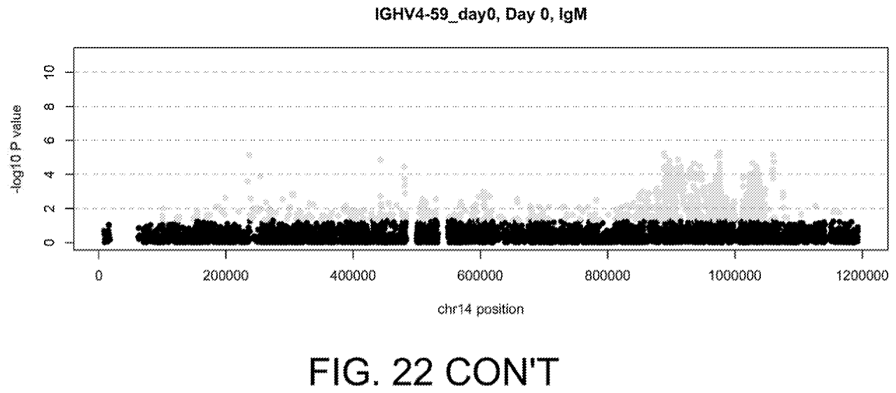

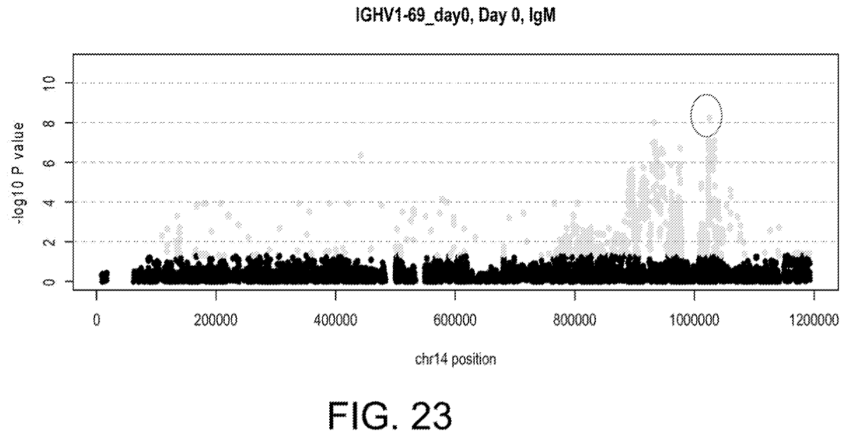

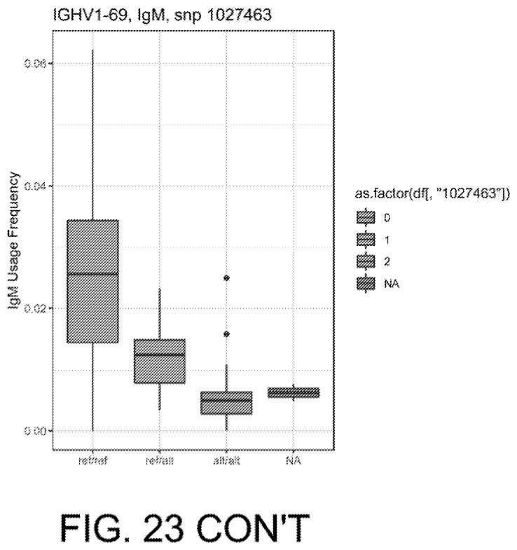

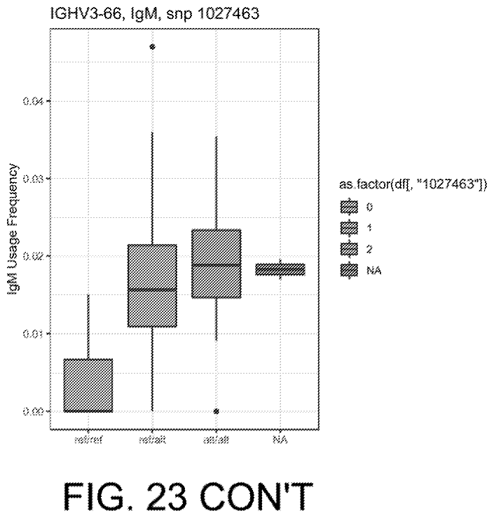

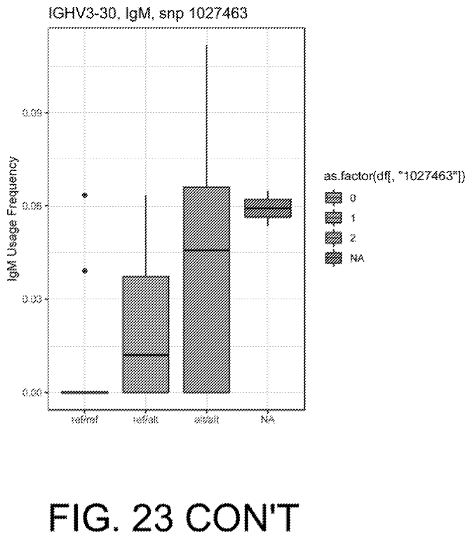

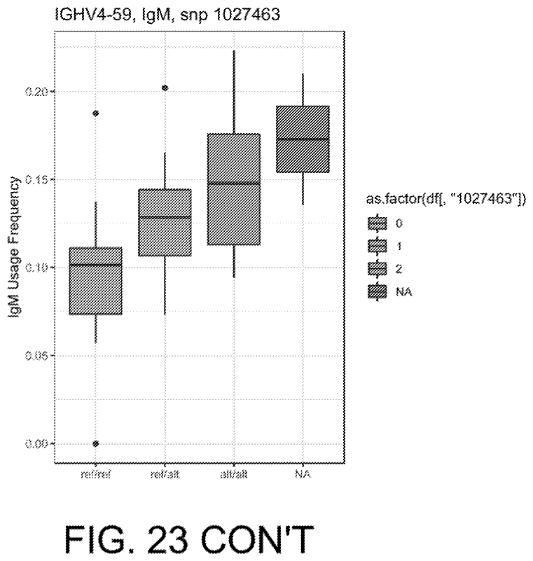

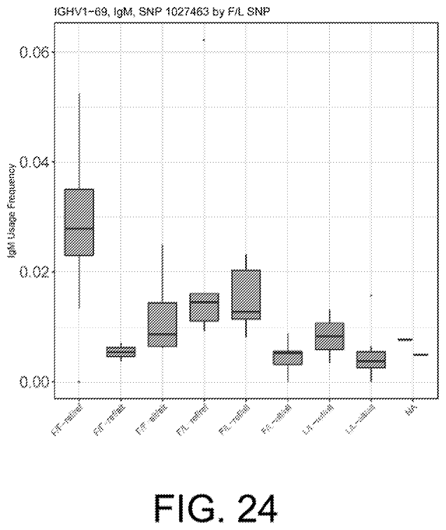

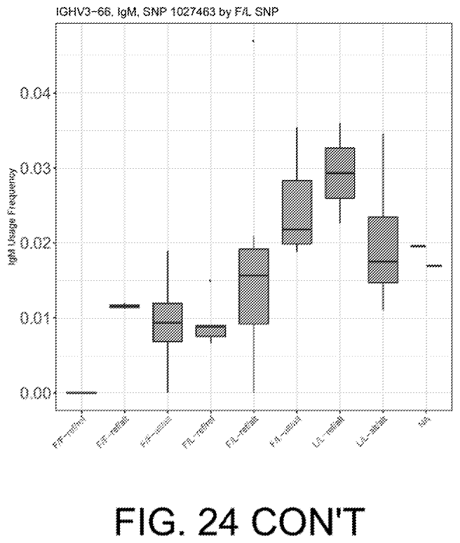

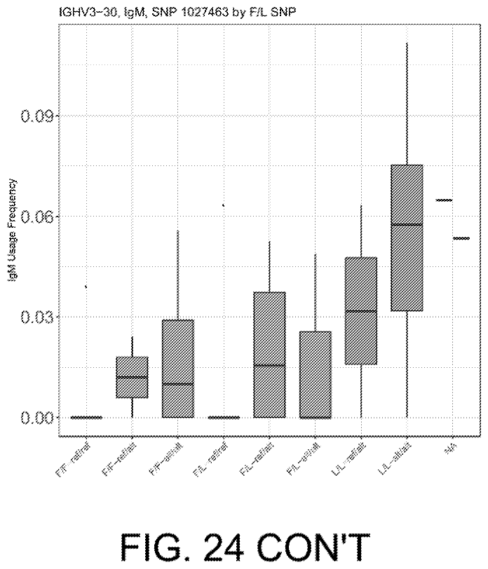

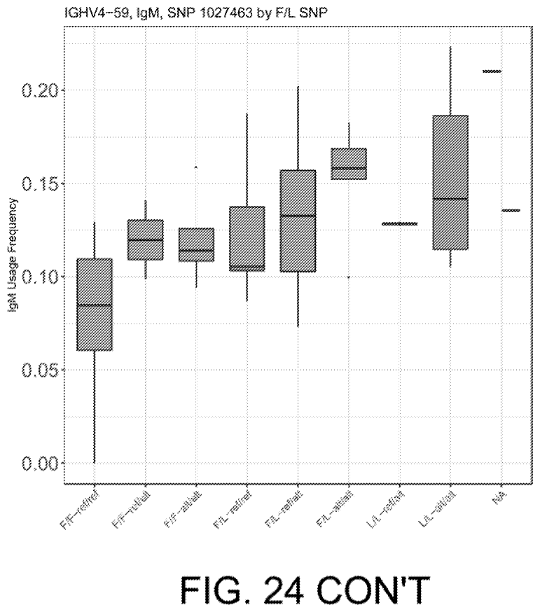

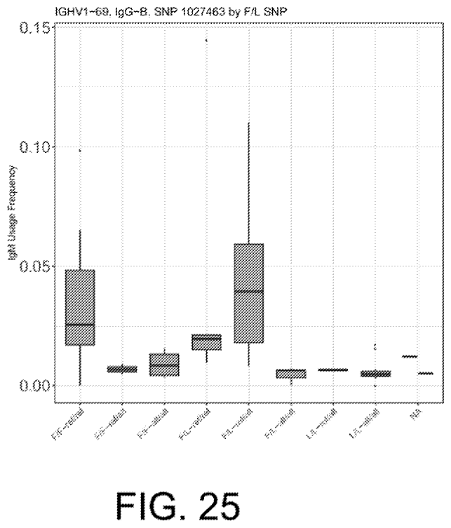

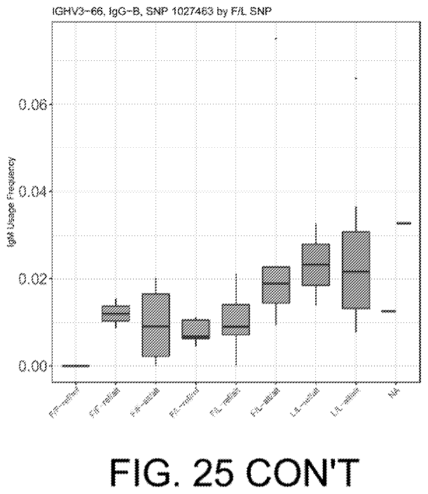

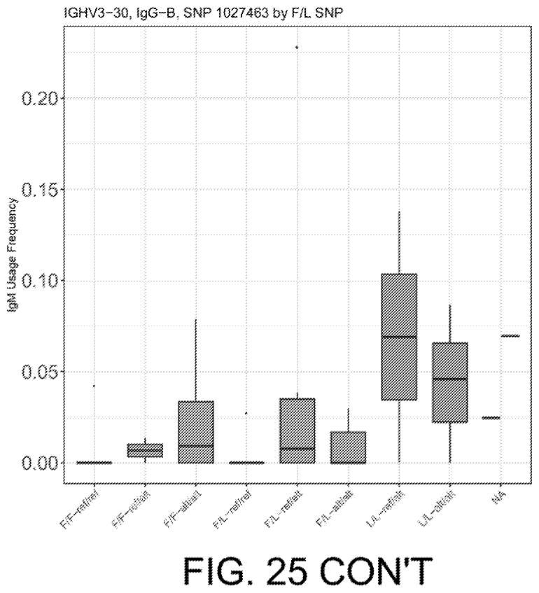

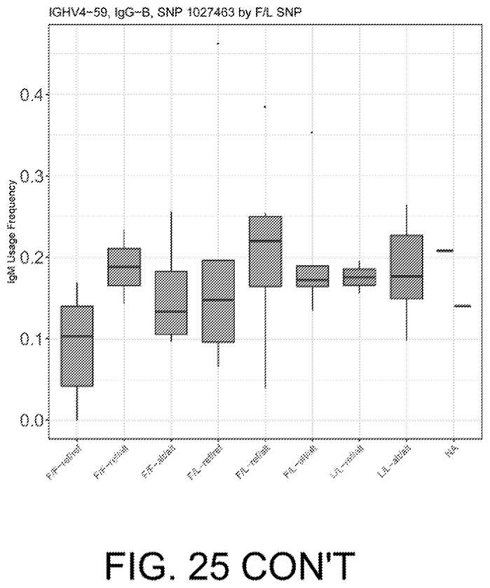

Results. Using first set of samples (n=18), there are 184 SNPs that associate with at least one strain/time point (p<0.0001). These 184 SNPs are shown in the heat map (FIG. 17), ordered by position on chromosome. The strains are ordered on the y axis, by strain and day. The color of tiles corresponds to association P values for a given SNP and Strain/Time point, with red indicating lower p values. (the lowest P value is 3.891169e-06, for SNP in IGHV3-23 region and B.Ohio.Victoria_day0 titer) Some SNPs appear to associate strongly with titers for some strains but not others. For example, the IGHV1-45 region has associations mainly to H5 and H7 strains.

### Example 10

The example herein describes steps to assemble and characterize locus-wide genetic variation in the immunoglobulin heavy chain locus (IGH):

1. If reads are not in BAM format (e.g., in bax.h5 format), files are converted to BAM using SMRTanalysis [1].

2. The following steps are coded into the software package, MsPAC [2]:
a. Reads are aligned to an in-house reference genome using BLASR [3];
b. Single nucleotide polymorphisms (SNPs) are called using Quiver [4];
c. SNPs are phased using WhatsHap [5] using aligned reads and SNPs called from step 2.b.;
d. Using the MsPAC methodology [2], as described here [6], reads are assigned to either haplotype 1 or 2 (or labelled ambiguous if unassignable) based on phased SNPs, and partitioned as such;
e. Haplotype-partitioned reads from haplotypes 1 and 2 and ambiguous reads are binned into haplotype blocks, based on WhatsHap phased SNP calls, and where there is sufficient coverage;
f. Each block is assembled using Canu [7];
g. Original reads are aligned back to assembled haplotype block contigs (2.f), and error corrected using Quiver [3].

3. For determining IGH gene/allele calls, the assembled contigs are aligned to the reference assembly, gene sequences are extracted from each contig, and gene/allele assignments are made via alignments to the IMGT germline database [8]. Additional local reassembly of reads is also carried out for specific gene loci, as needed.

4. Locus-wide SNPs are called by identifying alignment differences between assembled haplotype contigs and the reference genome assembly.

5. Structural variants (SVs) are called using MsPAC (based on multiple sequence alignment and a hidden Markov model).

6. SNP/SV genotypes and gene/allele call data can be used to assess the impacts on antibody repertoire features and associated clinical phenotypes.

### REFERENCES FOR THIS EXAMPLE 2

[1] https://www.pacb.com/products-and-services/analytical-software/smrt-analysis/

[2] https://bitbucket.org/oscarldmspac

[3] https://bmcbioinformatics.biomedcentral.com/articles/10.1186/1471-2105-13-238

[4] https://github.com/PacificBiosciences/GenomicConsensus

[5] https://www.biorxiv.org/content/early/2016/11/14/085050

[6] https://www.biorxiv.org/content/early/2017/09/23/193144

[7] https://genome.cshlp.org/content/27/5/722

[8] http://www.imgt.org/

### Example 11

PacBio protocol

See appendix.

### Example 12

Elucidating the Role of Immunoglobulin Heavy Chain Locus Polymorphism on Antibody Diversity and Function

Antibodies (Abs) are a critical component of the adaptive immune system. Their main function is to selectively recognize and mediate an immune response to non-self antigens. While many studies have focused on dynamics of the Ab response, little is known about the effect of germline polymorphisms on the generation of the Ab repertoire, especially in the context of disease.

Ab genes that encode the heavy chain of Abs within the immunoglobulin heavy chain (IGH) locus have been shown to exhibit extremely high allelic polymorphism and copy number variation between individuals and populations. Locus complexity characterized by large segmental duplications and repetitive elements has caused IGH to be repetitively ignored by genome-wide studies. We have developed a high-throughput capture protocol in combination with long-read sequencing to assemble and genotype germline IGH genes and non-coding polymorphisms. Assessment on a haploid hydatidiform mole, CHM1, where the complete IGH locus has been characterized at nucleotide resolution showed that we can accurately recapitulate genes/alleles from this sample. Further accuracy and efficacy analysis on 7 diploid samples from the 1000 Genomes Project, again with orthogonal sequencing data available, demonstrated that our method yielded high locus coverage (mean >250×), facilitating accurate assembly of IGH genes/alleles. Full-locus genotyping in these 7 individuals revealed an elevated number of SNPs across IGH. For example, the SNP density in NA12878 was 78.66 SNPs per 10 Kb, a 10-fold increase over that observed genome-wide. With the ability to accurately genotype a large number of polymorphisms and copy number variants (CNV) in the IGH locus, our genotyping assay was applied to 35 samples with available repertoire sequencing data. Using both datasets, an eQTL analysis was performed to assess the effect of polymorphisms on the naïve Ab repertoire. We replicated previous findings, showing that coding polymorphisms within the IGHV1-69 and a large structural variant in the region affected the usage of IGHV1-69 in the naïve Ab repertoire. However, the assay's ability to effectively capture and genotype the whole IGH locus allowed us to identify additional nearby polymorphisms with stronger effects on IGHV1-69 usage, as well as the usage of other neighboring IGHV genes. This demonstrates the resolution of our assay for capturing causal variants within coding and non-coding regulatory regions, including both SNPs and CNVs. We are currently expanding our cohort size to comprehensively assess the effect of polymorphisms locus-wide on variability and diversity of the expressed Ab repertoire.

### Example 13

Characterizing the mechanisms that drive variation in the functional antibody (Ab) response is critical to understanding disease processes, and informing the design of improved therapies and prophylactics. Antibodies (Abs) are the most diverse proteins expressed in humans, encoded by hundreds of repeated, and highly homologous immunoglobulin (IG) heavy and light chain gene segments. The formation of and diversity found within an individual's Ab repertoire is mediated by several complex molecular processes, and can be influenced by many factors, including prior history, health status, age, and genetics. With respect to genetic factors, studies in twins have demonstrated that many features of the Ab repertoire are in fact non-random and heritable. This has also been bolstered by direct evidence showing effects of IG germline variants on both the naïve and antigen-stimulated repertoires, with additional downstream impacts on the capacity of individuals to mount antigen-specific responses. This has coincided with a growing interest in the fact that the human IG loci, in particular the IG heavy chain locus (IGH), are among the most structurally complex and diverse regions of the human genome, characterized by elevated levels of both single nucleotide polymorphisms (SNPs) and gene copy number variants (CNVs). To date, however, there has been little effort to define roles of IGH genetic variation in Ab function in humans, representing a significant gap in basic knowledge.

Our long-term goal is to characterize the functional impacts of IG germline polymorphism, such as in the IGH loci and IGV loci, on the Ab repertoire at the level of the genome, individual, and population, as a means to better understand the functional Ab response associated with disease states and clinical phenotypes. As an example, one objective is to identify a baseline set of IGH germline variants that have robust effects on the circulating Ab repertoire. Based on our data, without wishing to be bound by theory, it will be necessary to assay all variant types across the IGH locus, including SNPs in both coding and non-coding regulatory elements, as well as genic CNVs, as current evidence indicates that all of these will play a role. Critically, recent advances now put answers to this fundamental question in reach: first, high resolution descriptions of dynamic features of naïve and antigen-stimulated Ab repertoires are possible via repertoire sequencing (RepSeq); and second, a combination of long-read sequencing technologies and approaches under development in our labs now allow for targeted high-throughput IGH locus genotyping. Gaining a foundational understanding of the modeling capacity of IGH polymorphism on variation observed in the expressed Ab repertoire will provide insight into the molecular mechanisms underlying repertoire development and inter-individual variability, as well as allow for integration of this information into the assessment of the Ab response in disease contexts, with ability to facilitate more targeted, personalized medicine approaches.

We will accomplish this objective by pursuing the following two specific aims:

Aim 1: Construct the First Comprehensive IG Genotype and Ab Repertoire Sequencing Dataset from a Cohort of Healthy Adult Donors to Examine Population Level Variation.

We will utilize sequence-capture protocols and analysis pipelines to target and sequence IGH polymorphisms in existing cohorts of 200 healthy adults. In addition, we will compile and analyze Ab repertoires representing multiple isotypes from these same donors. We will leverage the complementary strengths of these paired genomic and expression data to further develop our IGH bioinformatics pipelines for improved haplotype assembly and genotyping, as well as RepSeq IGH germline gene assignment. This aim will result in a comprehensive set of genotype calls for locus-wide CNVs and SNPs, identification and annotation of IGH variable (V), diversity (D), and joining (J) genes, alleles, and regulatory region variation, as well as metrics on classical repertoire features from gene/allele usage statistics to patterns of somatic hypermutation (SHM). Together these data will represent the most comprehensive population based collection of paired human IG germline genetic and RepSeq data.

Aim 2. Identify IGH Variants that Impact Signatures in Expressed Ab Repertoires of Healthy Adult Donors.

The role of IGH germline variants in Ab expression and function have yet to be defined. By combining the genotypes and repertoire data collected in Aim 1 for 200 healthy adults, we will conduct the first locus-wide_genetic association analysis to comprehensively screen for functional IGH genomic variants associated with features of the expressed IgM, IgG, IgD, IgA, and IgE repertoires. Given that the naïve repertoire serves as the baseline for initial Ab-mediated responses, we will first establish functional IGH variants that robustly associate with heritable features of IgM Ab repertoires characterized in this cohort, including IGHV-, D-, and J-gene usage frequencies, IGHV, D, and J allele-specific usage, V-D and D-J recombination frequencies, and associated diversity in complementarity-determining region-3 (CDR3). In addition, building on our data, we will also further explore IGH genetic associations with features in the IgG, IgD, IgA, and IgE repertoires, specifically including again, IGH gene/allele/recombination frequencies and CDR3 diversity, as well as signatures associated with class-switching and SHM.

### Example 14

Antibodies (Abs) are a diverse family of proteins expressed by B cells, and are critical components of the adaptive immune system. They are encoded by hundreds of genes at three primary immunoglobulin (IG) gene regions: the IG heavy chain (IGH) locus, and two light chain loci, IG kappa (IGK) and IG lambda (IGL). The IGH locus, in particular, has been demonstrated by us and others to exhibit extreme genetic variability at both the individual and population levels. This extreme variation is characterized by the occurrence of single nucleotide polymorphisms (SNPs), as well as large insertions, deletions, and duplications spanning tens of thousands of kilobases, and resulting in losses or gains of functional genes (copy number variants, CNVs). Given its inherent locus sequence complexity and extreme genetic diversity, IGH remains a difficult genomic region to study, thus, little is known about the effects of IGH genetic polymorphism on the function of Abs, and the associated effects on disease pathologies and treatment outcomes. However, with the advent of high-throughput sequencing approaches for profiling the expressed Ab repertoire, it has become increasingly clear that IGH genetic variants, including coding and non-coding SNPs, as well as CNVs, can play a role in the developing Ab response and may contribute to Ab biases observed in many disease contexts. This includes examples in cancer, autoimmunity, infectious disease, and vaccine responsiveness. These data indicate that not all individuals are poised to mount the same Ab response, and that this, at least in part, can be attributed to IGH genetic determinants. With this in mind, the integration of locus-wide IGH population genetic data can inform our understanding of the functional B cell response in disease processes, and help direct better clinical care, such as the design of more effective therapeutic and prophylactic strategies. However, no study to date has sought to comprehensively survey IGH variants locus-wide and identify key polymorphisms contributing to variability in the expressed Ab repertoires of healthy adults. Critically, for such an approach to be successful, new genomic tools are required that are capable of overcoming pitfalls associated with current approaches, and that allow for the comprehensive assaying of IGH variants locus-wide. The example will demonstrate the utility of IGH genotyping methods to comprehensively characterize, for the first time, associations between germline IGH haplotype variation and signatures in expressed antibody repertoires of healthy adult subjects. This example will yield basic insights into the effects of IGH polymorphisms on inter-individual Ab repertoire variation, with implications for the discovery of genomic factors and molecular mechanisms influencing Ab repertoire development and diversity. In addition, this work will lay a foundation for the future integration of IGH genomics into immunological studies seeking to more fully characterize the Ab response in disease and clinical phenotypes.

Individual immune responses are known to track with signatures in the expressed antibody (Ab) repertoire, which we and others have recently demonstrated robustly associate with genetic variants in the immunoglobulin heavy chain locus (IGH); such findings have broad implications. Here, we apply new genomic tools to leverage long-read sequencing for comprehensive IGH genotyping, which we use to characterize IGH variants with impacts on Ab repertoire variability in a multi-ethnic healthy adult population. This example will have outcomes with transformative impacts on B cell immunology and immunogenetics.

Specific Aims of this Example:

Characterizing the mechanisms that drive variation in the functional antibody (Ab) response is important to understanding disease processes, and informing the design of improved therapies and prophylactics. Abs are the most diverse proteins expressed in humans, encoded by 100's of repeated, and highly homologous immunoglobulin (IG) heavy and light chain gene segments. The formation of and diversity found within an individual's Ab repertoire is mediated by several complex molecular processes, and can be influenced by many factors, including prior history, health status, age, and genetics. With respect to genetic factors, studies in twins have demonstrated that many features of the Ab repertoire are in fact non-random and heritable. This has also been bolstered by direct evidence showing effects of IG germline variants on both the naïve and antigen-stimulated repertoires, with additional downstream impacts on the capacity of individuals to mount antigen-specific responses. This has coincided with a growing interest in the fact that the human IG loci, in particular the IG heavy chain locus (IGH), are among the most structurally complex and diverse regions of the human genome, characterized by elevated levels of both single nucleotide polymorphisms (SNPs) and gene copy number variants (CNVs). To date, however, there has been little effort to comprehensively define roles of IGH genetic variation in Ab function in humans, representing a significant gap in basic knowledge.

Our long-term goal is to characterize the functional impacts of IG germline polymorphism on the Ab repertoire at the level of the genome, individual, and population, as a means to better understand the functional Ab response associated with disease states and clinical phenotypes. An objective of this Example is to identify a baseline set of IGH germline variants that have robust effects on the circulating Ab repertoire. Without wishing to be bound by theory, it will be necessary to assay all variant types across the IGH locus, including SNPs in both coding and non-coding regulatory elements, as well as genic CNVs, as current evidence indicates that all of these will play a role. Critically, recent advances now put answers to this fundamental question in reach: first, high resolution descriptions of dynamic features of naïve and antigen-stimulated Ab repertoires are possible via repertoire sequencing (RepSeq); and second, a combination of long-read sequencing technologies and approaches under development in our labs now allow for targeted high-throughput IGH locus genotyping. Without wishing to be bound by theory, gaining a foundational understanding of the modeling capacity of IGH polymorphism on variation observed in the expressed Ab repertoire will provide new insight into the molecular mechanisms underlying repertoire development and inter-individual variability, as well as allow for integration of this information into the assessment of the Ab response in disease contexts, with ability to facilitate more targeted, personalized medicine approaches.

We will pursue the following two specific aims in this Example:

Aim 1: Construct the first comprehensive IG genotype and Ab repertoire sequencing dataset from a cohort of healthy adult donors to examine population level variation. We will utilize new sequence-capture protocols and analysis pipelines to target and sequence IGH polymorphisms in existing cohorts of 200 healthy adults. In addition, we will compile and analyze Ab repertoires representing multiple isotypes from these same donors. We will leverage the complementary strengths of these paired genomic and expression data to validate our IGH bioinformatics pipelines for improved haplotype assembly and genotyping, as well as RepSeq IGH germline gene assignment. This aim will result in a comprehensive set of genotype calls for locus-wide CNVs and SNPs, identification and annotation of IGH variable (V), diversity (D), and joining (J) genes, alleles, and regulatory region variation, as well as metrics on classical repertoire features from gene/allele usage statistics to patterns of somatic hypermutation (SHM). Together these data will represent the most comprehensive population based collection of paired human IG germline genetic and RepSeq data.

Aim 2. Identify IGH variants that impact signatures in expressed Ab repertoires of healthy adult donors. The role of IGH germline variants in Ab expression and function have yet to be defined. By combining the genotypes and repertoire data collected in Aim 1 for 200 healthy adults, we will conduct the first locus-wide genetic association analysis to comprehensively screen for functional IGH genomic variants associated with features of the expressed IgM, IgG, IgD, IgA, and IgE repertoires. Given that the naïve repertoire serves as the baseline for initial Ab-mediated responses, we will first establish functional IGH variants that robustly associate with heritable features of IgM Ab repertoires characterized in this cohort, including IGHV-, D-, and J-gene usage frequencies, IGHV, D, and J allele-specific usage, V-D and D-J recombination frequencies, and associated diversity in complementarity-determining region-3 (CDR3). In addition, we will also further explore IGH genetic associations with features in the IgG, IgD, IgA, and IgE repertoires, specifically including again, IGH gene/allele/recombination frequencies and CDR3 diversity, as well as signatures associated with class-switching and SHM.

Significance:

The immunoglobulin heavy (IGH) and light chain gene regions are the building blocks of antibodies (Abs), critical components of adaptive and innate immunity 1. The IGH locus, specifically, consists of approximately 54 V, 23 D, 6 J, and 9 C functional/open reading frame genes that can contribute to the formation of expressed Abs. Even based on the limited surveys conducted to date, >250 functional IGH alleles are known to occur 2, and this number continues to grow 3-8. The locus is also highly enriched for large copy number variants (CNVs), including deletions, insertions, and duplications of functional genes 9-12,4,13,5, and these show considerable variation with evidence of natural selection among human populations 10,5. This extreme amount of allelic and structural variability has made IGH nearly inaccessible to high-throughput assays, and as a result it has been largely ignored by genome-wide studies 14. This has severely impeded our understanding of the contribution of IGH polymorphism to disease risk, infection and response to vaccines and therapeutics 14,15. Even more fundamentally, in contrast to most genes in the genome, which have been included in expression quantitative trait loci (eQTL) analyses, we know very little about the extent of genetic factors, and thus the associated molecular mechanisms, dictating the regulation of the human Ab response. In fact, the majority of our knowledge regarding specific genomic factors involved in Ab repertoire development and variability comes from animal models 16-18, even though such questions could have greater relevance to human health if addressed in outbred human populations 15.

Although the role of IG germline variants in Ab function was of great interest to the field in earlier decades, it was later superseded by a focus on non-genetic factors and alternative molecular mechanisms used by B cells to create diversity in the repertoire (e.g., somatic hypermutation, SHM). However, evidence continues to accumulate in support of IGH genetic variation being critically important to the human Ab-mediated immune response. First, several studies have shown monozygotic twins are consistent with limited observations implicating IG CNVs and coding/regulatory polymorphisms in inter-individual Ab repertoire variability 9,4,13,22. Second, it is now clear that the Ab response in disease is not simply a random process, as indicated by consistent biases in Ab germline gene usage in various contexts, including cancers, infection, and autoimmune disease 23-26. Furthermore, in many cases, specific IG coding variants have been shown to associate directly with differences in Ab function and binding 23,24,26-30; examples include neutralizing Abs (nAbs) in influenza 31-33, HIV34, and Staphylococcus aureus35. Intriguingly, key functional residues identified in many Abs are polymorphic at the population level, and allele frequencies can vary depending on ethnicity 15,31. Together these findings indicate that, in part due to IG germline variation, not all individuals are genetically poised to mount the same Ab-driven response. Without wishing to be bound by theory, this idea 15 highlights the use of Ab genetic and repertoire signatures in combination to partition populations/cohorts for improved understanding of Ab-mediated responses in disease and directing more tailored care (FIG. 2). However, investigations of the functional effects of human IGH germline variation conducted to date have been limited to only a miniscule fraction of the 1000's of IGH variants known (Refs 5,14, 36,37). This represents a profound knowledge gap, and that a thorough investigation of IGH locus-wide variation is warranted and necessary to begin clarifying the role of IGH polymorphism in the human Ab response.

The work will provide desperately needed gains in our basic understanding of Ab diversity and function through the characterization of links between IGH polymorphisms and features in expressed Ab repertoires at the population level. The results generated here can both drive new models centered around the molecular mechanisms and factors involved in human repertoire development and variation, as well as provide a framework for integrating IGH genotyping into research/clinical workflows for improving the interpretation of Ab repertoire data and the B cell response in human health and disease.

Innovation:

In the past decade, use of high-throughput assays, such as microarrays and whole-genome/exome short-read sequencing (WGS) have dominated the genomics field. However, these methods struggle to accurately and comprehensively assay genetic variation in the most complex and repetitive regions of the genome, including the IGH locus 5,14,38. While the application of high-throughput sequencing to profiling expressed Ab repertoires has begun to provide great insight into dynamic features of the Ab repertoire, the lack of equivalent approaches for IG genomic profiling stands as a critical barrier to fully understanding the role of IG genetic variants in Ab variability and function at the population-level 14,15. To overcome shortcomings of these standard methods, we will apply new wet lab and bioinformatics approaches to utilize Pacific Biosciences (PacBio) long-read sequencing for comprehensive IGH genotyping in any sample. Without wishing to be bound by theory, IGH CNVs, and polymorphisms within coding and regulatory regions will strongly influence the Ab repertoire, with a key role in determining an individual's immune response. Our pairing of IGH genomic and Ab RepSeq profiling will allow the first direct tests for connections between locus-wide IGH polymorphisms and Ab repertoire signatures.

Our data indicate that the approaches will be successful, and the outcomes will be relevant in many disease contexts, and will lead to improvements in our understanding of the mechanisms underlying Ab repertoire diversity and function, and ultimately how this information can be used to inform personalized medicine (FIG. 2).

Approach:

Aim 1: Construct the First Comprehensive IG Genotype and Ab Repertoire Sequencing Dataset from a Cohort of Healthy Adult Donors to Examine Population Level Variation.

A lack of effective genomic tools has stunted our ability to screen IGH polymorphisms at the population level 14,15. However, the genomic structure of IGH is well-known to vary considerably between individuals 9-12,39,40, with as many as 37 (˜50%) functional/ORF IGHV and D gene loci varying in copy number, including deletion variants as large as 75 Kb in length 5,39; no CNVs are reported in J loci, but do extend into IGH constant genes 4,41,42. IGH genes also exhibit significant allelic variation, with some genes having >15 known alleles 2,43. This puts IGH diversity on par with other hyper-polymorphic human loci (e.g., HLA) 4, although descriptions of IG population-level diversity lag far behind. Notably, mapping of haplotype diversity in HLA has been critical for understanding its role in evolution, gene regulation, disease risk and therapeutic response 44-47. While early candidate gene approaches also associated IGH variants with disease susceptibility 48-50, few definitive associations have been made in the era of genome-wide association studies (GWAS) and WGS. This is due to technical difficulties caused by IGH locus complexity/diversity 14,38 that hinder out-of-the-box use of standard high-throughput approaches. Indeed, we have shown commercial SNP arrays tend to have low coverage in IGH, and poorly represent IGHV coding variants and CNVs 5,14 (e.g., the Immuno-array BeadChip 51 includes only 5 markers for the entire ˜1 Mb IGHV gene region, which harbors 1000's of SNPs). In addition, IGH complexity also poses problems for mapping of short-read sequence data38. The 1000 Genomes Project (1KGP) 52,53, which aims to characterize all human genome variants using short-read sequencing, flags genotype calls in >25% of IGHV coding sequence. Other more recent targeted genomic IG approaches 54,55,6 using short-reads have also been limited by the number of IG genes that can be genotyped and/or are not designed to assay non-coding SNPs and CNVs; this is true for RepSeq-based inference methods as well 8,7,56,57. Ultimately, to fully define the role of IGH variation in Ab expression, function and disease, many classes of variation, including CNVs, as well as coding and non-coding SNPs will be critical to resolve 4,13,14,58. Given the complex and multi-allelic nature of IGH, it is clear that specialized genotyping methods capable of capturing locus-wide polymorphism at nucleotide resolution will be required to accurately characterize these regions. In this aim, we will use the application of new IGH capture-sequencing approaches, which overcome many limitations of standard methods by leveraging long-read PacBio sequencing.

Data:

Using a new method that leverages PacBio long-read sequencing for comprehensive IGH genotyping, haplotype and population diversity in the human IG loci is being characterized (e.g., see refs 5,31,59 as background). Most recently, we have developed a custom IGH locus capture assay that can be paired with PacBio long-read sequencing (FIG. 26). This assay uses Nimblegen SeqCap probes to pull down ˜1.2 Mb of sequence targets in human IGHV/D/J gene regions, designed from our published haplotype data 5. Our modified protocol results in sequencing libraries of 6-8 kb, ideal for leveraging the strengths of PacBio long-reads for CNV calling and phased SNP genotyping. With these data, we utilize existing and in-house pipelines, such as BLASR60, Quiver61, WhatsHap62, and MsPAC63 to map, partition, and assemble reads, for SNP calling, gene/allele assignment, and CNV detection (FIG. 26). We have tested this assay using gDNA from one haploid and three diploid samples, each individually sequenced on the PacBio RSII. We used the haploid CHM1 cell line for which we had previously sequenced/assembled the complete IGHV/D/J region from BAC5,59, offering an ideal test case. When comparing IGH capture and BAC data 5 from this sample, we found >98% locus coverage, >99.99% concordance in SNP calls, and 100% concordance in IGHV/D/J allele assignments. Read depth analysis using our custom in-house IGH assembly also revealed the presence of CNVs in this sample (FIG. 26). In the three diploid samples, we also observed ample read coverage of IGH with a mean per base coverage of 243×, collectively covering 100% of IGH V, D, and J genes. Again, CNVs were observable based on read depth profiles and event breakpoint-spanning reads (FIG. 26). In addition, we used MsPAC63 to create haplotype-partitioned assemblies for allele resolution genotyping of IGHV/D/J genes and flanking non-coding regions (FIG. 26). These results demonstrate that this assay is a feasible approach for comprehensive IGH genotyping.

Aim 1.1. Analyze and Assemble a Collection of IgM and IgG Repertoire Feature Statistics in 200 Healthy Adult Donors.

We will compile Ab repertoire data from 200 healthy adults from two cohorts collected by the Dana-Farber Cancer Institute (cohort 1, n=100; M-PI Marasco) and the Stanford University Medical Center (cohort 2, n=100; ). Combined, the cohorts are equally split by gender, and represent a range of ages (18-87 yrs) and ethnicities (projections based on self-reported data: African American, 8%; Asian, 21%; Hispanic, 11%; Caucasian, 60%). Isotype-level RepSeq (cohort 1—IgM, IgG; cohort 2—IgM, IgG, IgA, IgE, IgD) has already been conducted from PBMC cDNA for 160 of 200 donors (n=60 cohort 1; n=100 cohort 2) by targeted IG amplicon sequencing from cDNA using established protocols 31,64,65; average reads per sample are ˜270K. For this aim, we will first conduct IgM and IgG RepSeq in the remaining 40 samples from cohort 1 using the same protocol used for the existing 60 samples (M-PI Marasco; see also Aim 2 Data). Once generated, we will process all data across the two cohorts using the Immcantation pipeline 66,67. A combination of IgDiscover 8, TIgGER 7, haplotype inference 13,57, and direct genotyping (Aims 1.2 & 1.3) will be used to define per sample germline gene/allele assignments. From these data, we will generate metrics on features of the Ab repertoire, such as: (1) IGHV-, D-, and J-gene usage frequencies, (2) IGHV, D, and J allele-specific usage, (3) V-D and D-J recombination frequencies, (4) CDR3 diversity, (5) per gene ratios of IgG, IgA, IgE, and IgD to IgM gene usage (class switch frequency), and (6) per gene SHM frequencies/patterns. These features have been shown to exhibit inter-individual variation, including evidence of germline contributions 19-21,68.

Aim 1.2. Conduct IGH Locus Genotyping in 200 Healthy Adults Leveraging Long-Read Sequencing.

We will undertake comprehensive IGH genotyping in genomic DNA from cohorts 1 and 2 above. For cohort 1, we will utilize our existing capture assay (Studies) to fully sequence the IGH V, D, and J regions from genomic DNA of 100 healthy adult donors. Following this approach, IGH full-locus sequencing libraries will be made for each donor and sequenced individually using the PacBio RSII (Co-I Laird Smith). In addition, to extend our genomic screening in a more cost-effective manner, we will iterate on our IGH full-locus method, and design a second capture panel including only sequence targets within IGHV/D/J coding regions and adjacent flanking regions (±1 Kb). Although this will result in a reduction in the fraction of the locus covered, it will limit our required sequencing space, and allow for use of multiplexed barcoding protocols 69-71 to expand our genotyping effort to a larger number of samples, while still targeting regions of IGH that harbor many functional variants. For example, in our diploid sample results in Data, we genotyped an average of 649 IGH SNPs in IGHV/D/J±1 Kb regions. We will first test this on 12 samples from Aim 1.1, to ensure concordance in allele calls between the two capture panel designs. We will then expand targeted IGHV/D/J germline sequencing/genotyping to the 100 healthy adult donors in cohort 2, utilizing increased sequence throughput of the Sequel platform. Using our newly developed pipelines, we will genotype SNPs and CNVs (inferred using read-depth, CNV region-specific SNP information, local haplotype-assembly, and event breakpoint junction analysis). We will use SNPs in IGHV/D/J genes and flanking canonical regulatory regions (e.g., RSSs, promoters) to perform local allele-specific assemblies to make phased germline gene/allele calls (see FIG. 2). We will also supplement genotyping panels with 12-15 targeted PCR-based assays for CNV calling and additional cross-validations between methods, which we have demonstrated use of previously (Aim 2, Data) 5,31. Lastly, we will further develop our analysis pipeline to integrate RepSeq gene/haplotype inference data for improved genomic haplotype/variant phasing. In both cases, capture designs will include targets for genotyping published ancestry-informative SNPs for ancestry inference (East Asian, African, Caucasian, Hispanic) 72 and chromosome X/Y SNPs for gender assignment73.

Aim 1.3. Population Survey of Paired IGH Genetic Diversity and Ab Repertoire Variability.

With data generated in Aims 1.1/1.2, we will for the first time compile paired population-level IGH genomic and repertoire metrics from the same samples. We will partition cohorts 1 and 2 by ethnicity to generate metrics for SNP, CNV and IGH gene allele frequencies, and compare allele frequencies between ancestry groups using Fₛₜ. Additionally, we will compare general diversity indices (e.g., per gene allelic richness; coding vs. non-coding diversity) and estimates of linkage disequilibrium (LD). Basic IgM/IgG/IgA/IgE/IgD repertoire features compiled in Aim 1.1 will also be compared between populations, offering the ability to assess broader population-specific signatures (e.g., lower or higher total repertoire diversity and/or gene usage variability, etc.).

Without wishing to be bound by theory, this aim will result in the most comprehensive compilation of samples with paired IGH genotype and Ab RepSeq data. While these data will primarily serve as our basis for establishing connections between germline variants and Ab repertoire features in Aim 2, there are additional outcomes that will also result. For one, given the size of the cohort screened, and observations from Data, many new genetic variants can be uncovered. The IGH allele database, ImMunoGeneTics Information System (IMGT; www.imgt.org)43, is a key resource for the immunogenetics community, and known to be incomplete 3-6,14. All RepSeq and variant data generated in Aim1 will be submitted to public repositories, including new IGH alleles, which will be submitted to IMGT. More generally, data generated here will collectively allow for the first accurate population-level views of locus-wide IGH genetic and repertoire variation, which will serve as useful exploratory datasets for the community for generating new models. For example, based on previous investigations of IGH variation among human populations including ours (refs. 5,31), ethnicity-specific signatures can be uncovered in this cohort. Finally, this aim will offer demonstration of the application of our new IGH genotyping protocols/pipelines. These will be made publically available for broad use by the research/clinical community.

Alternatives: Given our experience with Ab RepSeq analysis and unique expertise in IG genomics, the objectives discussed herein can be successful. However, as with any genotyping approach in IGH, there could be hurdles to overcome. For example, despite extensive sequence coverage of IGHV/D/J, we observed low coverage in a few (intergenic) regions; this did not affect gene/allele calling, but could ultimately impact full-locus phasing methods. Analysis revealed dropout was associated with low probe coverage and/or repetitive sequences. We will mitigate this by “boosting” with additional probes flanking these regions, aiming to recruit more long reads to span these low probe coverage areas. While such issues may result in a small proportion of missing genotypes, Data show that a majority of the IGH locus is amenable to genotyping. Furthermore, our ability to cross-validate capture data with targeted PCR and RepSeq data will increase our likelihood for robust genotyping in the majority of samples. Due to the cost required for our full-locus assay, we will adopt a streamlined design for screening cohort 2. While less comprehensive, as noted above, it will still allow for far greater genotyping capacity than other current approaches, and may prove to be a cheaper alternative for broader adoption by interested researchers. Nonetheless, early in the course of the project period, we will also explore sample multiplexing options for our IGH full-locus design. If successful, we will consider expanding our full-locus genotyping into cohort 2 for greater genotyping coverage at lower cost. Also during the course of the project, we may evaluate alternative long-read (e.g., Oxford Nanopore) and phased linked-read methods (e.g., TruSeq/Moleculo, 10× Chromium)74; however, presently, due to costs and other caveats, these methods are not on par with our strategy. Finally, on the informatics side, will continually explore alternative PacBio assembly and genotyping algorithms as they become available.

Aim 2. Identify IGH Variants that Impact Signatures in Expressed Ab Repertoires of Healthy Adult Donors.

There is now strong support for the importance of germline IGH polymorphism in determining the naïve and Ag-stimulated Ab repertoire. Early work in MZ twins provided initial evidence that the Ab repertoire was under genetic control 75. With the advent of high-throughput deep repertoire sequencing, this has now been investigated at greater resolution. Several recent studies of Ab repertoire data in MZ twin pairs revealed that IGHV, D, and J-Contact gene usage, as well as CDR features in naïve repertoires were much more highly correlated between genetically identical twins than between unrelated individuals 19-21. Intriguingly, signatures in Ag-experienced repertoires partly reflected those observed in the naïve, indicating that although memory B cell populations are affected by environmental exposures, they represent sampling events from fairly static, genetically-determined naïve repertoires19-21. Analyses of repertoires in unrelated individuals have also demonstrated that DJ pairing frequencies are not random; by inferring IGHD-J “haplotypes”, it was shown that individuals carrying deletions of particular IGHD genes had more similar D-J recombination patterns 41. Additional examples directly linking IG polymorphisms to IG gene repertoire features also exist, revealing effects of CNVs, and SNPs within IG coding and regulatory regions (Data) 9,31,58,76,77,33,32, including those with relevance to disease and clinical phenotypes 31,58,33,32. However, all studies conducted to date have been based on limited data, restricted by the number of IG variants tested, cohort size, and/or the use of crude measurements of IG gene usage estimated by methods other than direct Ab RepSeq 77; thus, comprehensive investigation of IG germline effects on the Ab repertoire is warranted.

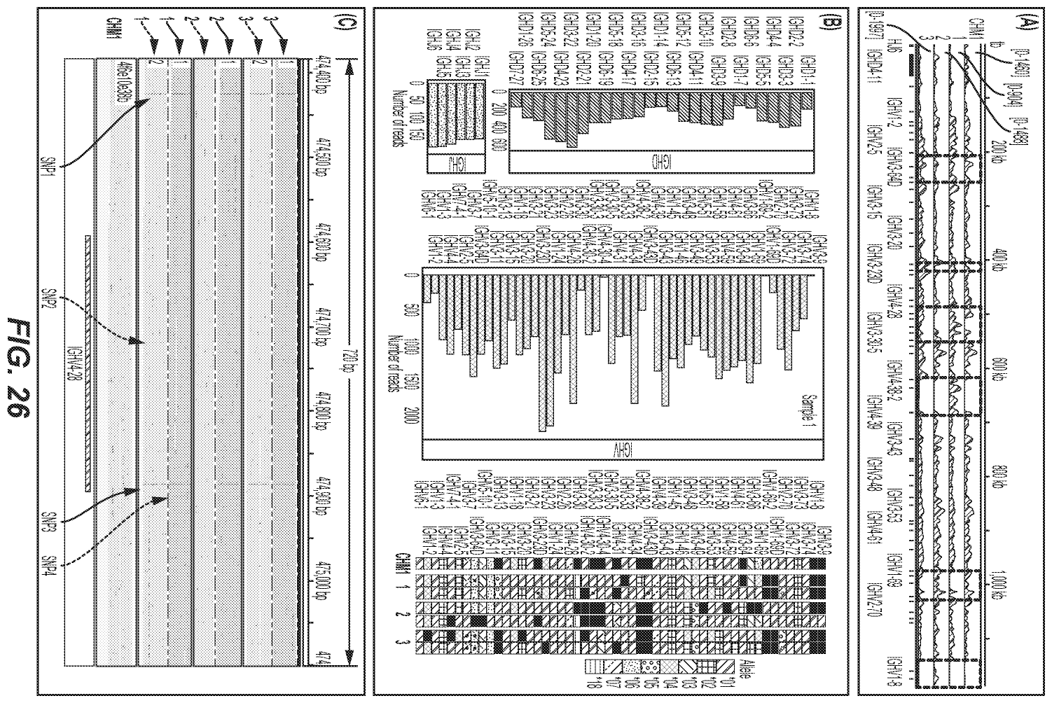

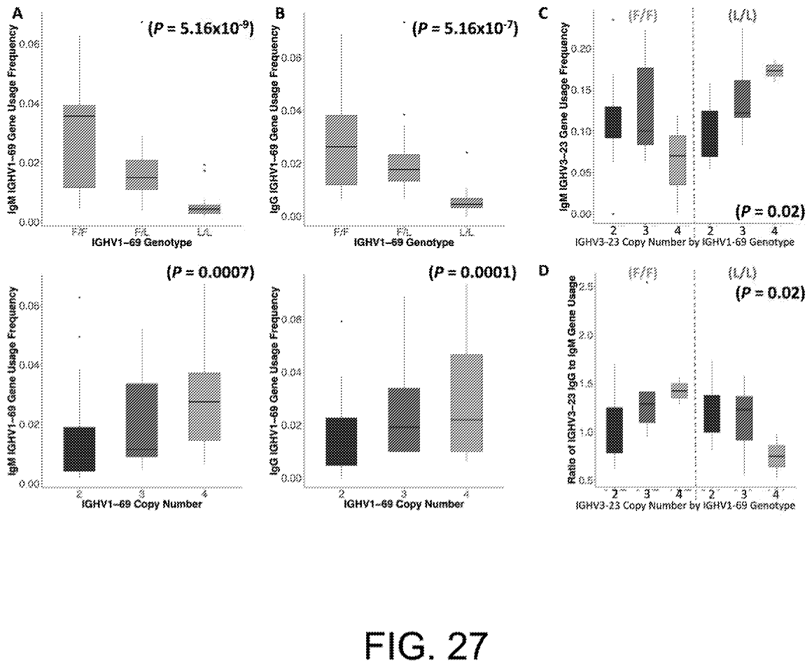

Data: Examples of allelic and copy number variation associating with features in the expressed Ab repertoire. We have begun to explore direct connections between IGH polymorphisms and Ab repertoire variation in detail at several loci. We provide examples from IGHV1-69 and IGHV3-23 here as examples to motivate the work described herein. Both of these genes are characterized by CNV (FIG. 26) and allelic variation. Individuals can carry 2-4 copies of IGHV1-69, and more than 15 alleles are known, subdivided in two groups defined by SNP rs55891010 encoding either a CDR-H2 F54 or L54. Importantly, the CDR-H2 F54 substitution has fundamental function in influenza HA stem-binding23,78, and IGHV1-69 variants have also been implicated in cancer 79,80 and autoimmunity 81,82. Previously 31, we genotyped IGHV1-69 for CDR-H2 F54/L54 alleles and CNV in 18 individuals with accompanying Ab repertoire data. Even in this modestly sized cohort, we found robust connections between this IGHV1-69 SNP, CNV, and repertoire gene usage in both IgM and IgG repertoires. Individuals lacking F54 alleles also had higher ratios of IGHV1-69 IgG clones compared to IgM, with altered levels of SHM. Intriguingly, we also found surprising long-range effects of IGHV1-69 genotype on the usage of genes over 200 Kb away, including IGHV3-30 and IGHV 3-23, which exhibited contrasting genotype-associated patterns from IGHV1-69 in both IgM and IgG subsets 31; both of these genes also exhibit allelic variation and CNV 5. To replicate our findings and further demonstrate the presence of IGH-eQTLs, we have also conducted targeted IGHV1-69 and IGHV3-23 genotyping in 60 individuals of cohort 1 (Aim 1). Again, we observed significant effects of IGHV1-69 genotype/CNV on IgM and IgG gene usage (FIG. 27). Given our previous results 31, we next tested for an effect of IGHV3-23 germline copy number after conditioning on IGHV1-69 genotype (FIG. 27), revealing a significant interaction (opposing effects) of these combined genotypes on IgM IGHV3-23 gene usage. Exploring this further, we also noted differences in the IgG repertoire, demonstrated by assessing the relative ratios of IgG/IgM IGHV 3-23 usage frequencies based on genotype (FIG. 27); such ratios have previously been shown to have underlying genetic components, and suggested to reflect the recruitment of particular genes to memory 19. Together, this work demonstrates clear links between IGH genotype, repertoire, and the functional Ab response, and that genotype information can be useful for providing a more detailed understanding of the Ab response.

Aim 2.1. Characterizing Functional IGH Germline Variants with Effects on Baseline Ab Repertoires of Healthy Adults.

Here, we will directly investigate effects of IGH polymorphism on baseline Ab repertoire features by utilizing the IGH genotypes and paired RepSeq data in 200 adults (cohorts 1 & 2; Aim 1) to perform cis-eQTL analyses (“cis” referring to variants within IGH). Analyses will be performed using a combination of the matrix-eQTL R package83 and PLINK84, which implement generalized linear model (GLM) and/or ANOVA frameworks, allowing for testing for additive and dominant effects, and interaction terms. IGH genotypes will be used as modeling variables, and the 6 repertoire features compiled in Aim 1.1 as quantitative traits. Analyses will be conducted in multiple stages to account for differences in the genotyping assay design used. First, we will conduct a cis-eQTL analysis using IGH full-locus genotypes and repertoire data in cohort 1, allowing for a complete locus-wide screen for functional variants associated with variability in IgM and IgG repertoire features. Second, as additional isotypes are represented in the RepSeq data available for cohort 2, we will conduct a secondary analysis for IgM, IgG, IgA, IgE, and IgD features. Finally, for increased statistical power, we will conduct a combined analysis in all 200 individuals (cohorts 1 & 2), by considering overlapping genotypes assayed by both capture panel designs, and targeted CNV PCR-based genotyping.

To ensure robustness and account for relevant covariates in our analyses, eQTL models will incorporate gender, ethnicity, and cohort (i.e., 1 or 2). For gene usage/expression, we will also employ PEER 85 to assess the presence of any additional hidden covariates (e.g., batch/technical effects, or unknown environmental variables); this application will not be applicable for all repertoire features to be tested (e.g., SHM). PEER can estimate hidden covariates, as well as their weight, subtract these, and produce a residual matrix that can be used for association analysis. In standard RNA-seq, it has been shown to reduce false-positive associations, and improve statistical power by reducing noise. A false discovery rate will be used to control for multiple testing 83,86. In addition to individual cis-eQTLs, we will look for gene-gene interaction effects, and long-range haplotype effects (Data). Given we have previously identified combined effects of IGH gene CNV and allelic variants (Data) 9,31, we will perform tests in CNV regions for effects of copy number changes of particular alleles. In addition, we will look for interactions between age and genotype, using an interaction term in a separate GLM analysis (exact ages are known for 140/200 samples). Although analyses combined across all samples in our cohort will have the most power, we will also test for eQTLs independently within each ethnic background of cohort 1 and 2, allowing for comparisons between African Americans, Asians, Hispanics, and Caucasians.

Projected Outcomes: Nearly four decades since the study of IG genetics began, the role of human IGH germline variants in Ab expression and function have yet to be comprehensively defined. Our analysis will result in the first catalogue of functional IGH variants associated with features of the Ab repertoire. These results will be useful to a growing community of immunologists using Ab repertoire sequencing. Given that the primary variants identified in this aim are those associated with baseline repertoire features (e.g., gene usage), this catalogue could provide useful a priori information for initial studies of IGH germline repertoire effects in other disease contexts of interest; especially considering that we and others have shown that IGH variants impacting the naïve repertoire can also have associations with other key signatures in Ag-stimulated repertoires associated with disease and clinical phenotypes 31,33,35. On the basic research side, these data will also have implications, as much remains to be learned about molecular mechanisms and factors involved in human Ab repertoire development and variability. Linking functional information (e.g., eQTLs) back to the rich IGH haplotype data produced in Aim 1, can serve as a useful starting point for delineating such mechanisms, e.g., by highlighting functional sequence motifs and candidate transcription factors involved, or providing insight into broader haplotype effects, such as impacts of large deletions on the IGH epigenetic landscape. This will help direct models that may be testable in either human primary samples and/or animal models (e.g.,16-18).

Alternatives:

Based on our cohort sizes, eQTL analyses will allow for even fairly subtle effects of IGH germline variation on Ab repertoire features, from gene usage to SHM signatures. Power calculations using minor allele frequency (MAF; 0.45) and usage variation of IGHV1-69 as an example, indicate our combined analysis (n=200) has a power of 1 for detecting significant eQTLs; lower MAFs down to 0.05 still have detection power of ˜0.8. After partitioning by ethnicity, power to detect small effects and gene-gene interactions decreases. However, identification of variants with large effect sizes should still be possible. For example, to make this point, by using only the 20 Caucasian samples we have already genotyped at IGHV1-69 in cohort 1 (Data), the SNP and CNV are capable of explaining ˜70% of IGHV1-69 gene usage variation in IgM (P=4.92×10-5; consistent with ref31). Given the resolution at which we will be able to genotype IGH, multiple layers of haplotype information are likely to further improve our power to detect differences. In addition to Ab features for which we have already demonstrated effects of specific germline variants, we will also investigate associations with SHM patterns and biases of V-(D)-J recombination events. Germline effects on SHM patterns have recently been postulated68. A recent study showed also that effects on D-J recombination could be observed after partitioning samples by the presence of IGHD gene deletion haplotype 41; again, using a cohort of only 25 individuals. Given our cohorts are larger, this investigation is worth the effort. Lastly, we will account for and assess the effects of age, which is known to influence the repertoire 87. Although underpowered to detect age-genotype interactions, data indicate that at a minimum our analyses will establish proof-of-principal concepts and direction for future investigations in expanded and more targeted cohorts.

### REFERENCES CITED IN THIS EXAMPLE

1. Murphy K, Travers P, Walport M. Janeway's immunology. Garland science. 2012. PMID: 25182350
2. Pallarès N, Lefebvre S, Matsuda F, Lefranc M. The Human Immunoglobulin Heavy Variable Genes. Exp Clin Immunogenet. 1999; 16(1):36-60. PMID: 10087405
3. Wang Y, Jackson K J L, Sewell W a, Collins A M. Many human immunoglobulin heavy-chain IGHV genepolymorphisms have been reported in error. Immunol Cell Biol. 2008; 86(2):111-5. PMID: 18040280
4. Boyd S D, Gaëta B a, Jackson K J, Fire A Z, Marshall E L, Merker J D, Maniar J M, Zhang L N, Sahaf B, Jones C D, Simen B B, Hanczaruk B, Nguyen K D, Nadeau K C, Egholm M, Miklos D B, Zehnder J L, Collins A M. Individual variation in the germline Ig gene repertoire inferred from variable region generearrangements. J Immunol. 2010; 184(12):6986-6992. PMID: 20495067
5. Watson C T, Steinberg K M, Huddleston J, Warren R L, Malig M, Schein J, Willsey a J, Joy J B, Scott J K, Graves T a, Wilson R K, Holt R a, Eichler E E, Breden F. Complete haplotype sequence of the human immunoglobulin heavy-chain variable, diversity, and joining genes and characterization of allelic and copy-number variation. Am J Hum Genet. 2013 Apr. 4; 92(4):530-46. PMCID: PMC3617388
6. Scheepers C, Shrestha R K, Lambson B E, Jackson K J L, Wright I a, Naicker D, Goosen M, Berrie L, Ismail A, Garrett N, Abdool Karim Q, Abdool Karim S S, Moore P L, Travers S a, Morris L. Ability To Develop Broadly Neutralizing HIV-1 Antibodies Is Not Restricted by the Germline Ig Gene Repertoire. J Immunol. 2015 Mar. 30; 194(9):4371-8. PMID: 25825450
7. Gadala-Maria D, Yaari G, Uduman M, Kleinstein S H. Automated analysis of high-throughput B-cell sequencing data reveals a high frequency of novel immunoglobulin V gene segment alleles. Proc Natl Acad Sci. 2015; 112(8):201417683. PMID: 25675496
8. Corcoran M M, Phad G E, Bernat N V, Stahl-Hennig C, Sumida N, Persson M A A, Martin M, Hedestam G B K. Production of individualized V gene databases reveals high levels of immunoglobulin genetic diversity. Nat Commun. 2016; 7:13642. PMCID: PMC5187446
9. Sasso E H, Johnson T, Kipps T J. Expression of the immunoglobulin VH gene 51p1 is proportional to its germline gene copy number. J Clin Invest. 1996; 97(9):2074-80. PMID: 8621797
10. Sasso E H, Buckner J H, Suzuki L A. Ethnic differences in polymorphism of an immunoglobulin VH3 gene. J Clin Invest. 1995; 96(3):1591-1600. PMID: 7657830
11. Chimge N-O, Pramanik S, Hu G, Lin Y, Gao R, Shen L, Li H. Determination of gene organization in the human IGHV region on single chromosomes. Genes Immun. 2005; 6(3):186-93. PMID: 15744329
12. Pramanik S, Cui X, Wang H-Y, Chimge N-O, Hu G, Shen L, Gao R, Li H. Segmental duplication as one of the driving forces underlying the diversity of the human immunoglobulin heavy chain variable gene region. BMC Genomics. 2011; PMID: 21272357
13. Kidd M J, Chen Z, Wang Y, Jackson KJ, Zhang L, Boyd S D, Fire A Z, Tanaka M M, Gaëta B a, Collins A M. The inference of phased haplotypes for the immunoglobulin H chain V region gene loci by analysis of VDJ gene rearrangements. J Immunol. 2012; 188(3):1333-40. PMID: 22205028
14. Watson C T, Breden F. The immunoglobulin heavy chain locus: genetic variation, missing data, and implications for human disease. Genes Immun. 2012 July; 13(5):363-73. PMID: 22551722
15. Watson C T, Glanville J, Marasco W A. The Individual and Population Genetics of Antibody Immunity. Trends Immunol. 2017; 38(7):459-470. PMCID: PMC5656258
16. Choi N M, Loguercio S, Verma-Gaur J, Degner S C, Torkamani A, Su A I, Oltz E M, Artyomov M, Feeney A J. Deep sequencing of the murine IgH repertoire reveals complex regulation of nonrandom V gene rearrangement frequencies. J Immunol. 2013; 191:2393-402. PMID: 23898036
17. Espinoza C R, Feeney A J. The extent of histone acetylation correlates with the differential rearrangement frequency of individual VH genes in pro-B cells. J Immunol. 2005; 175:6668-6675. PMID:16272322
18. Espinoza C R, Feeney A J. Chromatin accessibility and epigenetic modifications differ between frequently and infrequently rearranging VH genes. Mol Immunol. 2007; 44:2675-2685. PMID: 17218014
19. Glanville J, Kuo T C, von Büdingen H-C, Guey L, Berka J, Sundar P D, Huerta G, Mehta G R, Oksenberg J R, Hauser S L, Cox D R, Rajpal A, Pons J. Naive antibody gene-segment frequencies are heritable and unaltered by chronic lymphocyte ablation. Proc Natl Acad Sci USA. 2011 Dec. 13; 108(50):20066-71.PMID: 22123975
20. Wang C, Liu Y, Cavanagh M M, Le Saux S, Qi Q, Roskin K M, Looney T J, Lee J-Y, Dixit V, Dekker C L, Swan G E, Goronzy J J, Boyd S D. B-cell repertoire responses to varicella-zoster vaccination in human identical twins. Proc Natl Acad Sci U SA. 2015; 112(2):500-5. PMID: 25535378
21. Rubelt F, Bolen C R, Mcguire H M, Heiden J A Vander, Gadala-maria D, Levin M, Euskirchen G M, Mamedov M R, Swan G E, Dekker C L, Cowell L G, Kleinstein S H, Davis M M. Individual heritable differences result in unique Lymphocyte receptor repertoires of naïve and antigen-experienced cells. Nat Commun. 2016; 6:1-12. PMCID: PMC5191574
22. Feeney A J, Atkinson M J, Cowan M J, Escuro G, Lugo G. A defective Vkappa A2 allele in Navajos which may play a role in increased susceptibility to haemophilus influenzae type b disease. J Clin Invest. 1996; 97(10):2277-2282. PMID: 8636407
23. Sui J, Hwang W C, Perez S, Wei G, Aird D, Chen L, Santelli E, Stec B, Cadwell G, Ali M, Wan H, Murakami A, Yammanuru A, Han T, Cox N J, Bankston L A, Donis R O, Liddington R C, Marasco W A. Structural and functional bases for broad-spectrum neutralization of avian and human influenza A viruses. Nat Struct Mol Biol. 2009; 16(3):265-273. PMCID: PMC2692245
24. Williams W B, Liao H-X, Moody M A, Kepler T B, Alam S M, Gao F, Wiehe K, Trama A M, Jones K, Zhang R, Song H, Marshall D J, Whitesides J F, Sawatzki K, Hua A, Liu P, Tay M Z, Seaton K E, Shen X, Foulger A, Lloyd K E, Parks R, Pollara J, Ferrari G, Yu J-S, Vandergrift N, Montefiori D C, Sobieszczyk M E, Hammer S, Karuna S, Gilbert P, Grove D, Grunenberg N, McElrath M J, Mascola J R, Koup R A, Corey L, Nabel G J, Morgan C, Churchyard G, Maenza J, Keefer M, Graham B S, Baden L R, Tomaras G D, Haynes B F. Diversion of HIV-1 vaccine-induced immunity by gp41-microbiota cross-reactive antibodies. Science (80-). 2015; 349(6249). PMID: 1000111945
25. Foreman A L, Van de Water J, Gougeon M L, Gershwin M E. B cells in autoimmune diseases: Insights from analyses of immunoglobulin variable (Ig V) gene usage. Autoimmun Rev. 2007; 6(6):387-401.PMID: 17537385
26. Zhou T, Zhu J, Wu X, Moquin S, Zhang B, Acharya P, Georgiev I S, Altae-Tran H, Chuang G Y, Joyce M G, DoKwon Y, Longo N S, Louder M, Luongo T, McKee K, Schramm C A, Skinner J, Yang Y, Yang Z, Zhang Z, Zheng A, Bonsignori M, Haynes B F, Scheid J F, Nussenzweig M C, Simek M, Burton D R, Koff W, Mullikin J C, Connors M, Shapiro L, Nabel G J, Mascola J R, Kwong P D. Multidonor analysis reveals structural elements, genetic determinants, and maturation pathway for HIV-1 neutralization by VRC01-class antibodies. Immunity. 2013; 39(2):245-258. PMID: 23911655
27. Liu L, Lucas A H. IGH V3-23*01 and its allele V3-23*03 differ in their capacity to form the canonical human antibody combining site specific for the capsular polysaccharide of Haemophilus influenzae type b. Immunogenetics. 2003; 55(5):336-338. PMID: 12845501
28. Avnir Y, Tallarico A S, Zhu Q, Bennett A S, Connelly G, Sheehan J, Sui J, Fahmy A, Huang C, Cadwell G, Bankston L A, McGuire A T, Stamatatos L, Wagner G, Liddington R C, Marasco W A. Molecular signatures of hemagglutinin stem-directed heterosubtypic human neutralizing antibodies against influenza A viruses. PLoS Pathog. 2014; 10(5):e1004103. PMCID: PMC4006906
29. Throsby M, van den Brink E, Jongeneelen M, Poon L L M, Alard P, Cornelissen L, Bakker A, Cox F, van Deventer E, Guan Y, Cinatl J, ter Meulen J, Lasters I, Carsetti R, Peiris M, de Kruif J, Goudsmit J. Heterosubtypic neutralizing monoclonal antibodies cross-protective against H5N1 and H1N1 recovered from human IgM+ memory B cells. PLoS One. 2008; 3(12):e3942. PMID: 19079604
30. Kashyap A K, Steel J, Oner A F, Dillon M A, Swale R E, Wall K M, Perry K J, Faynboym A, Ilhan M, Horowitz M, Horowitz L, Palese P, Bhatt R R, Lerner R A. Combinatorial antibody libraries from survivors of the Turkish H5N1 avian influenza outbreak reveal virus neutralization strategies. Proc Natl Acad Sci USA. 2008; 105(16):5986-5991. PMID: 18413603
31. Avnir Y, Watson C T, Glanville J, Peterson E C, Tallarico A S, Bennett A S, Qin K, Fu Y, Huang C-Y, Beigel J H, Breden F, Quan Z, Marasco W A. IGHV1-69 polymorphism modulates anti-influenza antibody repertoires, correlates with IGHV utilization shifts and varies by ethnicity. Sci Rep. 2016; 6:20842.PMCID: PMC4754645
32. Pappas L, Foglierini M, Piccoli L, Kallewaard N L, Turrini F, Silacci C, Fernandez-Rodriguez B, Agatic G, Giacchetto-Sasselli I, Pellicciotta G, Sallusto F, Zhu Q, Vicenzi E, Corti D, Lanzavecchia A. Rapid development of broadly influenza neutralizing antibodies through redundant mutations. Nature. 2014; 516(7531):418-422. PMID: 25296253
33. Wheatley a. K, Whittle J R R, Lingwood D, Kanekiyo M, Yassine H M, Ma S S, Narpala S R, Prabhakaran M S, Matus-Nicodemos R a., Bailer R T, Nabel G J, Graham B S, Ledgerwood J E, Koup R a., McDermotta. B. H5N1 Vaccine-Elicited Memory B Cells Are Genetically Constrained by the IGHV Locus in the Recognition of a Neutralizing Epitope in the Hemagglutinin Stem. J Immunol. 2015; 195(2):602-10.PMID: 26078272
34. Yacoob C, Pancera M, Vigdorovich V, Oliver B G, Glenn J A, Feng J, Sather D N, McGuire A T, Stamatatos L. Differences in Allelic Frequency and CDRH3 Region Limit the Engagement of HIV Env Immunogens by Putative VRC01 Neutralizing Antibody Precursors. Cell Rep. 2016; 17(6):1560-1570.PMID: 27806295
35. Yeung Y A, Foletti D, Deng X, Abdiche Y, Strop P, Glanville J, Pitts S, Lindquist K, Sundar P D, Sirota M, Hasa-Moreno A, Pham A, Melton Witt J, Ni I, Pons J, Shelton D, Rajpal A, Chaparro-Riggers J. Germline-encoded neutralization of a Staphylococcus aureus virulence factor by the human antibody repertoire. Nat Commun. 2016; 7:13376. PMID: 27857134
36. Gibson G, Powell J E, Marigorta U M. Expression quantitative trait locus analysis for ranslational medicine. Genome Med. 2015; 7(1):60. PMID: 26110023
37. Keen J C, Moore H M. Personalized Medicine The Genotype-Tissue Expression (GTEx) Project: Linking Clinical Data with Molecular Analysis to Advance Personalized Medicine. 2015; 22-29. PMCID:PMC4384056
38. Watson C T, Matsen IV F A, Jackson K J L, Bashir A, Laird Smith M, Glanville J, Breden F, Kleinstein S H, Collins A M, Busse C E. Comment on A Database of Human Immune Receptor Alleles Recovered from Population Sequencing Data”. J Immunol. 2017; 198:3371-3373. PMID: 28416712
39. Milner E C, Hufnagle W O, Glas A M, Suzuki I, Alexander C. Polymorphism and utilization of human VH Genes. Ann N Y Acad Sci. 1995; 764:50-61. PMID: 7486575
40. Cook G P, Tomlinson I M, Walter G, Riethman H, Carter N P, Buluwela L, Winter G, Rabbitts T H. A map of the human immunoglobulin VH locus completed by analysis of the telomeric region of chromosome14q. Nat Genet. 1994; 7(2):162-8. PMID: 7920635
41. Kidd M J, Jackson K J L, Boyd S D, Collins A M. DJ Pairing during VDJ Recombination Shows Positional Biases That Vary among Individuals with Differing IGHD Locus Immunogenotypes. J Immunol. 2015; 196(3):1158-64. PMID: 26700767
42. Brusco a, Saviozzi S, Cinque F, Bottaro a, DeMarchi M. A recurrent breakpoint in the most common deletion of the Ig heavy chain locus (del A1-GP-G2-G4-E). J Immunol. 1999 Oct. 15; 163(8):4392-8.PMID: 10510380
43. Lefranc M-P L G. The Immunoglobulin Factsbook. London: Academic Press; 2001.
44. Lincoln M R, Ramagopalan S V, Chao M J, Herrera B M, DeLuca G C, Orton S-M M, Dyment D a, Sadovnick a D, Ebers G C. Epistasis among HLA-DRB1, HLA-DQA1, and HLA-DQB1 loci determines multiple sclerosis susceptibility. Proc Natl Acad Sci. 2009; 106(18):7542-7547. PMID: 19380721
45. de Bakker P I W, McVean G, Sabeti P C, Miretti M M, Green T, Marchini J, Ke X, Monsuur A J, Whittaker P, Delgado M, Morrison J, Richardson A, Walsh E C, Gao X, Galver L, Hart J, Hafler D A, Pericak-Vance M, Todd J A, Daly M J, Trowsdale J, Wijmenga C, Vyse T J, Beck S, Murray S S, Carrington M, Gregory S, Deloukas P, Rioux J D. A high-resolution HLA and SNP haplotype map for disease association studies in the extended human MEW. Nat Genet. 2006; 38(10):1166-72. PMID: 16998491
46. Yun J, Adam J, Yerly D, Pichler W J. Human leukocyte antigens (HLA) associated drug hypersensitivity: Consequences of drug binding to HLA. Allergy Eur J Allergy Clin Immunol. 2012; 67(11):1338-1346.PMID: 22943588
47. Amstutz U, Ross C, Castro-Pastrana L, Rieder M, Shear N, Hayden M R, Carleton B C, Consortium C. HLA-A*31:01 and HLA-B*15:02 as genetic markers for carbamazepine hypersensitivity in children. Clin Pharmacol Ther. 2014; 94(1):1-18. PMID: 23588310
48. Hashimoto L L, Walter M A, Cox D W, Ebers G C. Immunoglobulin heavy chain variable region polymorphisms and multiple sclerosis susceptibility. J Neuroimmunol. 1993; 44(1):77-83. PMID:8496340
49. Cho M-L, Chen P P, Seo Y-I, Hwang S-Y, Kim W-U, Min D-J, Park S-H, Cho C-S. Association of homozygous deletion of the Humhv3005 and the VH3-30.3 genes with renal involvement in systemiclupus erythematosus. Lupus. 2003; 12(5):400-5. PMID: 12765304
50. Walter M a, Gibson W T, Ebers G C, Cox D W. Susceptibility to multiple sclerosis is associated with the proximal immunoglobulin heavy chain variable region. J Clin Invest. 1991; 87(4):1266-73. PMID:1672695
51. Cortes A, Brown M A. Promise and pitfalls of the Immunochip. Arthritis Res Ther. 2011; 13(1):101. PMID:21345260
52. Sudmant P H, Rausch T, Gardner E J, Handsaker R E, Abyzov A, Huddleston J, Zhang Y, Ye K, Jun G, Hsi-Yang Fritz M, Konkel M K, Malhotra A, Stutz A M, Shi X, Paolo Casale F, Chen J, Hormozdiari F, Dayama G, Chen K, Malig M, Chaisson M J P, Walter K, Meiers S, Kashin S, Garrison E, Auton A, Lam H Y K, Jasmine Mu X, Alkan C, Antaki D, Bae T, Cerveira E, Chines P, Chong Z, Clarke L, Dal E, Ding L, Emery S, Fan X, Gujral M, Kahveci F, Kidd J M, Kong Y, Lameijer E-W, McCarthy S, Flicek P, Gibbs R A, Marth G, Mason C E, Menelaou A, Muzny D M, Nelson B J, Noor A, Parrish N F, Pendleton M, Quitadamo A, Raeder B, Schadt E E, Romanovitch M, Schlattl A, Sebra R, Shabalin A A, Untergasser A, Walker J A, Wang M, Yu F, Zhang C, Zhang J, Zheng-Bradley X, Zhou W, Zichner T, Sebat J, Batzer M A, McCarroll S A, Mills R E, Gerstein M B, Bashir A, Stegle O, Devine S E, Lee C, Eichler E E, Korbel J O. An integrated map of structural variation in 2,504 human genomes. Nature. 2015; 526(7571):75-81. PMID: 26432246
53. Auton A, Abecasis G R, Altshuler D M, Durbin R M, Bentley D R, Chakravarti A, Clark A G, Donnelly P, Eichler E E, Flicek P, Gabriel S B, Gibbs R A, Green E D, Hurles M E, Knoppers B M, Korbel J O, Lander E S, Lee C, Lehrach H, Mardis E R, Marth G T, McVean G A, Nickerson D A, Schmidt J P, Sherry S T, Wang J, Wilson R K, Boerwinkle E, Doddapaneni H, Han Y, Korchina V, Kovar C, Lee S, Muzny D, Reid J G, Zhu Y, Chang Y, Feng Q, Fang X, Guo X, Jian M, Jiang H, Jin X, Lan T, Li G, Li J, Li Y, Liu S, Liu X, LuY, Ma X, Tang M, Wang B, Wang G, Wu H, Wu R, Xu X, Yin Y, Zhang D, Zhang W, Zhao J, Zhao M, Zheng X, Gupta N, Gharani N, Toji LH, Gerry N P, Resch A M, Barker J, Clarke L, Gil L, Hunt S E, Kelman G, Kulesha E, Leinonen R, McLaren W M, Radhakrishnan R, Roa A, Smirnov D, Smith R E, Streeter I, Thormann A, Toneva I, Vaughan B, Zheng-Bradley X, Grocock R, Humphray S, James T, Kingsbury Z, Sudbrak R, Albrecht M W, Amstislayskiy V S, Borodina T A, Lienhard M, Mertes F, Sultan M, Timmermann B, Yaspo M-L, Fulton L, Fulton R, Ananiev V, Belaia Z, Beloslyudtsev D, Bouk N, Chen C, Church D, Cohen R, Cook C, Garner J, Hefferon T, Kimelman M, Liu C, Lopez J, Meric P, O'Sullivan C, Ostapchuk Y, Phan L, Ponomarov S, Schneider V, Shekhtman E, Sirotkin K, Slotta D, Zhang H, Balasubramaniam S, Burton J, Danecek P, Keane T M, Kolb-Kokocinski A, McCarthy S, Stalker J, Quail M, Davies C J, Gollub J, Webster T, Wong B, Zhan Y, Campbell C L, Kong Y, Marcketta A, Yu F, Antunes L, Bainbridge M, Sabo A, Huang Z, Coin L J M, Fang L, Li Q, Li Z, Lin H, Liu B, Luo R, Shao H, Xie Y, Ye C, Yu C, Zhang F, Zheng H, Zhu H, Alkan C, Dal E, Kahveci F, Garrison E P, Kural D, Lee W P, Fung Leong W, Stromberg M, Ward A N, Wu J, Zhang M, Daly M J, DePristo M A, Handsaker R E, Banks E, Bhatia G, del Angel G, Genovese G, Li H, Kashin S, McCarroll S A, Nemesh J C, Poplin R E, Yoon S C, Lihm J, Makarov V, Gottipati S, Keinan A, Rodriguez-Flores J L, Rausch T, Fritz M H, Stütz A M, Beal K, Datta A, Herrero J, Ritchie G R S, Zerbino D, Sabeti P C, Shlyakhter I, Schaffner S F, Vitti J, Cooper D N, Ball E V., Stenson P D, Barnes B, Bauer M, Keira Cheetham R, Cox A, Eberle M, Kahn S, Murray L, Peden J, Shaw R, Kenny E E, Batzer M A, Konkel M K, Walker J A, MacArthur D G, Lek M, Herwig R, Ding L, Koboldt D C, Larson D, Ye K, Gravel S, Swaroop A, Chew E, Lappalainen T, Erlich Y, Gymrek M, Frederick Willems T, Simpson J T, Shriver M D, Rosenfeld J A, Bustamante C D, Montgomery S B, De La Vega F M, Byrnes J K, Carroll A W, DeGorter M K, Lacroute P, Maples B K, Martin A R, Moreno-Estrada A, Shringarpure S S, Zakharia F, Halperin E, Baran Y, Cerveira E, Hwang J, Malhotra A, Plewczynski D, Radew K, Romanovitch M, Zhang C, Hyland F C L, Craig D W, Christoforides A, Homer N, Izatt T, Kurdoglu A A, Sinari S A, Squire K, Xiao C, Sebat J, Antaki D, Gujral M, Noor A, Ye K, Burchard E G, Hernandez R D, Gignoux C R, Haussler D, Katzman S J, James Kent W, Howie B, Ruiz-Linares A, Dermitzakis E T, Devine S E, Min Kang H, Kidd J M, Blackwell T, Caron S, Chen W, Emery S, Fritsche L, Fuchsberger C, Jun G, Li B, Lyons R, Scheller C, Sidore C, Song S, Sliwerska E, Taliun D, n A Welch R, Kate Wing M, Zhan X, Awadalla P, Hodgkinson A, Li Y, Shi X, Quitadamo A, Lunter G, Marchini J L, Myers S, Churchhouse C, Delaneau O, Gupta-Hinch A, Kretzschmar W, Iqbal Z, Mathiesonl, Menelaou A, Rimmer A, Xifara D K, Oleksyk T K, Fu Y, Liu X, Xiong M, Jorde L, Witherspoon D, Xing J, Browning B L, Browning S R, Hormozdiari F, Sudmant P H, Khurana E, Tyler-Smith C, Albers C A, Ayub Q, Chen Y, Colonna V, Jostins L, Walter K, Xue Y, Gerstein M B, Abyzov A, Balasubramanian S, Chen J, Clarke D, Fu Y, Harmanci A O, Jin M, Lee D, Liu J, Jasmine Mu X, Zhang J, Zhang Y, Hartl C, Shakir K, Degenhardt J, Meiers S, Raeder B, Paolo Casale F, Stegle O, Lameijer E-W, Hall I, Bafna V, Michaelson J, Gardner E J, Mills R E, Dayama G, Chen K, Fan X, Chong Z, Chen T, Chaisson M J, Huddleston J, Malig M, Nelson B J, Parrish N F, Blackburne B, Lindsay S J, Ning Z, Zhang Y, Lam H, Sisu C, Challis D, Evani U S, Lu J, Nagaswamy U, Yu J, Li W, Habegger L, Yu H, Cunningham F, Dunham I, Lage K, Berg Jespersen J, Horn H, Kim D, Desalle R, Narechania A, Wilson Sayres M A, Mendez F L, David Poznik G, Underhill P A, Coin L, Mittelman D, Banerjee R, Cerezo M, Fitzgerald T W, Louzada S, Massaia A, Ritchie G R, Yang F, Kalra D, Hale W, Dan X, Barnes K C, Beiswanger C, Cai H, Cao H, Henn B, Jones D, Kaye J S, Kent A, Kerasidou A, Mathias R, Ossorio P N, Parker M, Rotimi C N, Royal C D, Sandoval K, Su Y, Tian Z, Tishkoff S, Via M, Wang Y, Yang H, Yang L, Zhu J, Bodmer W, Bedoya G, Cai Z, Gao Y, Chu J, Peltonen L, Garcia-Montero A, Orfao A, Dutil J, Martinez-Cruzado J C, Mathias R A, Hennis A, Watson H, McKenzie C, Qadri F, LaRocque R, Deng X, Asogun D, Folarin O, Happi C, Omoniwa O, Stremlau M, Tariyal R, Jallow M, Sisay Joof F, Corrah T, Rockett K, Kwiatkowski D, Kooner J, Tinh Hié'n T, Dunstan S J, Thuy Hang N, Fonnie R, Garry R, Kanneh L, Moses L, Schieffelin J, Grant D S, Gallo C, Poletti G, Saleheen D, Rasheed A, Brooks L D, Felsenfeld A L, McEwen J E, Vaydylevich Y, Duncanson A, Dunn M, Schloss J A. A global reference for human genetic variation. Nature. 2015; 526(7571):68-74. PMID: 26432245
54. Luo S, Yu J A, Song Y S. Estimating Copy Number and Allelic Variation at the Immunoglobulin Heavy Chain Locus Using Short Reads. PLoS Comput Biol. 2016; 12(9):1-21. PMID: 27632220
55. Luo S, Yu J A, Li H, Song Y S. Worldwide genetic variation of the IGHV and TRBV immune receptor gene families in humans. 2017; 1-18. doi:http://dx.doi.org/10.1101/155440.
56. Ralph D K, Matsen F A. Consistency of VDJ Rearrangement and Substitution Parameters Enables Accurate B Cell Receptor Sequence Annotation. PLoS Comput Biol. 2016; 12(1):1-25. PMID: 26751373
57. Kirik U, Greiff L, Levander F, Ohlin M. Parallel antibody germline gene and haplotype analyses support the validity of immunoglobulin germline gene inference and discovery. Mol Immunol. 2017; 87:12-22.PMID: 28388445
58. Feeney A J, Atkinson M J, Cowan M J, Escuro G, Lugo G. A defective V kappa A2 allele in Navajos which may play a role in increased susceptibility to haemophilus influenzae type b disease. J Clin Invest. 1996; PMID: 8636407
59. Watson C T, Steinberg K M, Graves T, Warren R L, Malig M, Schein J, Wilson R K, Holt R, Eichler E E, Breden F. Sequencing of the human IG light chain loci from a hydatidiform mole BAC library reveals locus-specific signatures of genetic diversity. Genes Immun. 2014; PMCID: PMC4304971
60. Chaisson M J, Tesler G. Mapping single molecule sequencing reads using basic local alignment with successive refinement (BLASR): application and theory. BMC Bioinformatics. 2012; 13(1):238. PMID:22988817
61. Chin C-S, Alexander D H, Marks P, Klammer A A, Drake J, Heiner C, Clum A, Copeland A, Huddleston J, Eichler E E, Turner S W, Korlach J. Nonhybrid, finished microbial genome assemblies from long-read SMRT sequencing data. Nat Methods. 2013 June; 10(6):563-9. PMID: 23644548
62. Patterson M, Marschall T, Pisanti N, van Iersel L, Stougie L, Klau G W, Schonhuth A. Weighted Haplotype Assembly for Future-Generation Sequencing Reads. J Comput Biol. 2015; 22(6):498-509.PMID: 25658651
63. Rodriguez O. https://bitbucket.org/oscarlr/mspac.
64. Ellebedy A H, Jackson K J L, Kissick H T, Nakaya H I, Davis C W, Roskin K M, Mcelroy A K, Oshansky C M, Elbein R, Thomas S, Lyon G M, Spiropoulou C F, Mehta A K, Thomas P G, Boyd S D, Ahmed R. Defining antigen-specific plasmablast and memory B cell subsets in human blood after viral infection or vaccination. 2016; 17(10). PMCID: PMC5054979
65. Looney T J, Lee J, Roskin K M, Hoh R A, King J, Glanville J, Liu Y, Pham T D, Dekker C L, Davis M M. Human B-cell isotype switching origins of IgE. J Allergy Clin Immunol. 2016; 137(2):579-586.e7. PMID:26309181
66. Gupta N T, Heiden J A Vander, Uduman M, Gadala-maria D, Yaari G, Kleinstein H. Change-O: a toolkit for analyzing large-scale B cell immunoglobulin repertoire sequencing data. 2015; 31:3356-3358.PMCID: PMC4793929
67. Heiden J A Vander, Yaari G, Uduman M, Stern J N H, Connor K C O, Hafler D A, Vigneault F, Kleinstein S H. pRESTO: a toolkit for processing high-throughput sequencing raw reads of lymphocyte receptor repertoires. 2014; 30(13):1930-1932. PMCID: PMC4071206
68. Kirik U, Persson H, Levander F, Greiff L, Ohlin M. Antibody Heavy Chain Variable Domains of Different Germline Gene Origins Diversify through Different Paths. 2017; 8:1-21. PMCID:PMC5694033
69. Silveira J, Armanhi L, Soares R, Souza C De, Araújo L M De. Multiplex amplicon sequencing for microbeidentification in community-based culture collections. Nat Publ Gr. 2016; (July):1-9. PMCID:PMC4941570
70. Qiao W, Yang Y, Sebra R, Mendiratta G, Gaedigk A. Long-read single-molecule real-time (SMRT) fullgene sequencing of cytochrome P450-2D6 (CYP2D6). Hum Mutat. 2016; 37(3):315-323. PMCID:PMC4752389
71. Wagner J, Coupland P, Browne H P, Lawley T D, Francis S C, Parkhill J. Evaluation of PacBiosequencing for full-length bacterial 16S rRNA gene classification. BMC Microbiol. 2016; 1-17. PMCID:PMC5109829
72. Phillips C, Fondevila M, Vallone P M, Carla S, Freire-aradas A, Butler J M, Victoria M, Carracedo A. Characterization of U.S. population samples using a 34-plex ancestry informative SNP multiplex. Forensic Sci Int Genet Suppl Ser. 2011; 3(1):e182-e183.
73. Laurie C C, Doheny K F, Mirel D B, Pugh E W, Laura J, Bhangale T, Boehm F, Caporaso N E, Cornelis M C, Edenberg H J, Gabriel S B, Harris E L, Hu F B, Jacobs K, Kraft P, Landi M T, Lumley T, Manolio T A, Mchugh C, Painter I, Paschall J, Rice J P, Rice K M, Zheng X, Weir B S, GENEVA Investigators. Quality control and quality assurance in genotypic data for genome-wide association studies. Genet Epidemiol. 2011; 34(6):591-602. PMCID: PMC3061487
74. Peters B A, Kermani B G, Sparks A B, Alferov O, Hong P, Alexeev A, Jiang Y, Dahl F, Tang Y T, Haas J, Robasky K, Zaranek A W, Lee J-H, Ball M P, Peterson J E, Perazich H, Yeung G, Liu J, Chen L, Kennemer M I, Pothuraju K, Konvicka K, Tsoupko-Sitnikov M, Pant K P, Ebert J C, Nilsen G B, Baccash J, Halpern A L, Church G M, Drmanac R. Accurate whole-genome sequencing and haplotyping from 10 to20 human cells. Nature. 2012 July; 487(7406):190-5. PMID: 22785314
75. Kohsaka H, Carson D A, Rassenti L Z, Ollier WER, Chen P P, Kipps T J, Miyasaka N. The human immunoglobulin VH gene repertoire is genetically controlled and unaltered by chronic autoimmune stimulation. J Clin Invest. 1996; 98(12):2794-2800. PMID: 8981926
76. Feeney A J. Genetic and epigenetic control of V gene rearrangement frequency. Adv Exp Med Biol. 2009; 650:73-81. PMID: 19731802
77. Sharon E, Sibener L V, Battle A, Fraser H B, Garcia K C, Pritchard J K. Genetic variation in MEW proteins is associated with T cell receptor expression biases. Nat Genet. 2016; 48(9):995-1002. PMID: 27479906
78. Avnir Y, Tallarico A S, Zhu Q, Bennett A S, Connelly G, Sheehan J, Sui J, Fahmy A, Huang C, Cadwell G, Bankston L a, McGuire A T, Stamatatos L, Wagner G, Liddington R C, Marasco W a. Molecular signatures of hemagglutinin stem-directed heterosubtypic human neutralizing antibodies against influenza A viruses. PLoS Pathog. 2014; PMCID: PMC4006906
79. Lerner R a. Rare antibodies from combinatorial libraries suggests an S.O.S. component of the human immunological repertoire. Mol Biosyst. 2011 April; 7(4):1004-12. PMID: 21298133
80. Hwang K K, Trama A M, Kozink D M, Chen X, Wiehe K, Cooper A J, Xia S M, Wang M, Marshall D J, Whitesides J, Alam M, Tomaras G D, Allen S L, Rai K R, McKeating J, Catera R, Yan X J, Chu C C, Kelsoe G, Liao H X, Chiorazzi N, Haynes B F. IGHV1-69 B cell chronic lymphocytic leukemia antibodies crossreact with HIV-1 and hepatitis C virus antigens as well as intestinal commensal bacteria. PLoS One. 2014; 9(3):e90725. PMID: 24614505
81. Vencovsky J, Zdarsky E, Moyes S P, Hajeer A, Ruzickova S, Cimburek Z, Ollier W E, Maini R N, Mageed R A. Polymorphism in the immunoglobulin VH gene V1-69 affects susceptibility to rheumatoid arthritis in subjects lacking the HLA-DRB1 shared epitope. Rheumatology. 2002; 41:401-410.
82. Pos W, Luken B M, Hovinga J A K, Turenhout E A M, Scheiflinger F. VH1-69 germline encoded antibodies directed towards ADAMTS13 in patients with acquired thrombotic thrombocytopenic purpura. 2009; 7(3):421-428. PMID: 19054323
83. Shabalin A A. Matrix eQTL: Ultra fast eQTL analysis via large matrix operations. Bioinformatics. 2012; 28(10):1353-1358. PMID: 22492648
84. Purcell S, Neale B, Todd-Brown K, Thomas L, Ferreira M a R, Bender D, Maller J, Sklar P, de Bakker P l W, Daly M J, Sham P C. PLINK: a tool set for whole-genome association and population-based linkage analyses. Am J Hum Genet. 2007 September; 81(3):559-75. PMID: 17701901
85. Stegle O, Parts L, Piipari M, Winn J, Durbin R. Using probabilistic estimation of expression residuals (PEER) to obtain increased power and interpretability of gene expression analyses. Nat Protoc. 2012; 7(3):500-7. PMID: 22343431
86. Benjamini Y, Hochberg Y. Controlling the False Discovery Rate: A Practical and Powerful Approach to Multiple Testing. J R Stat Soc Ser B. 1995; 57(1):289-300.
87. Gibson K L, Wu Y, Barnett Y, Duggan O, Vaughan R, Kondeatis E, Nilsson B, Wikby A, Kipling D, Dunnwalters D K. B-cell diversity decreases in old age and is correlated with poor health status. 2009; 8(1):18-25. PMCID: PMC2667647

### Example 15

Specific Aims

Genes at the immunoglobulin heavy chain (IGH) and light chain loci recombine to form expressed antibodies (Ab). Despite the importance of the Ab response in human immunity, little is known about the role of IGH genetics in Ab repertoire diversity. This stems from the fact that IGH has been largely ignored by most human immunogenetic studies, due in part to extreme diversity at the genomic and population level. However, recent advances allow us to address this fundamentally important question. First, a combination of new sequencing technologies and bioinformatics approaches facilitates development of tools for high-throughput IGH haplotype characterization. Second, high resolution descriptions of naïve and antigen-stimulated Ab responses are possible via repertoire sequencing. Using a well-characterized, multi-ethnic seasonal influenza vaccinee cohort, this example will examine the contribution of IGH polymorphisms to inter-individual variability in the expressed Ab repertoire and associated neutralizing Ab response. Specifically, this will result in a comprehensive catalogue of IGH haplotype variation, new tools for locus-wide IGH genotyping, and an understanding of how IGH germline differences impact variability in the naïve and antigen-stimulated Ab repertoire in the context of seasonal influenza vaccination. This work will expand our understanding of the contribution of IGH polymorphism to the expressed Ab repertoire in human disease, and establish desperately needed genomic resources for the IGH locus, forming a foundation for integrating Ab profiling in the burgeoning field of personalized medicine.

Aim 1. A Comprehensive Human IGH Haplotype Map/Variant Catalogue and Construction of New High Throughput Assays for IGH Genotyping in Clinical Populations.

We will study a diverse population (African, Asian, European, and Hispanic), including: (i) a panel of nine fosmid libraries, and (ii) whole-genome long-read sequence (WGS) data generated using Pacific Biosciences (PacBio) SMRT sequencing from four trios (mother, father, child). These haplotype maps will be expanded by incorporating targeted sequencing and single nucleotide polymorphism (SNP) data from a larger panel of diverse human population samples. Genetic variation from these sources will be integrated into a population reference graph (PRG) that will dramatically expand upon all known forms of IGH germline variation, including copy number variants (CNVs), structural variants, and SNPs/Indels. Leveraging the PRG, we will develop a comprehensive IGH DNA sequence capture assay and accompanying informatics pipeline. This will include genotype calls for locus-wide CNVs and SNPs; IGH variable (V), diversity (D), joining (J), and constant (C) genes and alleles; and regulatory region variation.

Aim 2. Phenotypic Characterization of Key Signatures of the Dynamic Ab Repertoire in a Multi-Ethnic Cohort Before and After Seasonal Influenza Vaccination.

We will use next-generation sequencing (NGS) to analyze circulating IgM and IgG Ab repertoires from 183 healthy volunteers of diverse ethnicity, a subset of which (n=138) participated in a seasonal influenza study and provided peripheral blood samples at pre-vaccination, 7 days post-vaccination (plasmablast peak), and 30-56 days post-vaccination (memory B cell pool). To advance current methods of sequencing only unpaired IGHV genes, we will develop a new drop-seq platform that will allow high-throughput in situ assembly of cognate heavy and light chain V gene (VH-VL) pairs from single B cells, linked to yeast display. Expression levels of all IGH V, D, and J germline genes, and other naïve and memory repertoire features will be obtained pre- and post-vaccination. Our studies will also focus on broadly neutralizing Abs (BnAbs) against the highly conserved hemagglutinin (HA) stem domain that forms the basis of current universal influenza vaccine candidates. Cognate VH-VL pairs assembled from drop-seq runs will be used to produce barcoded libraries of single-chain antibodies (scFvs) that will be FACS-sorted for simultaneously binding multiple fluorochrome tagged influenza HA trimers of non-contemporaneous influenza A and B strains. Each BnAb derived from a single B cell will form the functional nodes to assess clonal diversity and expansion. This will facilitate construction of a database of anti-influenza BnAb molecular signatures to be interrogated for genetic linkages in Aim 3.

Aim 3. Characterize IGH Variants that Impact Signatures in Naïve and Ag-Stimulated Expressed Antibody Repertoires Pre- and Post-Vaccination for Seasonal Influenza.

The role of IGH germline variants in Ab expression and function have yet to be defined. By combining the genotypes in Aim 1 with repertoire data collected in Aim 2, we will conduct a genetic association analysis to characterize functional IGH variants associated with Ab signatures linked to vaccination. Given that the naïve repertoire serves as the baseline for initial Ab-mediated responses, we will first establish a database of functional IGH variants that robustly associate with heritable features of IgM Ab repertoires characterized in this cohort: (1) IGHV-, D-, and J-gene usage frequencies; (2) IGHV, D, and J allele-specific usage; (3) V-D and D-J recombination frequencies; and (4) cognate heavy and light chain V gene pairing bias. We will also explore genetic associations with features in the memory IgG Ab repertoire: specifically, IGH gene/allele/recombination frequencies, as well as signatures associated with B cell expansion, clonal diversity, and somatic hypermutation. Finally, we will test for associations between IGH variants and circulating Ab titers pre- and post-vaccination, particularly focusing on BnAbs of interest in this cohort.

Significance:

The immunoglobulin heavy (IGH) and light chain (IGK, IGL) gene regions, along with the human leukocyte antigen (HLA) and T cell receptor (TCR) loci, code for the three most critical structural components of the adaptive immune system 1. Whereas hundreds of risk alleles for common human diseases map to the HLA locus 2, to date few diseases have been consistently linked to the IG loci 3. This is surprising given that IG genes encode antibodies (Abs), which are key players in autoimmune and infectious disease 4-7.

The IGH locus consists of approximately 54 V, 23 D, 6 J, and 9 C “functional” genes that contribute to the formation of expressed IG heavy chains. Even based on the limited surveys conducted to date, >250 functional IGH alleles are known to occur 8. The locus is also highly enriched for large copy number variants (CNVs), including insertions, deletions, and duplications of functional genes 9-15, and these show considerable variation with evidence of selection among human populations 10,15. This extreme amount of allelic and structural variability has made IGH nearly inaccessible to high-throughput assays, and as a result it has been largely ignored by genome-wide studies3. This has severely impeded our understanding of the contribution of IGH polymorphism to disease risk, infection and response to vaccines and therapeutics. Even more fundamentally, in contrast to most genes in the genome, which have been included in expression quantitative trait loci (eQTL) analyses, we know very little about genetic factors dictating the regulation of the human Ab response.

Although the role of IG germline variants in Ab function was of great interest to the field in earlier decades, its importance was later superseded by a focus on non-genetic factors (e.g., somatic hypermutation, SHM). However, evidence continues to accumulate in support of IGH genetic variation being critically important to the human B cell-driven immune response. First, several studies have shown that particular signatures in expressed naïve and memory Ab repertoires of monozygotic twins are heritable 16-18. This is consistent with limited observations implicating IG gene CNVs and regulatory polymorphisms in inter-individual Ab repertoire variability 9,13,14,19. Second, it is now clear that the Ab response in disease is not simply a random process, as indicated by consistent biases in Ab germline gene usage in various infectious and autoimmune diseases 6,7,20,21, and many cases in which specific IG coding variants lead to differences in Ab function and binding 6,20-25. Together these findings strongly motivate work seeking to comprehensively characterize links between locus-wide IGH germline polymorphism at the population level, variability in the expressed baseline naïve and memory Ab repertoires, and the functional Ab response associated with clinical phenotypes.

The broadly neutralizing Ab (BnAb) response to influenza is characterized by several key features that offer a unique opportunity to assess specific functional impacts of IGH genetic diversity in the context of disease. First, the anti-influenza response varies considerably among individuals in the human population. In addition, we and others have shown that BnAbs directed to the stem of hemagglutinin (HA) (sBnAbs) are strongly biased in their usage of a restricted set of IGH genes, most prominently IGHV1-6920,24,26,27. Notably, a key germline-encoded amino acid (F54) in complementarity determining region, H2 (CDR-H2) loop of IGHV1-69 can facilitate BnAb antigen binding with limited SHM27, and we have shown that the frequency and copy number of IGHV1-69 alleles varies in the population, and directly associates with the IGHV1-69sBnAb response following influenza vaccination 28. In fact, up to 41% of individuals, depending on ethnicity, lack critical IGHV1-69 germline alleles in their genomes, indicating that vaccines aiming to elicit specific IGHV1-69sBnAbs may be less effective in some members of the population 28. This may be compensated by biased use of several other IGH genes, which have now been implicated in the sBnAb vaccine response—albeit at lower frequencies—but have not been studied at the genetic level (FIG. 28) 20,26,29-41. This also more generally demonstrates that, in part due to IG genetics, not all individuals are poised to mount the same Ab-driven response, highlighting the ability for the combined use of Ab genetic and repertoire signatures in partitioning patient populations for personalized care. Indeed, such models have already proven effective for many other genes for which information such as eQTLs is available 42. However, IGH polymorphisms investigated thus far have been limited to a miniscule fraction of the 1000's of IGH variants known15,43. A thorough investigation of IGH locus-wide variation will be necessary to clarify the role

In addition to creating actionable results on influenza vaccination, this work will provide desperately needed insights into our basic understanding of Ab function in disease through the advancement of IGH genomic resources and complete characterization of associations between IGH polymorphisms and features in expressed Ab repertoires at the population level. In addition, the haplotype map resource, genotyping tools and database of functional variants resulting from this example will allow both research and clinical laboratories to incorporate IGH genotyping into their workflows, and provide a basic framework for improving the interpretation of Ab repertoire data and the B cell response in human phenotypes.

In the past decade, high-throughput genomics assays, including SNP microarrays, exome-sequencing, and whole-genome sequencing (WGS), have become ubiquitous. However, complex, repetitive regions of the genome rich in structural variation (SV), such as the IGH locus, continue to present considerable challenges for these technologies. Despite the fact that complex regions often harbor functional variants linked to disease when targeted studies are done 44,45, until genomic resources and tools are readily available, most investigators simply exclude them. The result in IGH is that our knowledge of specific genomic factors involved in Ab repertoire development and variability remains limited to data from inbred mice 46-48, even though such questions would have much greater relevance to human health if addressed in outbred human populations. However, the current genome references so poorly represent population diversity at the human IGH locus that such questions are difficult to explore in detail. Without wishing to be bound by theory, an innovative two-step approach to resolve these problems can include: (i) comprehensively catalogue allelic and structural variation in the IGH locus across a diverse set of humans, (ii) leverage this resource for the design of custom methods for sequencing and analyzing IGH haplotypes in any sample. This will yield a comprehensive haplotype and germline variant resource for IGH, including SNPs and CNVs, and establish a crucial foundation for researchers generating genomics datasets.

Without wishing to be bound by theory, IGH CNVs, and polymorphisms within coding and regulatory regions will strongly influence the Ab repertoire, with a major role in determining an individual's immune response. The use of IGH genomics with Ab repertoire screening will be the first to directly test for connections between locus-wide IGH polymorphisms and repertoire-wide Ab signatures, as a means to better define the functional B cell response. Further advancing the field, we will use a new pipeline to identify key functional sBnAb signatures in the expressed Ab repertoire, facilitated by the development of a single B cell drop-seq platform for next-generation sequence (NGS) analysis of cognate heavy and light chain V gene (VH-VL) pairing, coupled with Ab-yeast display to recover and interrogate anti-influenza sBnAb clones.

Approach:

This Example integrates comprehensive IGH genomic and phenotypic profiling data to define the role of IGH germline variation in the functional Ab response. In Aim 1, we will build a comprehensive database of IGH haplotype variation using: (i) a panel of nine fosmid libraries, and (ii) long-read WGS data generated using Pacific Biosciences (PacBio) SMRT sequencing from four human parent-child trios of diverse ethnic origin (African, Asian, European, and Hispanic). Together, this will provide ˜34 new IGH haplotypes from 17 unrelated individuals, plus additional data from four offspring. These new haplotypes will more accurately reflect the complex structure of IGH and reveal sequences missing from current references. In addition, we will assess IGHV coding diversity in a larger panel of diverse individuals using targeted approaches and mine the 1000 Genomes Project (1KGP) 49,50 database for new SNP variation in IGH. Once curated, genetic variation will be integrated into a population reference graph (PRG) that will dramatically expand upon all known forms of IGH germline variation. Leveraging this resource, we will design a custom capture assay and bioinformatics toolkit for comprehensive genotyping of CNVs and SNPs in IGH. After validating these assays, we will apply them to generate IGH genotypes in two study cohorts: (i) 138 healthy volunteers that participated in a seasonal influenza vaccine study (FIG. 29), and (ii) 45 anonymous healthy blood bank donors. These data will permit the first locus-wide IGH population genetics study and serve as a foundation for eQTL analyses in Aim 3.

In Aim 2, we will use NGS to analyze circulating IgM and IgG Ab repertoires from seasonal influenza study volunteers (cohort 1) who provided peripheral blood samples at pre-vaccination, 7 days post-vaccination (plasmablast peak), and 30 days post-vaccination (memory B cell pool); we will also generate IgM and IgG repertoires from a single blood draw for samples in cohort 2. We will develop a new drop-seq platform that will allow high-throughput in situ assembly of cognate VH-VL pairs from single B cells, and will be linked to yeast display. Expression levels of all IGH V, D, and J germline genes during the vaccine response will be obtained. Our studies will also focus on sBnAbs against the highly conserved HA stem domain (the basis of current universal influenza vaccine candidates). VH-VL pairs assembled from drop-seq runs will be used to produce single-chain antibody (scFvs) libraries that will be FACS-sorted for simultaneously binding multiple fluorochrome tagged influenza HA trimers of non-contemporaneous influenza A and B strains. Each BnAb derived from a single B cell will form the functional nodes to assess clonal diversity and expansion. This will facilitate construction of an anti-influenza sBnAb molecular signatures database to be interrogated in Aim 3.

Finally, in Aim 3 we will explore relationships between IGH polymorphism and the functional antibody response before and after seasonal influenza vaccination. We have previously shown 28 that variability in the Ab repertoire is linked to IGH genotype and can inform the functional B cell response, with differences between human populations. A priori, connections between IGH genotype and expressed Ab repertoires can result given that the IGH germline is the precursor from which Ab diversity is generated; however, this basic question has not been comprehensively investigated. We will therefore perform an eQTL association analysis of IGH sequence polymorphism and signatures of the naïve and memory repertoires in healthy adults at baseline (cohorts 1 and 2) and at two time points after seasonal influenza vaccination (cohort 1). Associations between IGH variants and circulating titres of Abs of interest will also be investigated. This will be the first large-scale population study to investigate the role of IGH polymorphism in expressed Ab repertoire variation, and will result in a catalogue of functional genomic variants, which can inform the interpretation of the Ab-mediated response in a range of biomedical contexts.

Data: Describing IGH Structural Variation Using Large-Insert Clone Libraries.

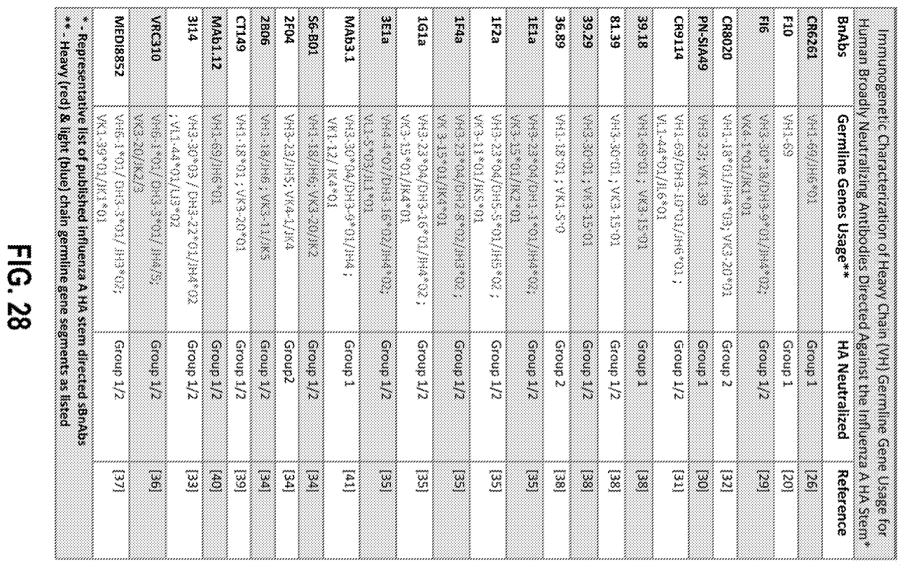

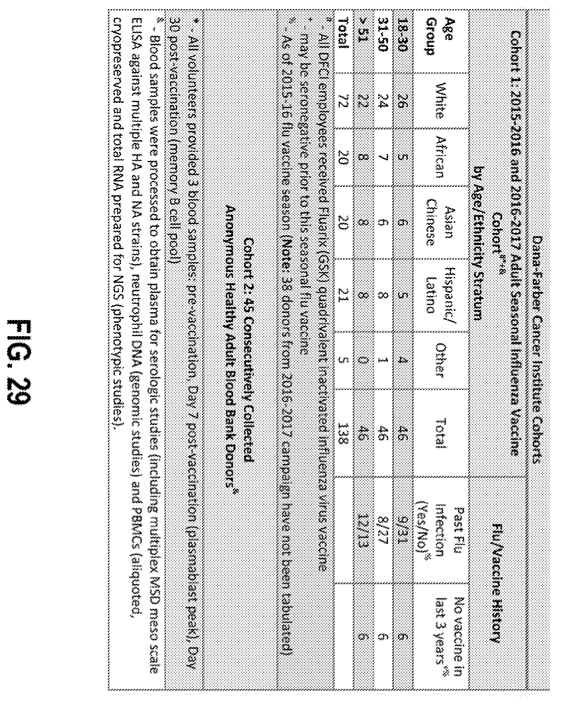

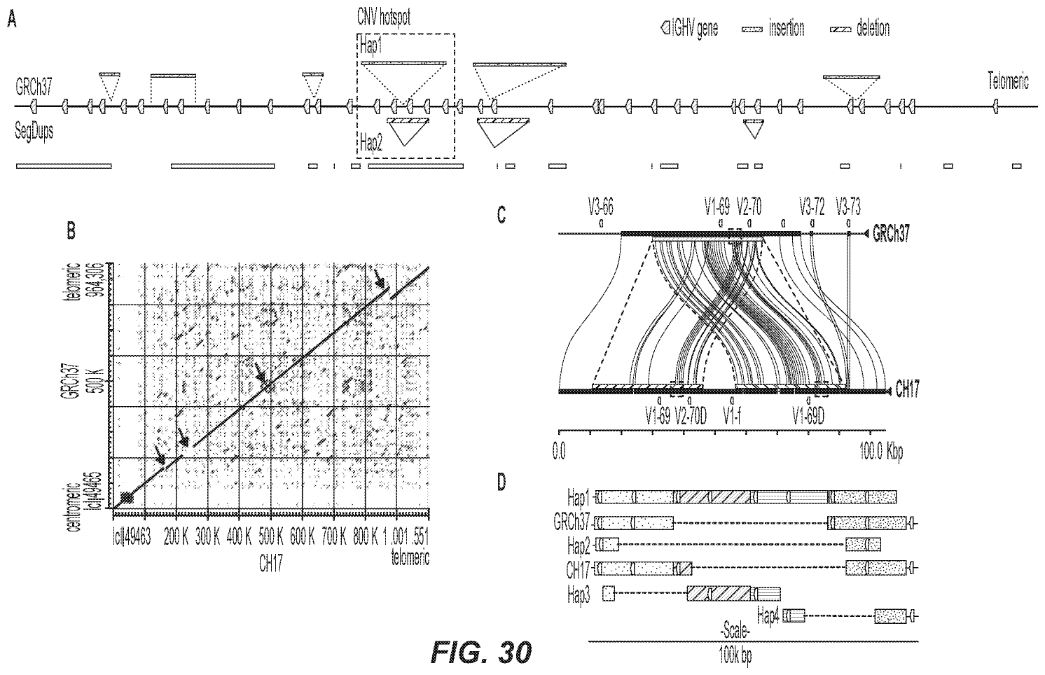

We have undertaken the largest resequencing effort in IGH to date 15, utilizing the CH17 haploid hydatidiform BAC library and fosmid libraries from three human populations (African, Asian, and European) (FIG. 30). Based on CH17, we characterized the first complete sequence of IGH V, D, and J regions from a single chromosome. This newly constructed IGH haplotype differed from GRCh37 in gene copy number of 10 IGHV genes, and allelic differences were observed at 18/40 functional genes; strikingly, CH17 included >100 kb of new sequence (FIG. 30). This demonstrated that even between just two chromosomes there may be major IGH functional differences. Additionally, we observed >2,800 SNPs between these two IGH references, a density ˜3- to 6-fold higher than that observed at other immune loci. From targeted fosmid assemblies, we characterized seven additional IGH CNV regions (FIG. 30) and an additional >120 kb of new inserted sequence; together with the CH17 assembly, increasing the length of available reference sequence in IGHV by >20%15.

Long-Read Haplotype Assembly of NA12878 and Trio Sequencing from the 1kG and GIAB Consortiums.

Long-reads offer improved read-backed phasing compared to short-read approaches, as well as the ability to exhaustively resolve complex SVs 51-53. We recently performed the first de novo assembly of a diploid genome using PacBio long reads (on the NA12878 cell-line), with an automated process approaching reference quality. Haplotype phasing via a combination of short- and long-read approaches, produced long haplotype blocks and resolved unphased variants from trio-based approaches. We were able to assign 12,758 Tandem repeats and SVs to their maternal or paternal haplotype, including events in IGH. Using multiple technology platforms including PacBio as part of the 1KGP Structural Variation and Genome in a Bottle (GIAB) consortia, we are able to achieve reference-based phasing with N50s (the length for which the collection of all contigs of that length or longer contains at least half the genome) in the tens of Mb54 and de novo phasing with N50s in the Mb.

Fosmid Haplotype Assembly with PacBio Longreads.

We have tested the use of a new fosmidpooling/PacBio sequencing approach (Aim 1.1) on a single non-overlapping fosmid path in IGH of 18 clones in an African individual (FIG. 31). This analysis has already resulted in the first genomic characterization of a 9.7 Kb deletion (FIG. 31) known to impact IGHD-J gene recombination 13,55.

Testing a high-throughput platform for IGH genotyping. We conducted an IGH capture-sequencing experiment on two human samples. Nimblegen SeqCap probes were designed across IGH using our previously published haplotype data corresponding to ˜1.4 Mb of unique sequence targets. With this design we tested two capture protocols for sequencing with the Illumina MiSeq and PacBio. Reads were mapped to both the current reference assembly (GRCh37) and our alternate CH17 IGH haplotype (GRCh38)15, allowing >5,000 SNP calls, and the identification of known duplications and deletions: the IGHV 1-69 region is shown in FIG. 31. This analysis highlights the power of pairing two sequencing platforms and multiple references for the identification of IGH CNVs. Clear signatures were observed in the MiSeq read depth profiles (FIG. 31), and PacBio long reads allowed for the disambiguation of duplicated segments (FIG. 31). Our group and others have shown that integrating PacBio continuous long reads (CLRs) with deep coverage short-read data is the most information-rich sequencing approach, and can yield assembly accuracies >Q6056-58.

See, for example, https://www.pacb.com/wp-content/uploads/Procedure-Checklist-%E2%80%93-Multiplex-Genomic-DNA-Target-Capture-Using-SeqCap-EZ-Libraries.pdf, which is incorporated by reference herein in its entirety.

IGHV 1-69 Allelic and Copy Number Variation have Functional Consequences on the Ab Repertoire Associated with Influenza Vaccination.

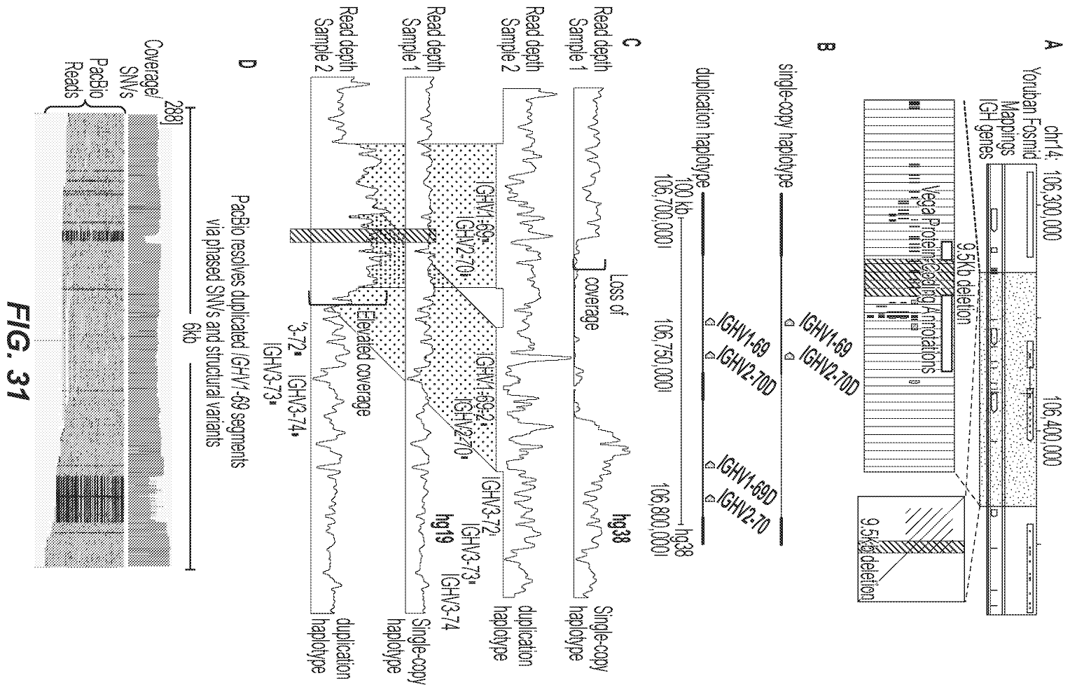

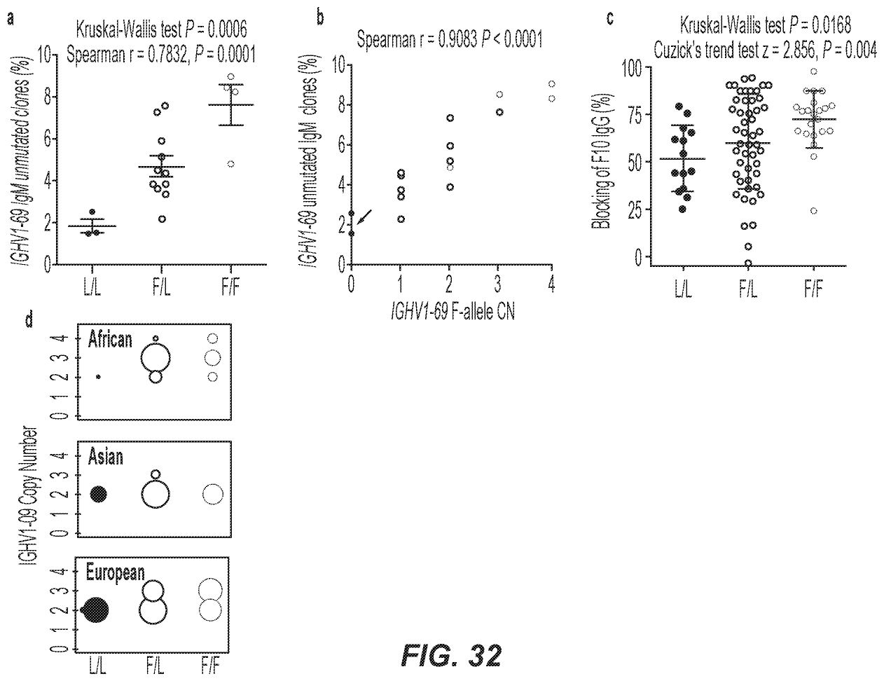

There is now mounting data challenging the notion that the development of the Ab repertoire is simply a stochastic process, and that genetically determined baseline differences in the Ab repertoire can set the stage for variation in disease-related responses. We have begun to explore this idea in detail at the IGHV 1-69 locus 28. This region is complex, characterized by both CNV (FIG. 31) and allelic variation, with 14 alleles residing on haplotypes that can carry either one or two haploid gene copies. IGHV 1-69 alleles are subdivided into two groups defined by either CDR-H2 F54 (51p1 alleles) or CDR-H2 L54 (hv1263 alleles). The CDRH2 F54 substitution has fundamental function in influenza HA stembinding 20,27. Depending on their genotype at IGH, individuals can carry between zero and four copies of CDRH2 F54 alleles. Using qPCR, we genotyped the IGHV 1-69 L/F allele and gene copy number in a cohort of 85 H5N1 vaccinees, including 18 individuals with accompanying Ab repertoire data28. We found robust connections between IGHV1-69 SNPs, CNVs, and repertoire gene usage in both the unmutated IgM (naïve) (FIG. 32) and IgG memory repertoire. Importantly, when looking at the entire cohort of 85, these genotype effects extended to levels of circulating anti-influenza sBnAbs; individuals carrying only CDR-H2 L54 had lower levels of IGHV 1-69 sBnAbs (FIG. 32). Using an extended cohort, we found that the frequency of CDR-H2 L54 alleles and IGHV 1-69 CNV varied considerably across populations, indicating strong population-specific haplotype structure (FIG. 32), and the number of individuals lacking germline precursors of IGHV 1-69 sBnAbs was much higher in some populations. Interestingly, individuals in our cohort with no germline copies of CDR-H2 F54 alleles had a higher ratio of IGHV 1-69 clones in the IgG memory repertoire compared to IgM, and these IgG clones had higher levels of SHM at key IGHV1-69 sBnAb signature sites 28. Together, this work demonstrates clear links between IGH genotype, repertoire, and the functional Ab response, and that genotype information can be useful for providing a more detailed understanding of the Ab response and inform vaccine design. Intriguingly, we also found surprising connections between IGHV 1-69 polymorphism and repertoire usage of genes over 200 Kb away, including IGHV3-30/33rn and IGHV3-23, which exhibited contrasting genotype-associated usage patterns from IGHV 1-69 in both IgM and IgG subsets (FIG. 33). Notably, both IGHV3-30 and IGHV3-23 are known to be highly polymorphic and reside within CNV rich regions of IGH 15,59, and both show biased usage in influenza A sBnAbs (FIG. 28), including our recently published report of biased use of IGHV3-30 in anti-influenza sBnAbs that neutralize both group 1 and 2 strains 33.

Research Plan

Aim 1. Resolving a Comprehensive Human IGH Haplotype Map and Constructing New High-Throughput Assays for Locus-Wide IGH Genotyping in Clinical Populations.

Background.

The genomic structure of IGH is well-known to vary considerably between individuals 9-12,59,60, however the full ˜1 Mb IGH V, D, and J gene regions (excluding IGHC) have been sequenced only twice, once by Matsuda et al. 61 using a mosaic of three different large-insert clone libraries, and more recently by our group15 from a single chromosome. It is now appreciated that as many as 29 of the ˜54 functional IGHV gene loci occur in CNVs3, including variants as large as 75 Kb in length15,59 (FIG. 30); CNVs extend into the IGHD and IGHC regions 13,55,62. IGH genes also exhibit significant allelic variation, with some genes having >20 known alleles8,63. Taken together, this puts IGH diversity on par with the most polymorphic human loci, such as HLA4. Notably, locus-wide mapping of haplotype diversity in HLA has been critical for understanding its role in disease risk and therapeutic response64-67. Early candidate gene approaches associated IGH variants with susceptibility to both infectious and autoimmune diseases 68-70. However, likely due to inherent difficulties in assaying the locus 71,72, more recent genome-wide studies have failed to replicate these associations3. Indeed, our analyses have shown that commercial SNP arrays poorly represent known IGHV coding variants and CNVs 3,15; for example, the Immuno-array BeadChip 73 includes only 5 SNPs for the entire IGHV gene region, which harbors 1000's of polymorphisms. IGH also presents considerable challenges for standard short-read approaches; as a case in point, the 1KGP49,50, with the goal of characterizing all common human genome variants, does not claim accuracy of SV calls in the region 49,50.

Furthermore, the IGH allele database run by The Immunogenetics Information System (IMGT; www.imgt.org)63 is far from complete 3,13,15,74,75, and some curated alleles are disputed 74. This is likely the result of sampling only select loci in cohorts of limited size and geographic range (i.e., predominantly Europeans). This negatively impacts the analysis of Ab repertoire data (e.g., distinguishing SHM from germline) 76, and can impact clinical diagnostics 77. Demonstrating ethnic bias, our recent resequencing in IGH identified 10 new alleles, all from Asian or African individuals 15, and a recent study in 28 indigenous South Africans reported >120 new alleles not found in IMGT75. Methods for interrogating expressed Ab repertoires 78,79 have allowed the inference of germline IGH variation and CNVs 14,76, highlighting even more new alleles; however, these data cannot be deposited into IMGT, and are limited to coding variation. In fact, knowledge of variation in regulatory regions, including recombination signal sequences (RSSs), is even more limited than in coding regions, and many IGH regulatory regions are yet to be defined because of the incomplete genomic references available. In order to correctly perform association analyses between IGH polymorphism and the Ab repertoire, current data suggest that all types of variation, including CNVs and SNPs in IGH coding and regulatory regions will be important to ascertain 3,13,14,19. The most effective approach for assaying IGH variation is to perform direct genotyping experiments capable of capturing locus-wide genetic variation at nucleotide resolution.

Aim 1.1: Assembly of New IGH Haplotypes and Characterization of Diversity in the Human Population

Aim 1.1.1: Constructing 18 IGH Haplotypes Using Fosmid Libraries Derived from 9 Ethnically Diverse Individuals.

To accurately reconstruct complete IGH haplotypes, we will utilize a well-established human fosmid clone-based resource 80-82. These libraries were constructed from 4 Africans, 2 Asians, and 2 Europeans, and one individual of unknown ethnicity. 1-2 million fosmid clone-end reads per library have been Sanger sequenced and mapped, allowing for selection of clones comprising individual haplotypes 83. Each ˜40 kb fosmid clone represents DNA derived from a single allele. We have shown that a clone-by-clone assembly approach resolves complex IGH regions without the collapse of paralogous regions that can occur in standard shotgun-based WGS assemblies 15. First, fosmid tiling paths across the IGH region will be generated based on fosmid end-read mapping. ˜750 fosmids will be picked for sequencing. These data will be assembled using a modified version of our human genome assembly pipelines. In short, we will separately assemble each 40 kb fosmid (FIG. 31); these assembled fosmids will then act as an extremely long and accurate allele specific read. Variants identified on fosmids from overlapping genomic intervals will be phased, using HapCut, to yield the final assembled haplotypes for each individual 84. The data demonstrates that this long-read approach can successfully assemble, detect SNPs/CNVs, and phase the entire IGH region53. Once finalized, assemblies will be submitted to GenBank, and we will work with the Genome Reference Consortium to incorporate them into the reference assembly as alternate haplotypes.

Aim 1.1.2: WGS Long-read sequence analysis in 4 ethnically diverse trios. As part of HGSV and GIAB projects, genomes from four human trios have undergone WGS with long-reads, including individuals of Yoruban, Puerto Rican, Han Chinese, and Ashkenazi populations. Once our initial fosmid IGH haplotype resource is constructed, we will use it to extract data from these WGS resources to reconstruct 16 unique haplotypes from the parents of these trios. We will perform a two-step strategy using hybrid de novo assembly and phasing, combined with iterative reference mapping to our completed haplotype set. This will allow extension into unknown sequences and maximize new haplotypes.

Aim 1.1.3: Building a Comprehensive Allelic Database for IGH Using Targeted IGHV Sequencing and 1KGP variants, and Constructing a Population Reference Graph (PRG).

To identify alleles that are unique to a given ethnic group, many samples are required. We will take two approaches to supplement our haplotype maps constructed above with additional variation in IGH coding regions. We will first use an established method for targeted genomic IGHV gene amplification and MiSeq sequencing (300 bp paired-end reads, providing sufficient sequence information to resolve even highly identical paralogs) 75 in 288 ethnically diverse samples from the United States (African American, n=72; Asian, n=72; Hispanic, n=72; Caucasian, n=72; ). We will also screen for SNPs in coding and regulatory regions of IGH genes by mapping 1KGP 49,50 raw reads to our more complete set of IGH reference sequences. Identified variants from both will be validated by cloning and sequencing from gDNA of individuals from which the variants were initially identified, as required by IMGT for submission 8,63.

To circumvent limitations of single ‘linear’ genomes, the genomics community has increasingly moved towards “graph” genomes that represent haplotypic diversity from an entire population 85. Individual haplotypes are represented as a path in the graph, as opposed to inferred differences against a single reference. The PRG, recently applied to HLA, is a promising approach for analyzing hypervariable genomic regions86. A schematic for the construction of the PRG is shown in FIG. 34. One first aligns existing reference and haplotype sequences to one another (FIG. 34). Here, this will include GRCh37, GRCh38, and our assembled fosmid/WGS haplotypes (Aim 1.1.1, 1.1.2). This multiple sequence alignment is converted to a graph by collapsing highly similar aligned segments (FIG. 34). Next, variants from targeted IGHV resequencing and 1KGP analysis (Aim 1.1.3), as well as currently catalogued IGH alleles from IMGT and elsewhere, can be incorporated to valid paths in the graph. This combined representation of haplotype and variant data will allow assay designs in Aim 1.2.1 and provide the foundation for individual diploid IGH haplotyping in Aims 1.2.2 and 1.3. A IGH PRG based on GRCh37 and GRCh38 haplotypes shows that it can resolve known SVs/CNVs and capture SNPs/indels (FIG. 34).

Aim 1.2: Developing an Informed Custom Genotyping Platform for IGH

Aim 1.2.1: Designing High-Throughput Assays for Locus-Wide IGH Genotyping in Clinical Populations.

Sequence capture will be performed using a custom Nimblegen solution-based SeqCap EZ Choice Library. We will iterate on our design (Data) by including all known IGH sequences, supplemented with those derived from Aims 1.1/1.2. Based on our surveys of IGH variation15, Aim 1.1 will uncover several 100's of Kb of new IGH sequence. Prior to sequence capture, two separate sequencing library protocols will be used on each DNA sample, resulting in libraries with two different insert sizes: ˜800 bp and 6-8 kb. Construction of both libraries involves shearing, end-repair, A-tailing, and ligation of bar-coded sequencing adapters that allow multiplexing of samples; the larger library prep also includes an additional modified amplification step for increasing enrichment of larger fragments. We will employ a complimentary sequencing approach, using paired-end 300 bp reads on an Illumina MiSeq for the smaller ˜800 bp libraries, and a PacBio Sequel for long-read sequencing of the larger 6-8 kb libraries. Based on our data, this dual sequencing approach will allow for high confidence genotyping which optimally leverages the respective advantages of both platforms: highly accurate short reads allow high confidence genotyping of SNPs and short indels, while long PacBio reads are able to span stretches of non-unique sequence, accurately resolving duplicated regions, repeats, and SVs (Data). For clinical genotyping (see Aim 1.3.3), we will pool barcoded libraries from multiple individuals (n=24, MiSeq; n=4, PacBio) prior to sequencing. All QC will be done using custom pipelines based on current “best practices”. We will first perform genotyping of the IGHV, D, J and C regions in our nine IGH haplotyperesolved samples, to confirm we can recapitulate variants present in the assemblies generated in Aim 1.1.1; if modifications are required, they will be made at this stage, prior to genotyping in clinical samples.

Aim 1.2.2 Providing an End-to-End Pipeline for IGH Allele Assignment and Inference of Individual IGH Haplotypes Using the PRG.

After targeted capture, reads will be mapped to a custom reference/PRG enhanced with IGH haplotypes and variants identified in Aim 1.1. When reads map to only a single allele, or to a pair of dissimilar alleles, allelic assignment will be trivial. In hyper-variable SNP regions we will extend known methods for determining the most likely pair of alleles at a given locus 87, leverage the PRG, as well as long-read data. As stated earlier, threading samples through a PRG (in which similar variation may have already been observed) makes complex variation in highly polymorphic regions easier to detect. Each individual's short-read data will be first collapsed into a simplified form in which k-mer (a substring of DNA of length k) frequencies will be projected onto the PRG (FIG. 34). Next, a Hidden Markov Model (HMM) will be used to identify the maximum likelihood haplotype paths in the graph. These paths then act as new “reference” sequences (FIG. 34). Sample reads are remapped and new variation is discovered using standard variant calling algorithms and iterative refinement of the haplotypes (FIG. 34). Additional approaches utilizing paired-end, split reads and read depth, can further bolster CNV calls and determine breakpoint junctions88. These will be evaluated via haplotype consistency checks relative to the PRG. In addition, we will perform local assembly and phasing using both MiSeq and PacBio read data. Paired-end reads have improved ability to link distal SNPs89, and when joined with PacBio long-reads, phasing and haplotype assembly are further simplified. Ultimately, SNPs/CNVs will be annotated based on their genomic position in relation to coding and regulatory sequences (e.g., RSSs, promoters, and spacer sequences), and IGHV, D, J, and C gene allele calls will be made. Together these data will provide a fully annotated set of haplotypes that can be compared across individuals and ethnicities; eQTL information from Aim 3 will also later be incorporated as annotations. Once validated, this platform will be made publically available.

Aim 1.3 Genotyping a Diverse Cohort of Healthy Adults, Including Seasonal Influenza Vaccinees.

Locus-wide genotypes will serve as a basis for establishing connections between IGH genetic variation, Ab features and clinical outcomes in the context influenza vaccination. We will screen neutrophil DNA in two cohorts collected at DFCI (FIG. 29): 138 healthy multi-ethnic American seasonal influenza vaccinees (cohort 1); and 45 healthy donors of unknown ethnicity (cohort 2, used in Aim 3.1). After additional new haplotypes and variants are identified and integrated into the PRG, we will partition cohort 1 by ethnicity (African American, Asian, Caucasian and Hispanic) to generate metrics for SNP, CNV and IGH gene allele frequencies, and compare between the four ethnic groups using Fst to test for differentiation. Additionally, we will compare general diversity indices between ethnic groups and make the first estimates of locus-wide linkage disequilibrium (LD). Genotypes collected from targeted IGHV sequencing (Aim 1.1.2) in 288 additional individuals, which include overlapping ethnicities (Aim 1.1.3), will also be included to increase population sizes for analyses within IGHV genes. Without wishing to be bound by theory, we will see ethnic-specific differences based on our previous investigation of IGH variants in Europeans, Asians and Africans 15,28. These will be considered when doing eQTL analyses in Aim 3 and will represent the first locus-wide population genetics study of IGH.

Results:

Given our expertise in long-read sequencing and fosmid assembly we have full confidence that Aim 1.1.1 will yield high-quality, full-length assemblies for the IGH region. By evaluating the relative mappability of short-read data by population in our PRG, will not only expand the corpus of known IGH variation, but we will have high-confidence in which populations are well represented in our dataset and how robust our model is for subsequent assay design and analysis. Importantly, we also will uncover additional new sequence from the sequence-capture as these approaches often obtain ‘off-target’ overhang sequences at the boundaries of capture intervals. In the case of small insertions and rearrangements internal to the IGH region, we will be able to cluster reads by their ‘on-target’ mates, and pair with PacBio long-reads to perform local assemblies, to uncover new sequences present in clinical samples. This composite mapping, CNV integration, and targeted assembly approach will lead to robust characterization of the IGH locus in the study cohort, and result in new insight into the population genetics of the region.

Alternatives:

As consensus calling for PacBio sequencing improves and costs fall with the release of the higher throughput “Sequel” system from PacBio, of which MSSM has two machines, we may eliminate the necessity of hybrid short-read sequencing. Currently, alternative long-read approaches (Oxford Nanopore, TruSeq, LFR) are either not as cost-effective or do not have the continuous read lengths to resolve complex structures 90,91. However, these technologies are still early in development and may be reconsidered. Additionally, a recent technology by 10× genomics has ability for resolving very large SVs (>100 kb) and phased haplotypes (>10 Mb). MSSM has early access to 10× and our group is actively working to prototype its use. On the informatics side, while we will use the approaches described, we will continue to evaluate new assembly and graph genome methods, which may be used in embodiments herein. Some individuals will likely possess rare IGH CNVs not detected in Aim 1.1, and will be confounded by artifacts resulting from pull-down coverage variation. If it appears that too much variation is observed in CNV modelings, we will also utilize assays optimized for CNVs, such as Taqman qPCR and Nanostring, which we have previously demonstrated effective in IGH (ref 15,28) and other structurally complex regions (92). We are confident the majority of IGH will be amenable to assembly and genotyping by our capture-sequencing and analysis methods. However, if unsuccessful, we will focus on local assemblies of IGH coding and regulatory sequences, which will still yield a rich resource for the community.

Aim 2. Phenotypic Characterization of Key Signatures of the Dynamic and Diverse Ab Repertoire in a Multi-Ethnic Cohort Before and After Seasonal Influenza Vaccination.

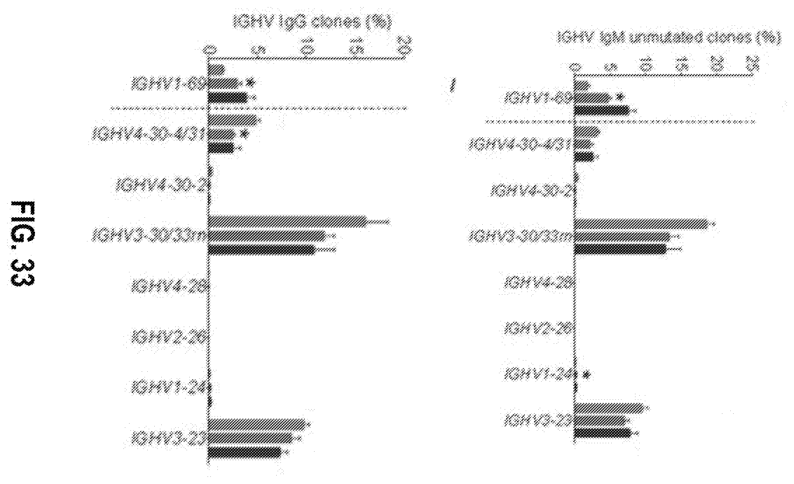

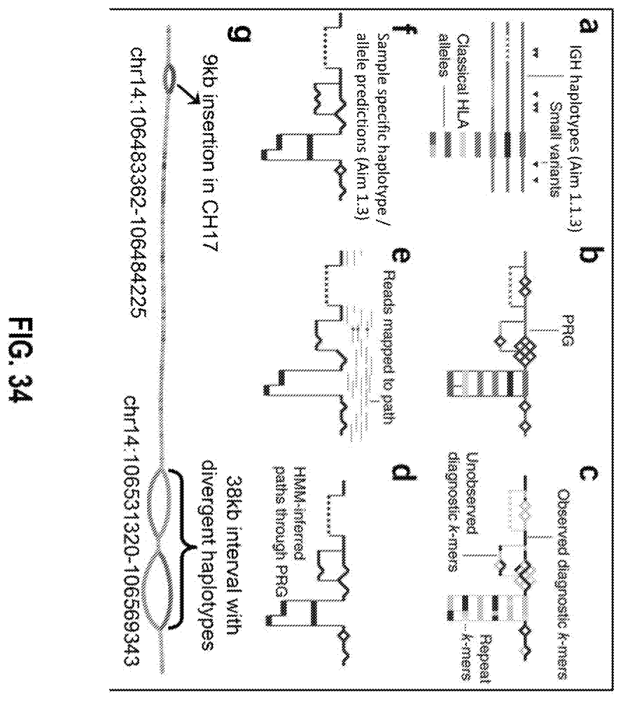

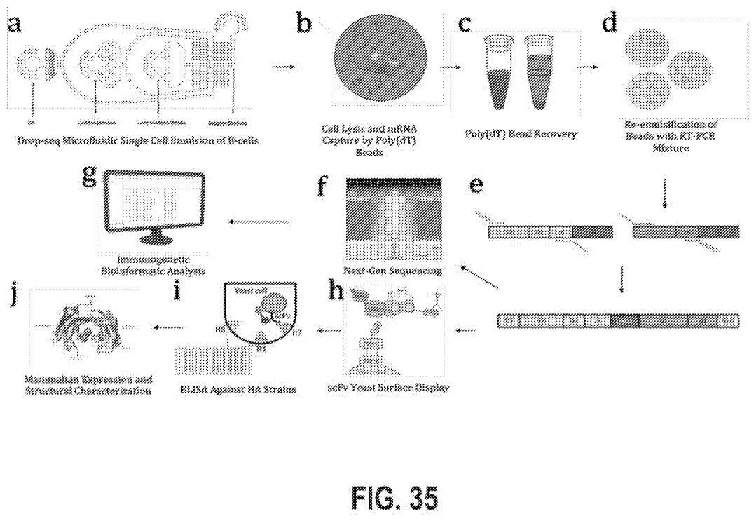

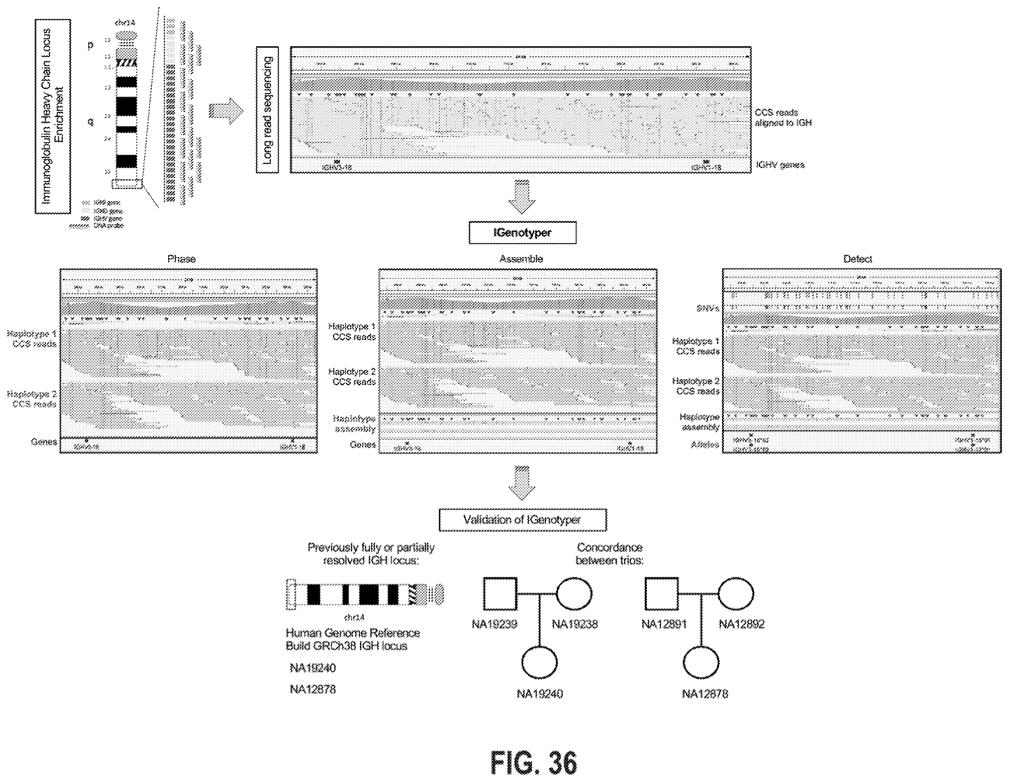

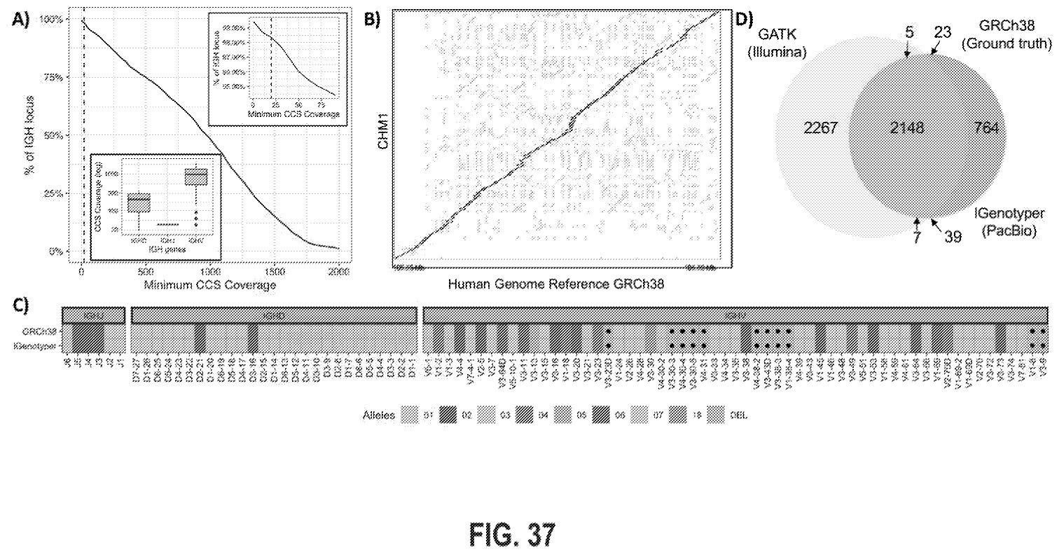

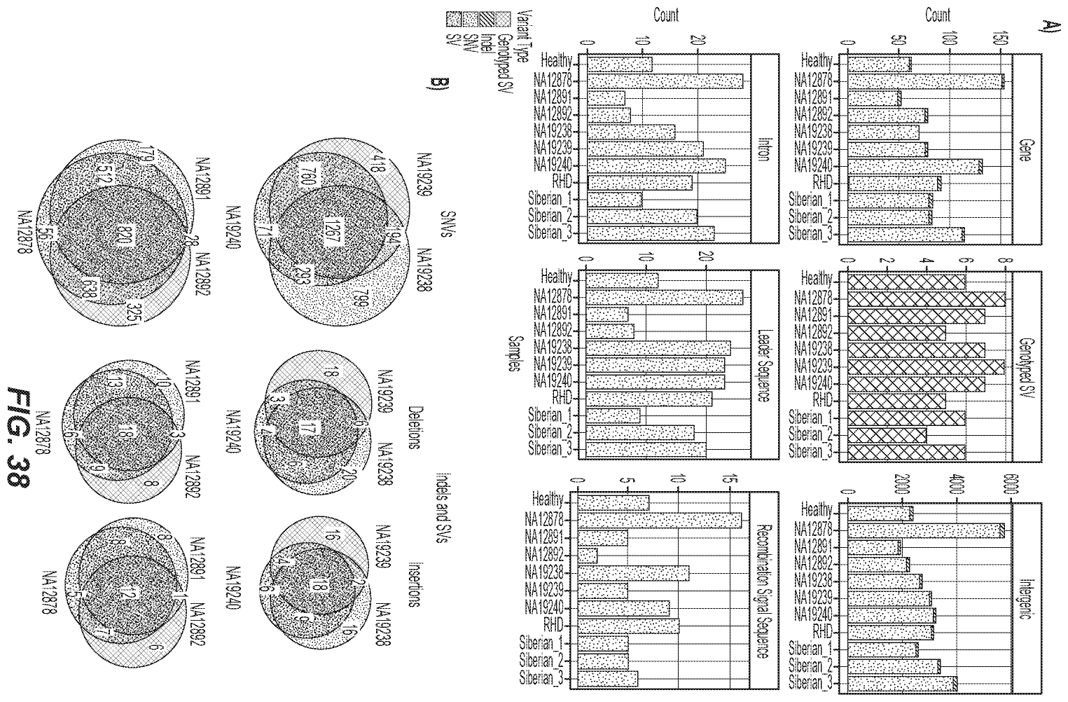

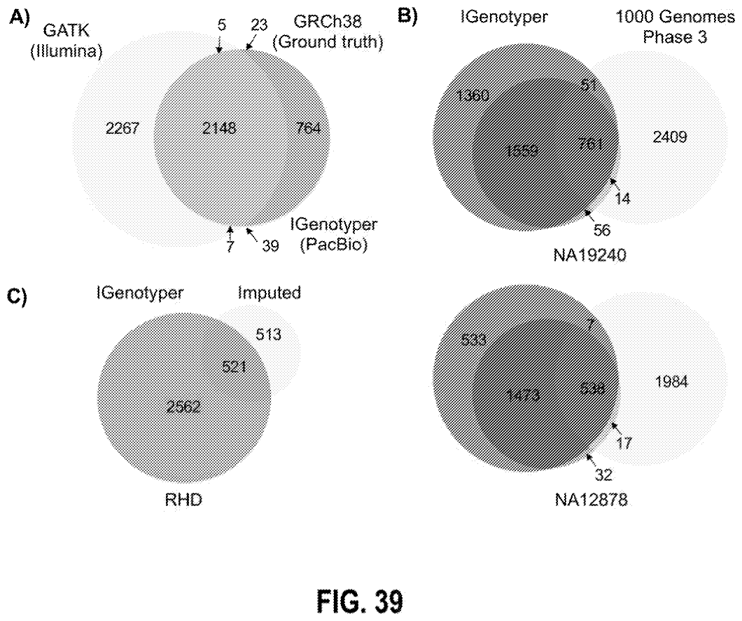

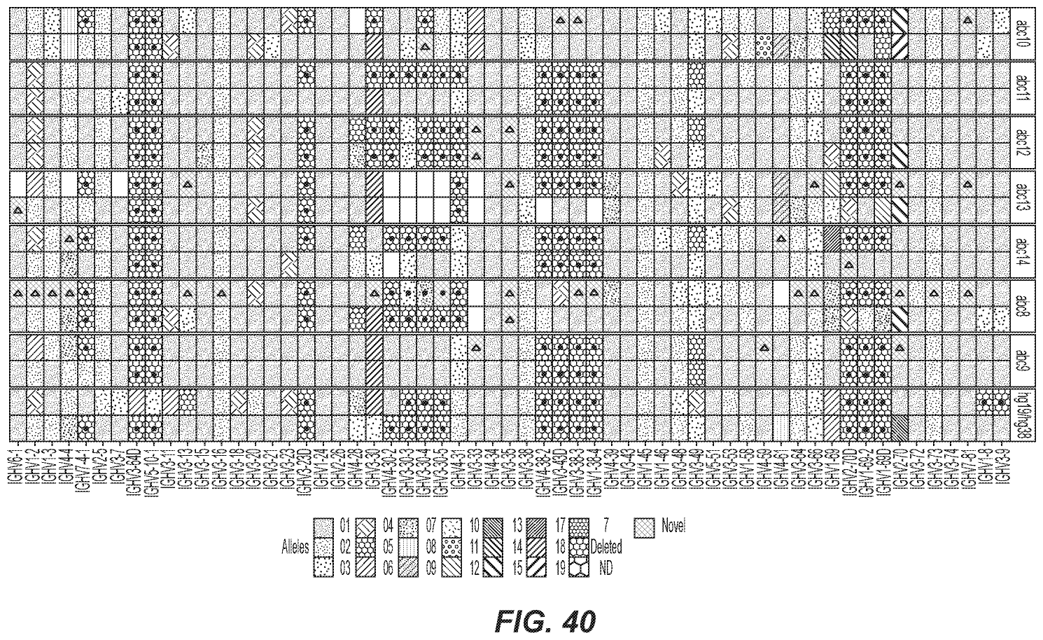

The structural bases for the generation of antibody (Ab) diversity has been the subject of numerous studies that have led to the contemporaneous view of the heavy chain CDR-H3 is dominant in determining binding specificity 93,94. However, recent analyses of the growing number of available Ab structures indicate that although CDR-H3 contributes more to antigen (Ag)-binding energy than other CDRs, CDR-H2 typically forms the same number of interactions with Ag95. Computational analysis of known Ab-Ag structures have shown that different heavy (H) and light (L) chain CDRs contain a median of 6, 6, and 4 contact residues in H3, H2, and H1, respectively, and 5, 1, and 3 contact residues for L3, L2, and L195. The overall percentage of energetically important Ag-binding roughly follows this same rank order circa 31%, 23%, and 14% for H3, H2, and H1, respectively and 14%, 6%, and 13% for L3, L2, and L1, respectively. Therefore, up to 40% of the amino acid contacts and energy can be attributed to the CDR-H1/2 amino acids. Moreover, only certain positions in the CDRs frequently make Ag-contact whereas other residues only appear to contribute indirectly by shaping the binding site (e.g., F54 at the tip of the IGHV1-69 CDR-H2 loop, Data); particularly in CDR-H2, it is likely that many of these residues are germline encoded and polymorphic at the population level. The importance of CDR-H1/H2 and individual amino acids within these regions establish a basis for V gene biases in the Ab response and other associated repertoire signatures (FIG. 28), and that these have an identifiable underlying IGH germline genetic component. As a first step toward defining links between IGH polymorphism and features of the Ab response, we will use a new pipeline (FIG. 35) to capture phenotypic and functional Ab repertoire biases in an influenza vaccinee cohort.

Aim 2.1.—Capturing the Phenotypic Diversity of the Expressed Ab Repertoire.

Phenotypic readout of IGH genotypic changes can take several forms due to CNVs and SNPs in coding and non-coding regions. This information can be captured by quantitation of Ab transcription levels in circulating B cells and in the titers and types of specific serum Abs (Data). We will use NGS short-read sequencing and established bioinformatic pipelines28 to perform quantitative analysis of circulating expressed IgM and IgG Ab repertoires from our influenza cohort 1 (FIG. 29). Naive CD27−IgM+ (naive), CD27+IgM+ (marginal zone) and CD27+IgG+ switch memory B cell populations will be analyzed in each blood sample through use of different reverse priming (IgM/IgG) and bead separation (CD27) strategies. IgM and IgG repertoires will also be sequenced from a single blood draw of 45 additional healthy donors (cohort 2), which will be used to supplement baseline repertoire eQTL analysis in Aim 3.1 for increased power. Basic IgM and IgG repertoire features, such as IG gene usage, V(D)J recombination frequencies, CDR characteristics, clonal diversity, and SHM will be catalogued here for use in Aim 3. Neutrophil genomic DNA will be isolated and banked for use in Aim 1.3 and Aim 3.

To advance beyond current Ab repertoire profiling technologies of sequencing only unpaired VH genes, we will develop a new pipeline that will allow the in situ assembly of bar-coded cognate VH-VL pairs from single B cells of each sample and their high-throughput analysis by NGS (FIG. 35). This will be an important experimental component for improving our ability to identify and catalogue key sBnAbs signatures (see herein). Optimized PCR primer design for these experiments was carried out by querying the IMGT database for all V region sequences of all functional germline IG genes96. Each new primer was tested individually via PCR to validate its functionality. Reverse J and C region primers were designed in a similar fashion. For the pipeline, the primers will be further modified with extensions that serve three functions (FIG. 35): to create an overlap extension (OE) that links heavy and light chains to form functional scFv cDNA via OE RTPCR97; to append an in-frame barcode in the end of the light chain sequence for sample identification for NGS; and to provide extension sequences which will allow downstream yeast display applications using our engineered version of the pCTCON2 vector (Aim 2.2) 98. The extensions can also be used as common priming site for primers that will serve to drive amplification of only sequences that have been extended.

A Drop-seq microfluidic device 99 will be used to create water in oil emulsion droplets of single B-cells. Each droplet will contain a lysis buffer and magnetic poly(dT) beads for capture of mRNA. Devices will be fabricated using Harvard Medical School Microfabrication core facilities. Fabrication involves using a bio-compatible, silicon-based polymer, polydimethylsiloxane (PDMS) via replica molding using the epoxy-based photo resist SU8 as the master. The PDMS devices will be rendered hydrophobic. Detailed protocols for the fabrication of the drop-seq microfluidic device and for creating emulsion droplets of single B cells can be found at the core website 100.

Aim 2.2. Identification and quantitation of anti-influenza sBnAbs by yeast display and HA sorting. Our studies will also focus on sBnAbs against the highly conserved HA stem. We have bifurcated use of the samples (FIG. 35) to allow in frame cloning for yeast display and genetic and functional identification of sBnAbs. scFv-yeast libraries will be subject to FACS-sorting for their ability to simultaneously bind multiple fluorochrome tagged influenza HA trimers. Studies will be performed to optimize initial sets of trimer pairs for FACS-sorting including contemporaneous circulating influenza A H1/H3, non-contemporaneous circulating influenza A group 1 (H5) and group 2 (H7) and influenza B (Victoria and Yamagata) strains. The recovered yeast will be amplified and resorted for 2-3 rounds before unique clones are identified by individual colony DNA sequencing and functional interrogation is performed by multiplex ELISA-based meso scale (MSD) detection using 384-well plates onto which each well is spotted with 6 different HAs and a control protein. Further epitope mapping can be performed with HA-stem competition assay with validated sBnAbs such as 311433. Positive hits from these screens will be used to query the scFvs genes against our NGS datasets for phylogenic tree analysis. Abs that show broad binding will be further evaluated through direct cloning into scFv-Fc plasmids for mammalian cell expression followed by purification, kinetic binding studies and virus neutralization assays.

Aim 2.3. Algorithm Development to Expand and Refine Anti-Influenza sBnAb Molecular Signature Database and Predict Host Exposure to the HA Stem Epitope at the Molecular B Cell Level.

We will use our “validated” anti-influenza sBnAbs as a training set to query for additional signatures within each subject's Ab repertoire. New signatures will be experimentally validated for binding through Ab gene synthesis, mammalian cell line based scFv-Fc expression and HA binding/virus neutralization studies. We will extend this signature database derived from HA sorted Ab-yeast clones by developing machine learning algorithms to identify sBnAb signatures. Molecular sequences that are compiled from our training and HA-sorted clones will be used to develop models of the immune response to the highly conserved HA stem epitopes. Artificial neural nets (ANN), HMMs, support vector machine, and random forest machine learning algorithms will be evaluated for suitability and accuracy of modeling (101-104). For training, antibody variable region sequence, or parts of the variable region such as the CDR sequences or the “paratome”105 residues from the cloned anti-influenza sBnAb genes, will initially be used. These signatures should be absent from influenza naïve individuals that make up a portion of our cohort.

We will also conduct serologic studies in our cohort for Abs of interest. Serially diluted plasma samples will be tested by MSD ELISA for binding to 6 different HA proteins included influenza A group 1 H1, H2, and H5 and group 2 H3 and H7 strains and an irrelevant protein BSA. Anti-influenza sBnAbs titers will be determined by ELISA competition by plasma for mAb3I14 binding to H3. Plasma samples with broad hetero-subtypic HA binding activity will also be tested for H1, H2 and H5 pseudovirus and H3, H7 virus neutralization activity. These serologic data will be compared to the absolute quantity of sBnAb clones that are identified from each blood sample. This higher-level phenotypic data will also be assessed for associations to IGH polymorphism in Aim 3.2.

Results:

For Aim 2.1, the expression levels of IGH V, D, and J genes, and additional repertoire features during the vaccine response will be obtained in these studies for naïve and memory cell populations as planned. Linkage of the cognate VH-VL pairs will also be obtained, improving our ability to make lineage assignments. Given variable efficiencies of droplet generation, our estimation is that we may capture 600,000 cells during a 1.5 hr run using our initial flow and cell concentration parameters. Flow rates, oil mixtures, and lysis buffers have been previously optimized by members of the McCarroll research group and the staff of the HMS Microfabrication core to ensure stability of each droplet and complete lysis of each cell. Using dual syringe pumps set to flow lysis/bead buffers and cell suspension at 4000 uL/hr while a second syringe pump set to flow oil at a rate of 15000 uL/hr, we anticipate that oil droplets will be collected for 1.5 hrs. Flow rate optimization will be conducted, which may positively impact our throughput. Droplet creation will be assayed by replacing lysis buffer with PBS and trypan blue dye. Droplet size, uniformity and the presence of a cell will be assayed using a hemocytometer and light microscopy. Under lysis buffer conditions, the cell membrane should not still be visible after droplet collection. The scFvs in Aim 2.2, each expressing an anti-influenza BnAb derived from a single B cell will form the functional nodes that we will use to assess clonal diversity and expansion in the NGS dataset. This will allow us to expand our initial database of anti-influenza BnAbs molecular signatures that can be further interrogated in Aim 3. For Aim 2.3, the dataset of confirmed and epitope mapped sBnAbs will be used to identify additional binders with variegated sequences and enhance the diversity of the training data. We will initially query the expressed Ab repertoires of the same subject to which the sBnAbs were derived. Identified variegated VH sequences will be confirmed using our previously established methods28. These algorithms will continue to be refined as additional datasets are queried, including the analysis of the whole study cohort.

Alternatives:

Given the technical complexity of a multiplexed RT-PCR in emulsion droplets (Aim 2.1), we will initially achieve a scFv recovery rate for 10-20% of all cells processed. We also will yield >95% accuracy in pairing for those recovered. Experiments will be run to evaluate the frequency of non-native pairings. This will include using populations of cells from 2 or more expanded single B cell cultures, or immortalized lymphoblast cell lines. After sequencing, the percentage of heavy and light chains which have been correctly paired will be a measure of the accuracy of mRNA capture and cognate pairing. If false pairing is evident, modifications to flow rates, oil mixtures, and droplet handling have been shown in similar Drop-seq trials to nearly fully correct for errors. A secondary measure that could be taken to investigate this issue would be to expand B-cell populations into technical replicates. Each B-cell population would then be sequenced. The sequences that exist in both replicates are theoretically true cognate pairs. Off target priming during PCR can be assayed by analyzing a sample of the product via agarose gel electrophoresis. An alternative approach to Aim 2.2 could be to perform HA sorting of single B cells followed by in vitro expansion as we have reported but the throughput is not nearly as high so we prefer the approach that we have outlined. For Aim 3.3, we have developed an important contingency plan to build the database if required. This would involve an in vitro directed evolution strategy using yeast display of selected epitope mapped anti-influenza sBnAbs for which only VH of the cognate VH/VL pair will be mutated by error-prone PCR as we have previously reported (28). FACS sorting with HA trimer pairs will be to rescue the mutant VH genes that still bind HA. These variegated VH sequences would be added to the training set to further query the test expressed Ab repertories.

Aim 3. Characterizing Functional IGH Haplotype Variation Associated with Variability in the Expressed Antibody Response in a Cohort of Healthy Adult Seasonal Influenza Vaccinees:

Background.

There is now strong support for the importance of germline IGH polymorphism in determining the naïve and Ag-stimulated Ab repertoire. Early work in MZ twins provided initial evidence that the Ab repertoire was under genetic control 106. With the advent of NGS-based deep repertoire sequencing, this has now been investigated at greater resolution. Several recent studies of Ab repertoire data in MZ twin pairs revealed that IGHV, IGHD, and IGHJ-gene usage, as well as CDR features in naïve repertoires were much more highly correlated between genetically identical twins than between unrelated individuals 16-18. Intriguingly, signatures in Ag-experienced repertoires partly reflected those observed in the naïve, indicating that although memory B cell populations are affected by environmental exposures, they represent sampling events from fairly static, genetically-determined naïve repertoires 16-18. Analyses of repertoires in unrelated individuals have also demonstrated that D-J pairing frequencies are not random, and by inferring IGHD-J “haplotypes”, it was shown that individuals carrying deletions of particular IGHD genes had more similar D-J recombination patterns 55. Additional examples directly linking IG polymorphisms to IG gene repertoire features also exist, revealing effects of CNVs, and SNPs within IG coding and regulatory regions (Data) 9,28,107-111, including those with relevance to disease and clinical phenotypes 28,107,110,111. However, all studies conducted to date have been based on limited datasets, restricted by the number of IG variants tested, cohort size, and/or the use of crude measurements of IG gene usage estimated by methods other than repertoire sequencing; thus, more comprehensive investigation of IG germline effects on the Ab response is warranted.

Aim 3.1. Characterizing Functional IGH Germline Variants with Effects on Baseline Ab Repertoires of Healthy Adults from Multiple Ethnic Backgrounds.

We will investigate the effects of IGH polymorphism on baseline Ab repertoire features from three B cell subsets with different functional profiles pre-vaccination. This will provide the first catalogue of IGH functional germline variants with relevance to many disease contexts, and provide a starting point for investigating the molecular mechanisms underlying IGH germline effects on the Ab response. Existing algorithms for interrogating expressed Ab repertoires allow for statistically valid comparisons between repertoires, including the accurate estimation of classic Ab repertoire characteristics that have been associated with underlying genetic factors16-18,28,55,110(Data) 28. We will take all IGH genotypes from 183 adult individuals (cohorts 1 & 2, Table 2) and perform a cis-eQTL analysis using the following pre-vaccination baseline features of Ab repertoires from unmutated IgM naïve, marginal zone, and IgG class switch memory B cells as quantitative traits: (i) IGHV-, D-, and J-gene usage frequencies; (ii) IGHV, D, and J allele-specific usage; (iii) V-D and D-J recombination frequencies; and (iv) VH-VL cognate pairing frequencies. Basic cis-eQTL analysis will be performed using the GGtools R package 112, which fits a generalized linear model (GLM) to the data, with genotype as the predictor variable. In order to improve robustness and account for relevant covariates in this analysis, eQTL models will incorporate age, gender, and ethnicity (based on self-reported data and estimates derived from principal component analysis of IGH genetic variation). In order to account for additional sources of hidden variation in gene expression measures (e.g. batch effects, environmental variables) that can confound eQTL association analysis, we will apply PEER 113. Briefly, PEER first infers hidden covariates influencing gene expression measures as well as their weight. PEER then subtracts the component of the hidden covariates and produces a residual gene expression matrix that can be used for association analysis. This approach has been shown to considerably reduce false-positive associations, and results in an overall improvement in statistical power by reducing noise. False discovery rate will be used to control for multiple testing 112. In addition to individual cis-eQTLs, we will look for gene-gene interaction effects (e.g., testing for effects of IGHV3-30 polymorphism after conditioning on IGHV 1-69 genotypes), and long-range haplotype effects. Given we have previously identified combined effects of IGH gene CNV and allelic variants (Data) 9,28, we will perform tests in CNV regions for effects of copy number changes of particular alleles. In addition, we will look for interactions between age and genotype, using an interaction term in a separate GLM analysis. Although analyses combined across all samples in our cohort will have the most power, this approach cannot discern population specific effects. Thus, we will also test for eQTLs independently within each ethnic background of cohort 1, allowing for comparisons between African Americans, Asians, Hispanics and Caucasians (Table 2). We will choose 5-10 functional variants with the largest effects for design of targeted Taqman qPCR assays for experimental validation, and cost-effective broad use in the Ab research community.

Aim 3.2. Identifying IGH variants that associate with variability in Ab repertoire signatures and circulating Ab titres post-vaccination. A major strength of cohort 1 (Table 1) is that it includes data across multiple time points pre-vaccination and post-vaccination within the same individuals. While associations characterized in Aim 3.1 provide insight into baseline repertoire features observed generally in the population, this sub-aim will investigate whether IGH polymorphisms can have effects on repertoire signatures (collected in Aim 2) more directly related to the functional Ab response following seasonal influenza vaccination. Using a cohort of 18 H5N1 vaccinees (Data) 28, we previously observed associations between IGHV1-69 variants and features in IgM and IgG repertoires post-vaccination, as well as serum circulating sBnAb titres. In this sub-aim, using data from three B cell subsets (naïve IgM, marginal zone IgM, and class switched IgG) at three time points (prevaccination, 7 days and 30 days post-vaccination), we will expand on our previous findings by conducting similar eQTL association analyses between all IGH germline variants and each of the following repertoire features at a per gene level: (1) numbers of highly expanded clones; (2) ratio of IgG/IgM gene usage (class switch frequency); (3) SHM frequencies; and (4) sBnAb precursor clone frequencies and sequence signature characteristics (as determined in Aim 2.3, μlacing emphasis on sBnAbs that are targets for vaccine design). Finally, we will also look for higher-level effects of the IGH germline on circulating titres of select Abs of interest identified in Aim 2.3. This will be done using the same GLM framework and covariates outlined above, and will also include secondary investigations of gene-gene, allele-specific CNV, age, and ethnicity effects. In addition, relevant to this analysis, past influenza exposure is known for a subset of cohort 1; we will also test for interaction effects between this factor and genotype on Ab repertoire signatures and Ab titres.

Results:

Nearly four decades since the study of IG genetics began, the role of specific IGH germline variants in Ab expression and function have not been comprehensively defined. This analysis will result in the first catalogue of functional IGH variants associated with features of the Ab repertoire. The results of Aim 3.1 will be useful to a growing community of immunologists using Ab repertoire sequencing. Given that the primary variants identified in this aim are those associated with baseline repertoire features (e.g., gene usage), this catalogue could provide useful a priori information for initial studies of IGH germline repertoire effects in other disease contexts of interest; especially considering that we and others have shown that IGH variants impacting the naïve repertoire can also have associations with other key signatures in Ag-stimulated repertoires 28,110. In addition, Aim 3.2 will provide genetic information for better understanding the Ab response associated with seasonal influenza vaccination. Specifically linking these data with knowledge of key sBnAbs that are targets of current vaccines (including an expanded list form our efforts in Aim 2.2 and 2.3), could provide actionable information for improving vaccination strategies beyond a one size fits all approach. More generally, paired with haplotype maps from Aim 1, these data will lay a foundation for the design of experiments to delineate the molecular mechanisms mediating genetic effects on the human Ab repertoire.

Alternatives:

Based on our cohort sizes, eQTL analyses will allow for even fairly subtle effects of IGH germline variation on Ab repertoire features, from gene usage to BnAb signatures. Power calculations suggest our primary eQTL analyses in Aim 3.1 (n=183) and Aim 3.2 (n=138) are well-powered, with an ˜85% and ˜70% probability of detecting SNPs/CNVs explaining just 10% of the variance in tested repertoire signatures. We concede that after partitioning by ethnicity, our power to detect small effects and gene-gene interactions decreases considerably. However, identification of variants with large effect sizes should still be possible. Particularly for germline variants linked to vaccine-associated signatures, these may be most important. For example, in our previous analysis of Ab repertoires in 18 H5N1 vaccinees, a single SNP was capable of explaining ˜60% of IGHV1-69 usage variation in the naïve subset; and this increased to ˜80% when CNV was also considered 28. Given the resolution at which we will be able to genotype IGH, multiple layers of haplotype information are likely to further improve our power to detect differences. In addition to Ab features for which we have already demonstrated effects of specific germline variants, we will also investigate associations with biases of V-(D)-J recombination events and VH-VL cognate pairing frequencies. IGH germline effects on such features will be minor. However, a recent study showed that effects on D-J recombination could be observed after partitioning samples by the presence of IGHD gene deletion haplotype55, even in a cohort of 25. Given our cohorts are larger, this investigation is worth the effort. Our results will demonstrate proof of principal for locus-wide IGH genotyping, which could be extended to IG light chain genes as a next step.

### Example 16

Abstract

There is a fundamental gap in our understanding of how germline variation in immunoglobulin (IG) heavy (IGH) and light chain (IGK; IGL) loci in the human population impacts the development of the functional antibody (Ab) response in health and disease. However, there is a growing appreciation that IG polymorphism contributes to variability in the Ab repertoire, indicating that the integration of IG genetic data has the potential to inform our understanding of Ab function in various clinical contexts. A critical barrier to progress has been that existing genomic resources for IG loci are lacking and poorly represent diversity found across human populations. IG regions are structurally complex, consisting of large segmental duplications, and are among the most polymorphic in the genome, with large copy number variants (CNVs), elevated nucleotide diversity, and population-specific haplotype variants. These complexities have long made IG loci difficult to study at the genomic and population level using standard high-throughput methods, with direct negative impacts on genetic disease association studies and more recently the analysis of expressed Ab repertoire data. As a result, our knowledge of human IG germline diversity (particularly in non-Caucasians) and its contribution to disease lags far behind that of other well studied immune loci. This highlights a direct need for publically available well-characterized IG haplotype references and accurate variant catalogues from diverse ethnic backgrounds to facilitate the design and integration of more accurate genotyping tools, analysis pipelines, and their interpretation. To meet this need, we have developed several robust approaches, which we will utilize here to establish critical community resources for the IG loci. We will first enumerate up to 16 novel IGH/K/L haplotype reference assemblies from an existing set of 8 fosmid libraries from individuals of African, Asian, and European descent. We will also use a novel multi-haplotype informed genotyping pipeline to profile IGH/K/L genetic variation in a cohort of 180 familial and unrelated individuals from these same three populations. This will represent the most comprehensive population survey of IG germline diversity, including descriptions of variable, diversity, joining, and constant gene variation, and locus-wide single nucleotide polymorphisms (SNPs) and CNVs, allowing for fine-scale assessment of variant imputation panels for disease association studies. Finally, to facilitate the utility of these data as long-term resources, all sequences, tools/methods, and analysis pipelines will be made publically available. We will work with established databases to ensure all sequences are deposited in both raw and annotated form. This will include the integration of assemblies into future releases of the human genome reference for use by the genomics community, as well as updates to existing germline gene/allele databases critical to expressed Ab repertoire analysis. This project establishes desperately needed genomic resources for the human IG loci, which will better serve the immunology community for years to come. These will stand as a foundation for future efforts to define the role of IG germline variation in Ab function, health, and disease.

Aims

Genes at human immunoglobulin (IG) heavy (IGH) and light chain (IGK, IGL) gene regions encode antibodies (Abs), critical components of adaptive immunity. These loci: span ˜3 MB of the genome; consist of hundreds of repeated, highly homologous sets of variable (V), diversity (D), joining (J), and constant (C) genes; and are among the most polymorphic in the genome, characterized by large gene-containing copy number variants (CNVs), elevated nucleotide diversity, and population-specific haplotype variation. Importantly, there is mounting evidence linking IG germline variants to inter-individual variability in Ab expression and function, including examples in infection, autoimmunity, cancer, and vaccine response. Together, these observations validate the use of IG genetic data to better understand the Ab response at the individual and population level, including applications in precision medicine. However, as demonstrated in other biomedically-relevant hyperpolymorphic gene regions, comprehensive profiling of IG germline variation in clinical populations will require both a baseline knowledge of population variability, and strong foundation of genomic resources for the design/application of genotyping tools, analysis pipelines, and their interpretation.

At present, existing genomic resources (i.e., reference assemblies and variant catalogues) for the IG loci are incomplete and poorly represent germline diversity across human populations. We and others have shown that this negatively impacts genetic association analysis and Ab expression sequencing data, standing as a critical barrier to studying IG variation in health and disease. We are well positioned to overcome this barrier, as we have developed new approaches for re-constructing full-locus IG haplotype assemblies and effectively surveying population-level genetic diversity utilizing long-read sequencing. The primary objectives of this example use these approaches to build upon and extend existing community resources by generating alternative full-locus reference assemblies and germline variant catalogues for the IG loci in ethnically diverse human samples. We will accomplish these objectives by pursuing the following two specific aims:

Aim 1. Construct a Comprehensive Set of Human Full-Locus IG Haplotype Reference Assemblies in Individuals of African, Asian and European Descent.

We will use our developed approach to enumerate new IGH/K/L haplotypes from available fosmid libraries for 8 diploid individuals of African (n=4), Asian (n=2), and European (n=2) backgrounds from the 1000 Genomes Project (1KGP), resulting in up to 16 complete high-quality reference assemblies, representing existing IG genetic variation across human populations. These haplotypes will be validated by orthogonal sequencing methods and datasets and fully annotated to catalog new genes/alleles, as well as structural and single nucleotide variation. Assemblies will be compared to gain initial insight into haplotype diversity features, and differences between the IGH, IGK, and IGL loci.

Aim 2: Construct an Accurate Population-Level IG Genotype Reference Database from Three Human Populations for Improved Disease Association and Ab Repertoire Sequencing Data Analyses.

We will leverage haplotype data generated in Aim 1 to further develop our existing sequence capture assay and analysis pipelines to target the IGH/K/L loci, which, when combined with a long-read sequencing and our improved IG reference assemblies, will allow for genotype-level resolution across 174 individuals from 1KGP/HapMap African, Asian, and European populations (including trios and unrelated samples). Our panel will provide genotype calls for locus-wide CNVs and SNPs, identification and annotation of IGH V, D, J and C genes and alleles and regulatory region variation. Together this will represent the largest population survey of human IG germline diversity to date, allowing for the evaluation of intra- and inter-population IG variation, and assessment of our variant resource to offer improved imputation efficiency for disease association studies.

Raw and annotated sequence data will be submitted to the NCBI SRA and GenBank, and all variants identified (SNPs and/or CNVs) will be deposited into dbSNP and dbVar. In addition, we will integrate newly constructed IG haplotypes into future releases of the genome reference assembly, and ensure all new genes, alleles, variation and haplotype information identified are made available in fully curated form.

The outcomes of this project will establish desperately needed improvements to genomic resources for the human IG loci, which will better serve the immunology and genomics communities for decades to come. Just as such resources have provided a strong basis for genetics research in other hyper-polymorphic loci, those produced here will provide a foundation for future work investigating the role of IG variation in the Ab response in health and disease.

### Example 17

We have developed a high-throughput approach for more comprehensively obtaining high-quality genotypes across an immunoglobulin loci, such as the immunoglobulin heavy chain variable (IGHV), diversity (IGHD), and joining (IGHJ) gene regions. To do this we utilize custom designed Roche NimbleGen SeqCap EZ Choice oligo panels that target, for example, IGHV/D/J gene containing regions of human chromosome 14. Capture panels have been designed using non-redundant loci/sequence targets curated by C. T. Watson et al. (2013), which account for all known insertion and duplication sequences (i.e., those that could be encountered in the human population) that are not currently represented by the available human reference assembly genomes. Using these custom oligo panels, we have implemented a modified protocol that pairs the Roche NimbleGen SeqCap EZ standard operating procedure to generate longer fragment libraries (5-10 Kb) to more fully leverage the use of Pacific Biosciences platforms for long-read sequencing.

We follow the Pacific Biosciences shared protocol, “Target Sequence Capture Using Roche NimbleGene SeqCap EZ Library”, with the following modifications:

1) For all AMPure PB clean ups (critical):
a. At sub-steps e. and g., add 1 ml of fresh (made that day) 70% ethanol instead of 200 μl.
b. At sub-steps h.-j., carefully remove ethanol, remove from magnet, and pulse spin in a mini benchtop centrifuge for 1 second. Then place on magnet and remove remaining ethanol with P-10 pipette set to 10 μl. If there is no visible ethanol pooled around or on top of beads (they should look glossy or matte, NOT cracked) add TE or H2O depending on requirements listed. The length of elapsed time to complete these steps should not collectively exceed 20 seconds.

2) Adapters and Blocking Oligos (optional):
a. Use Pacific Biosciences index adapters with a universal priming sequence in place of SeqCap Adapters.
b. Use Pacific Biosciences “PB UPS” oligo as a blocking oligo instead of SeqCap HE-Oligo Kit A and B.

3) For Shearing Genomic DNA (optional):
a. At step 3 in the protocol, spin twice in 1-minute increments, invert, and again spin twice in 1-minute increments.

4) For Cleaning and Concentrating Genomic DNA (optional):
a. At steps 1-10, use vacuum concentration instead of AMPure bead purification.
b. Poke 3 holes into Lo-bind tube cap (equally spaced within cap). Add 150 ul of sheared DNA to Lo-bind tubes. Vacuum concentrate to 30 ul final volume.

5) For Library Preparation of Size-selected Genomic DNA (optional):
a. At step 1, use 400 ng of DNA instead of 200 ng.
b. At step 2, use 10 uM of the annealed Pacific Biosciences indexed adapters with “PB_UPS”.
c. Incubate ligation mixture at 20° C. for 20-60 minutes, or 20° C. for 60 minutes and then 4C overnight.

6) For Library Amplification (critical):
a. For step 1, replace “Mixture of PCR Oligos 1&2 (50 uM each)” with “PB_UPS oligo, 50 μM”.
b. For step 2, PCR conditions, replace step 4 “Repeat Step 2, 6 times” with “Repeat Step 2-3, 9 times”.

7) For Post Amplification Cleanup (critical):
a. For steps 8 and 9, following AMPure bead cleanup, elute in 52 ul of water (instead of 27 μl), and using a powerful neodymium magnet (N38 or above) to isolate the AMPure beads to the side of the tube, remove supernatant. Discard tube with AMPure beads. Set the lip of a fresh Lo-bind tube on the top edge of the magnet. Place the pipette tip containing the supernatant across the lip of the Lo-Bind tube so that the liquid in the pipette tube is as close as possible to the magnet. Slowly pipette the supernatant into the fresh tube. Stop pipetting supernatant when there is 2-5 μl of supernatant in the tip. This supernatant will very likely have AMPure bead particulates and should be discarded.
b. After step 11, conduct quality control on the sample using the Agilent Bioanalyzer. If the Bioanalyzer trace does not resemble a sharp peak, and there is visible DNA below 5 kb, use the Sage Blue Pippin to size-select the DNA, using the same parameters used for the initial size selection of genomic DNA. If the total DNA quantity is below 1.5 ng, additional PCR cycles should be completed before the hybridization steps.

8) For Hybridization (critical):
a. Use PB_UPS oligo in place of the SeqCap HE Universal and SeqCAP HE Index Oligo.

9) For Amplification of Capture DNA sample (critical):
a. For step 2, in the PCR protocol, replace step 4 “Repeat step 2, 14 times” with “Repeat step 2-3 19 times”

10) For Post-Capture, Post-Amplification Cleanup (critical):
a. For step 8, elute in 52 μl of TE buffer (instead of 27 μl).
b. For step 9, using a powerful neodymium magnet (N38 or above) to isolate the AMPure beads to the side of the tube, remove supernatant. Discard tube with AMPure beads. Set the lip of a fresh Lo-bind tube on the top edge of the magnet. Place the pipette tip containing the supernatant across the lip of the Lo-Bind tube so that the liquid in the pipette tube is as close as possible to the magnet. Slowly pipette the supernatant into the fresh tube. Stop pipetting supernatant when there is 2-5 ul of supernatant in the tip. This supernatant will very likely have AMPure bead particulates and should be discarded.
Once SMRTbell sequencing libraries are constructed (i.e., capture protocol above is completed), libraries can be sequenced on the RSII, Sequel 1, or Sequel 2 platforms.
Once sequence data have been generated, we have developed an analysis pipeline to process sequence data and generate locus-wide genotypes and gene annotation summaries.
Steps to assemble and characterize locus-wide genetic variation in the immunoglobulin heavy chain locus (IGH):
   1. If reads are not in BAM format (e.g., in bax.h5 format), files are converted to BAM using SMRTanalysis [1].
   2. The following steps are coded into the software package, IGenotyper[2], developed specifically for this project
      a. The subreads within the BAM file are turned into CCS reads using the tool ccs[3].
      b. Reads are aligned to an in-house reference genome using BLASR [4];
      c. Single nucleotide polymorphisms (SNPs) are called using WhatsHap [5];
      d. SNPs are phased using WhatsHap [5] using aligned reads and SNPs called from step 2.c.;
      e. Similarly to the MsPAC methodology, as described here [6,7], reads are assigned to either haplotype 1 or 2 (or labelled ambiguous if unassignable) based on phased SNPs, and partitioned as such;
      f. Haplotype-partitioned reads from haplotypes 1 and 2 and ambiguous reads are binned into haplotype blocks, based on WhatsHap phased SNP calls, and where there is sufficient coverage;
      g. Each block is assembled using Canu [8];
      h. Original reads are aligned back to assembled haplotype block contigs (2.g.), and error corrected using Quiver [9].
      i. Statistics (tables and plots) on the sequencing run and assembly pipeline are produced
   3. For determining IGH gene/allele calls, the assembled contigs are aligned to the reference assembly, gene sequences are extracted from each contig, and gene/allele assignments are made via alignments to the IMGT germline database [10]. Additional CCS reads are also scanned for genes.
   4. Locus-wide SNPs are called by identifying alignment differences between assembled haplotype contigs and the reference genome assembly.
   5. Indels and structural variants (SVs) are called using MsPAC (based on multiple sequence alignment and a hidden Markov model).
   6. A set of 7 polymorphic SVs identified here [11] are genotyped using the CCS read alignments and assembled contigs
   7. SNP/SV genotypes and gene/allele call data can be used to assess the impacts on antibody repertoire features and associated clinical phenotypes.
[1] https://www.pacb.com/products-and-services/analytical-software/smrt-analysis/
[2] https://github.com/oscarlr/IG_clean
[3] https://github.com/PacificBiosciences/ccs
[4] https://bmcbioinformatics.biomedcentral.com/articles/10.1186/1471-2105-13-238
[5] https://www.biorxiv.org/content/early/2016/11/14/085050
[6] https://www.biorxiv.org/content/early/2017/09/23/193144
[7] https://www.ncbi.nlm.nih.gov/pubmed/31397844
[8] https://genome.cshlp.org/content/27/5/722
[9] https://github.com/PacificBiosciences/GenomicConsensus
[10] http://www.imgt.org/
[11] https://www.ncbi.nlm.nih.gov/pubmed/23541343

### Example 18

A Novel Framework for Characterizing Genomic Haplotype Diversity in the Human Immunoglobulin Gene Regions

The immunoglobulin heavy (IGH) and light chain loci comprise the building blocks of expressed antibodies (Abs), which are essential to B cell function, and are critical components of the immune system. The IGH locus, specifically, consists of >50 variable (IGHV), >20 diversity (IGHD), 6 joining (IGHJ), and 9 constant (IGHC) functional/open reading frame (ORF) genes that encode the heavy chains of expressed Abs¹. Based on the limited surveys conducted to date, >250 functional/ORF IGH gene segment alleles are curated in the IMGT database¹, and this number continues to grow²⁻⁸ (Wang et al. 2008). The locus is highly enriched for large structural variants (SVs). This includes deletions, insertions, and duplications of functional genes^{2,9-16}. Although limited, there is mounting evidence that allele frequencies at both single nucleotide polymorphisms (SNPs) and SVs within IGH vary among human populations^{3,16,17}.

The complexity of IGH has made it nearly inaccessible to standard high-throughput assays, limiting our ability to accurately and comprehensively screen IGH polymorphisms at the population-level^{18,19}. As a result, IGH has been largely ignored by genome-wide studies, leaving our understanding of the contribution of IGH polymorphism to antibody mediated immunity incomplete^{3,18,19}. While early candidate gene approaches did uncover IGH variants with associations to disease susceptibility, few definitive links have been made in the modern genomics era from the application of genome-wide association studies (GWAS) and whole genome sequencing (WGS) approaches^{18,20,21}. Moreover, little is known about the impact of genetic factors on the formation and regulation of the human Ab response, despite the fact that there is evidence that features of the Ab repertoire are heritable^{10,17,22-25} (Khosaka et al. 1996; Feeney et al. 1996).

To fully define the role of IGH variation in Ab usage, function and disease, many classes of variation, including both SVs, as well as coding and non-coding SNPs will be critical to resolve^{2,10,13,17,26} (Feeney et al. 1996). Although several approaches have been developed for utilizing either short-read genomic or Adaptive Immune Receptor Repertoire sequencing (AIRR-seq) data, variant calling and broad-scale haplotype inference are restricted primarily to coding regions^{5,6,14-16,27.} To fully characterize the IG loci at the genome and population level, specialized genotyping methods capable of capturing locus-wide polymorphism at nucleotide resolution are required. Indeed, such methods have been applied elsewhere in the genome to resolve complex and hyper-polymorphic loci, including other loci in the immune system^{28,29}.

Long-read sequencing technologies have been shown to resolve complex regions such as killer immunoglobulin-like receptors (KIR)^{30,31}, human leukocyte antigen (HLA)^{32,33} and chromosomal rearrangements³³, identify novel SVs^{34,35}, and identify SVs missed by standard short-read sequencing methods^{36,37}. Additionally, it has now been shown that the sensitivity of SV detection can be improved by attempting to resolve variants in a haplotype-specific manner^{37,38}. When long-read sequencing is combined with methods to specifically target a genomic locus, either with a CRISPR/Cas9 system^{39,40} or DNA probes^{41,42}, it has been shown to effectively resolve such regions. Targeted approaches have also enabled a higher resolution of HLA typing⁴³ and KIR typing^{44,45}.

Here, we present a new framework that leverages target-enrichment-based long-read sequencing, paired with a new IG genomics analysis tool, IGentoyper, to comprehensively characterize germline variation in the IGH locus. We demonstrate the utility of this strategy by applying it to genomic DNA from 9 human samples, including a haploid hydatidiform mole cell line, two mother-father-child trios, and two additional unrelated individuals. Using orthogonal data and pedigree information available for several of these samples for benchmarking and validation, we show that the application of our approach leads to high-quality haplotype-specific assemblies across the IGH locus, allowing for the comprehensive detection and genotyping of SNVs, insertions and deletions (indels) (1-50 bps), SVs, as well as annotation of IG gene segments, alleles, and associated non-coding elements. In addition, we show that the additional use of long-range phasing/haplotype information (e.g., parental genotypes) improves assembly contiguity across the locus. Data from multiple Pacific Biosciences platforms and chemistries can be used, and the integration of highly accurate long circular consensus sequencing (CCS) reads offers improved performance and internal validation of variants characterized from subread-based assemblies. We provide data on sample multiplexing in a single SMRTcell, showing that this strategy results in comparable sequencing assembly and genotype metrics, providing evidence that our approach can be scaled in a cost-effective manner, without impacting data quality. Finally, we show that our genotype call sets have improved accuracy over existing datasets generated using alternative short-read and array-based methods. Our strategy represents a critical step towards the complete ascertainment of IG germline genetic variation, a requirement for bettering our understanding of the genetic basis of Ab-mediated processes in human disease and clinical phenotypes⁴⁶.

Novel Tools for Comprehensively Characterizing IG Haplotype Diversity

The application of long-read sequencing technologies has been shown to resolve extremely complex loci^{32,34,47}. Most applications have primarily used whole-genome sequencing data. However, at present performing whole-genome long-read sequencing on large collections of samples is neither cost-effective nor high-throughput⁴⁸. To circumvent these barriers, and establish a framework for interrogating locus-wide IGH variants, we implemented an approach that pairs target-enrichment DNA capture with Pacific Biosciences (PacBio) long-read sequencing.

We tested two different custom Roche Nimblegen SeqCap EZ target-enrichment panels, each designed using DNA target sequences from the human IGH locus. Critically, rather than using only a single representative IGH haplotype—for example, those available as part of either the hg19 or GRCh38 human reference assembly—we based our design off of non-redundant sequences from the GRCh38 haplotype³, as well as all additional complex SV and insertion haplotypes known to harbour sequences not present in GRCh38^{3,49}. For one design (referred to as “panel A”), targets were focused to sequences spanning the IGHJ, IGHD, and IGHV gene regions; in the second design (referred to as “panel B”), the same targets were used, but additional targets in the IGHC gene region of GRCh38 were also included. IGHC-related sequences are not considered in the current iteration of our analysis pipeline, but IGHC analysis features are currently under development.

To process and analyze long-read IGH genomic sequencing data, we developed a new informatics tool, IGenotyper. IGenotyper utilizes and builds on existing assembly tools to map and phase PacBio long reads and generate diploid assemblies across the IGH locus, leading to summary reports that consist of comprehensive SNV, indel, and SV genotype call sets, as well as IGHV, IGHD, and IGHJ gene/allele annotations. For read mapping, SNV/indel/SV calling, and sequence annotation, the current pipeline leverages a custom IGH locus genomic reference that represents known SV variant haplotype sequences in a contiguous, non-redundant fashion; this locus reference harbors the same sequence targets used for the design of target-enrichment panels, and ensures that known SVs circulating in the human population are effectively interrogated.

Benchmarking Performance Using a Haploid DNA Sample

To initially benchmark the performance of our approach, we used genomic DNA from a haploid hydatidiform mole sample (CHM1), from which we had previously assembled the IGHJ, IGHD, and IGHV gene segment regions from Bacterial Artificial Chromosome (BAC) clones using Sanger sequencing³; these BAC based assemblies are now the representation of IGH in the current GRCh38 reference genome build. Using both of the IGH capture panel designs mentioned above, we prepared SMRTbell libraries (5-8 Kb) for sequencing on either the RSII or Sequel 1 platforms. After mapping sequence reads from each library to our custom reference, we observed an on-target rate (i.e., fraction of reads mapping to intended IGH targets compared to the rest of the genome) of 31.8% and 47.6% for the RSII and Sequel 1 datasets, respectively. This equated to a mean subread coverage across the IGHV, IGHD, and IGHJ regions collectively of 557.9×(RSII) and 12006.4×(Sequel 1), and mean circular consensus sequence (CCS) read coverage of 45.1×(RSII) and 778.2×(Sequel 1). The average phred quality score of the CCS reads from the Sequel 1 library was 70.27 (99.999991% accurate), with an average read length of 6,457.06 bp. We have designed IGentoyper to utilize both the subreads and the higher quality CCS reads, for optimal coverage and assembly performance; the option to use either subreads only, or both subreads and CCS reads can be decided based on the experimental setup. We noted one major difference between the read coverage profiles of the two target-enrichment panels tested (A and B). While the mean coverage was consistent in panel B for the IGHV, IGHD, and IGHJ regions, we noted a stark loss in coverage over the IGHJ region in panel A. We speculate this is caused by a lack of adjacent target sequence on the 3′ flank of the IGHJ region in panel A, in contrast to panel B, which also included sequence targets across the entirety of the IGHC region.

To most effectively use these data to benchmark the performance of IGenotyper, we combined reads from both libraries to mitigate inconsistencies in regional coverage caused by differences in the target-enrichment panels. Based on this combined dataset, we determined that 970,302 bp (94.8%) of the IGHV, IGHD, and IGHJ regions (chr14:105859947-106883171) were spanned by >1000 subreads. Likewise, 1,006,287 bp (98.3%) were spanned by >20 CCS reads. With respect to IGHV, IGHD, and IGHJ coding sequences, specifically, the mean CCS coverage was 160.3×(median=42.5×).

We next determined whether the IGHV, IGHD, and IGHJ gene regions could be assembled using the target-enrichment-based long-read sequencing data. CHM1 has been previously Sanger-sequenced and assembled from large-insert clones³, and serves as the IGH locus in the human reference build GRCh38. We used this orthogonal dataset to determine how much of the IGH locus can be assembled using our approach and assess the accuracy of the assembly. Using the combined read dataset, IGenotyper assembled 1,005,764 bases (98.3%) of the IGH locus, represented by 95 contigs. Of the 1,005,764 bp that were assembled, only 184 single nucleotide differences were observed compared to GRCh38 (<0.0002% of bases), amounting to a base pair concordance >99.9%. The majority of discordant bases(109/184) were found in just 4.2% (4/95) of the assembled contigs, and were localized to regions totaling XX bp of the assembly, all of which were associated with complex repeat/duplication sequences within the locus; in most cases it is difficult to discern whether the discrepancies arise due to errors in the Sanger or PacBio-based assemblies. Nonetheless, the small number of discordant bases and their concentrated location in complex sequence demonstrates that the overall IGenotyper assembly is highly accurate.

All known SV regions that had been previously described in CHM1³ were also captured in this dataset, and thus the assembly accounted for all IGHV (n=6), IGHD (n=27), and IGHJ (n=47) gene segments in this sample. In addition to genes previously characterized by BAC sequencing, the IGentoyper assembly also spanned IGHV7-81; however, because this gene did not have corresponding BAC assemblies we excluded from the current analysis. When we compared allele calls at IGH gene segments made by IGentoyper (FIG. 2X), we observed 100% concordance to those that had been identified previously by Sanger sequencing³.

Assessing the Accuracy of Diploid Assemblies in the IGH Locus

We next determined the accuracy of haplotype-specific assemblies in diploid samples. Previous studies have demonstrated that assembling diploid genomes in a haplotype-specific manner increases the accuracy of variant detection^{36-38,50-53}. For benchmarking purposes, we focused again on samples with available orthogonal assembly data and variant call sets. One of the most valuable resources for such samples is the 1000 Genomes Project⁵⁴ (1KGP), which includes many samples that have been extensively sequenced/characterized using myriad technologies, and in some cases familial samples can be obtained. Targeted sequencing of large-insert clones in the IGH region has also been conducted in a small subset of these individuals³. To take advantage of these existing datasets, we selected one trio and one individual sample from the 1KGP to assess the performance of our approach in diploid samples (maybe point to supp table?). The trio was of African ancestry from the Yoruban (YRI) population (NA19240, NA19238, NA1239), and the individual sample was of European ancestry from the CEPH population (NA12878). Because 1KGP samples are derived from lymphoblastoid cell lines and are thus known to harbor rearrangements within the IG loci³, we focused our analysis of these samples on the IGHV region. IGH target-enrichment was performed on these samples using panel A and sequenced on either the RSII or Sequel 1 platforms (see Supplementary Table 1 for details). Resulting datasets were then analyzed using IGenotyper (FIG. 1). For diploid samples, IGenotyper first identifies haplotype blocks using all CCS reads that span multiple heterozygous SNVs within a sample. Within each haplotype block, CCS reads are then partitioned into their respective haplotype, and are then assembled independently to derive assembly contigs representing each haplotype in that individual. Reads spanning blocks of homozygosity that cannot be phased with flanking heterozygous positions are assembled using all the reads within those regions, as these blocks are considered to represent either: 1) homozygous regions, in which both haplotypes in the individual are presumed to be identical, or 2) hemizygous regions, in which the individual is presumed to harbor either an insertion or deletion only on one chromosome (Supplementary Figure X).

We assessed performance using data from the proband, NA19240, of the selected trio and NA12878. IGenotyper assemblies were composed of 51 and 41 haplotype blocks in NA19240 and NA12878, respectively. Of these, 25/51 and 20/41 in each respective sample were identified as heterozygous, in which haplotype-specific assemblies could be generated, totaling 773,748 bp (64.85%) in NA19240, and 486,101 bp (40.74%) in NA12878. Within these heterozygous blocks, the mean number of heterozygous positions was 76.16 (NA19240) and 68.25 (NA12878), compared to a mean number of 1.9 and 1.3 heterozygous positions in homozygous blocks. Summing the bases assembled across both heterozygous and homozygous/hemizygous contigs in each sample, complete assemblies comprised 2.3 Mb of diploid resolved sequence in NA19240 and 1.9 Mb in NA12878. Including all known insertion/SV haplotypes, a complete diploid assembly of the IGH locus should be roughly ˜2.4 Mb.

We next validated the accuracy of NA19240 and NA12878 assemblies using several orthogonal datasets: Sanger-sequenced fosmids (n=6, NA19240; n=2, NA12878), paired-end Illumina data, and previously assembled chromosome-level assemblies generated by the Reference Genome Improvement Consortium (RGI). The Sanger-sequenced fosmids spanned 240,485 bps of the NA19240 assembly and 74,803 bps of the NA12878 assembly. The percent identity between the Sanger-sequenced fosmids and the corresponding assembled contigs was 99.98% for both NA19240 and NA12878. In order to assess the accuracy of the whole assembly, paired-end Illumina data from NA19240 and NA12878 was aligned to each assembly. Pilon, an assembly error-correction tool, was used to read the alignment of the paired-end Illumina data to the assembly and detect errors. A total of 77 bp errors and 102 gap errors across the 2.3 Mb NA19240 assembly, and 125 bp errors and 167 gap errors across the 1.9 Mb NA12878 was found. Using the paired-end data as an evaluation method gives these assemblies an accuracy of 99.996% and 99.991%. In order to evaluate the assembly approach and further evaluate the accuracy of the assembly, the IGenotyper assembly was aligned to previously generated chromosome-level assemblies by the RGI Consortium. The RGI assemblies represent only a single haplotype from these individuals and were, assembled using high coverage whole genome PacBio sequence and BioNano data, and error-corrected with Illumina data. IGenotyper contigs corresponding to the same RGI selected haplotype were identified and aligned to the RGI chromosome-level assembly. The NA19240 IGenotyper assembly spanned 941,955/999,979 (94.2%) bp of the RGI assembly, and NA12878 spanned 726,172/738,672 (98.3%) bp of the RGI assembly. Both of the RGI assemblies were shorter than those produced by IGenotyper, but between the RGI and IGenotyper assemblies, there was an overlap of 969,394/1,007,245 bp (96.2%) and 777,521/788,480 (98.6%) bp for NA19240 and NA12878, respectively. Fewer bases were compared in the NA12878 RGI assembly because the chromosome-level assembly contained a V(D)J recombination event. Between the two NA19240 assemblies 1,19819 bases were discordant; 56195 base mismatches were observed between the NA12878 assemblies. CCS reads were used to assess support for bases identified in each assembly at these discordant positions. CCS reads supported the nucleotides found in the IGenotyper assemblies for 9978/1,19819 bases in NA19240 and 51650/56195 bases in NA12878. Several errors in the RGI assembly were due to mixing of haplotypes (Supplementary figure). Taking into account the differences found to be errors in the NA19240 and NA12878 IGenotyper assembly, the accuracy for each was 99.987% and 99.99%. These errors do not propagate into the variant call set, as each variant is validated using the highly accurate CCS reads. Together, these multiple levels of orthogonal validation show that the target-enrichment-based long-read sequencing data, paired with IGenotyper, can be used to accurately assemble IGH from a diploid sample.

Assessing Local Phasing Accuracy and Extending Haplotype-Specific Assemblies with Long-Range Phasing Information

We next assessed the local phasing accuracy of haplotype blocks in NA19240 and NA12878. When run with standard parameters, IGentoyper will use read-back phasing to identify reads from the same haplotype and delineate haplotype blocks within an individual, prior to assembly. Here we can test the accuracy of local phasing (correct phase of genotypes within each contig/haplotype block) by comparing read-back phased genotypes in these samples to trio-based phased genotypes, leveraging data from the parents of NA19240. To ensure the reliability of this test, we considered only parental genotypes with high CCS coverage. No phase-switch errors were observed in any of the heterozygous haplotype blocks (n=253 blocks, NA19240). Within homozygous blocks, bases genotypes did not follow a mendelian inheritance pattern. This suggests that the individual contig assemblies generated by IGenotyper within heterozygous blocks have high phasing accuracy.

In both NA19240 and NA12878, we observe low localized read coverage (dropout) in various regions of the locus within an individual sample, representing technical limitations of DNA capture. Because of this and regions of homozygosity/hemizygosity, IGenotyper is limited in its ability to generate fully phased haplotype assemblies across the entirety of the locus. However, we reasoned that when long-range phase information is also available (e.g., trio-based phased genotypes) all contigs from an IGenotyper assembly could be correctly assigned to each parental haplotype and phased accordingly. To assess this, heterozygous SNVs from NA19240 were phased using both long sequencing reads and parental SNVs. This reduced the number of haplotype blocks from 25 to 1. NA19240 was assembled again to determine the effect of assembling a completely phased IGH locus versus locally phased. Only 2 base differences were found between the locally phased and long-range phased assemblies, indicating that, while assemblies generated in the absence of long-range phased variant data are not less accurate on the whole, use of long-range phasing information can improve overall assembly contiguity, which ultimately may more effectively aid in the study of long-range genetic/haplotype effects.

Without wishing to be bound by theory, alternative forms of long-range phasing data can also be available for a sample of interest. For example, because V(D)J recombination uses a single chromosome to generate an antibody, allelic variants within IGHV, IGHD, and IGHJ can also be phased using expressed AIRR-seq data (14,15). Although AIRR-seq data is not available for NA19240 and NA12878, we are able to crudely assess whether AIRR-seq based haplotype inference could also help improve contig phasing in IGenotyper assemblies, by identifying the number of heterozygous haplotype blocks with heterozygous IGHV gene segments. This highlights one potential strength of pairing these complementary data types to larger numbers of samples.

Accurate Assemblies Result in Comprehensive and Accurate Variant Call Sets

The construction of diploid assemblies facilitates greater resolution of the full spectrum of genetic variant classes⁵⁵. In addition to IGH locus assembly, IGenotyper can be used to detect SNVs, short indels, SVs including genotypes for eight known large polymorphic SVs (9-75 Kb) and their associated SNVs. To the best of our knowledge, this is the first tool that can comprehensively genotype all different variant types across the IGH locus. To demonstrate this, we assessed the concordance of proband (NA19240) and parental variant call sets, and determined that the overwhelming majority of variants were consistent with mendelian inheritance. Across the IGHV region we identified 2,391 SNVs, 18670 short indels (1-49 bps) (8833 deletions; 9837 insertions), and 16XX SVs (>50 bps) in NA12940. Collectively, IGenotyper-based genotypes for the parents of NA19240 supported 2,312/2,391 SNVs, 7229/8733 deletions and 8731/9737 insertions, and 16X/16X SVs in NA19240. 23 unsupported indels (14 deletions and 9 insertions) were 1 bp indels, and 2 unsupported indels were 2 and 3 bps. These are mostly like assembly errors. However, they only represent a small proportion of the assembly and of the total identified variants (0.88% of variants).

A critical component of our approach is the use of a modified reference assembly that incorporates sequence of known SVs accounting for insertion sequence not present in either GRCh37 or GRCh38. For example, a ˜61.1 Kb insertion with containing the genes IGHV4-38-2, IGHV3-34D, IGHV3-38-3 and IGHV 1-38-4 is not present in either GRCh37 or GRCh38. Thus, variant detection pipelines aligning that align reads to GRCh37 or GRCh38 would miss variants coming from this insertion sequence. Use of our modified reference allows not only for the detection of these SVs and, but also SNVs and /indels within these SVs. Specifically, our modified reference also contains four insertion/complex SVs, which were integrated into the GRCh38 IGH locus assembly.

Next, the accuracy of indel detection was tested using the trio. 84/108 deletions (1-50 bps) found in NA19240 were present in at least one parent. The 24 deletions not found in the parents were 1 bp deletions. A 21 bp insertion not found in the parent was validated by CCS reads and might represent a de novo insertion. 7 insertions not found in the parents were 1 bp insertions and 1 insertion not found was a 2 bp insertion.

Sample Multiplexing Leads to Reproducible Assemblies and Variant Call Sets

Running a single sample on a SMRT cell gives extremely high CCS coverage. Without wishing to be bound by theory, sequencing multiple samples on a single SMRT cell will still effectively capture IGH. 4 replicates of NA12878 were multiplexed on a single SMRT cell. The average subread coverage and CCS coverage per sample was 655.3× and 73.81×. The max CCS coverage difference between replicates was 1.15×. Each replicate was put through IGenotyper. Each replicate assembly was compared to each other. In order to compare the assemblies, one replicate assembly was labelled as reference and the other replicate assembly was labelled as query. The query was aligned to the reference replicate assembly. Across all the comparisons, 99.64% of the reference was completely spanned. These regions were completely spanned with 100% sequence identity.

SNVs, indels and SVs were also compared across replicates. An average of 2852 SNVs were found across the replicates. 2772 SNVs overlapped all replicates. An average of 10.5 unique SNVs per sample was found. Likewise, an average of 168 indels are present across the replicates. 129 indels overlapped all replicates. An average of 15.25 unique indels per sample was found.

Given the extremely high CCS coverage using Sequel, we can still effectively capture the IGH locus by multiplexing samples on a single SMRT cell. 4 samples were multiplexed on a single SMRT cell. This reduced the IGH subread coverage to ˜655× and IGH CCS coverage to ˜73.5×. This also reduces the price range to sequence the IGH locus and allows this method to be used in larger cohorts. Importantly, multiplexing the same sample showed similar sequencing statistics.

Identifying False-Negative and -Positive IGH Variants in Public Datasets

We next sought to place our IGenotyper variant call sets in the context of publically available datasets previously generated in the same samples, such as those generated from the 1KG project using short-read data alone, or combinations of short and long-reads paired with additional technologies. Pitfalls of using short-read data for IGH variant detection and gene segment annotation have been discussed previously (Watson and Breden 2012; Watson et al. 2017 JI letter to editor). Given that we have extensively vetted the IGenotyper assemblies and variant call sets for CHM1, NA19240, and NA12878, resulting in high-quality genotypes across IGH, we wanted to assess the advantages of our approach compared to alternatives.

First, for CHM1, we generated a benchmarking ground truth SNV dataset by aligning the IGH locus haplotype from GRCh38 (Watson et al., 2013) to that of GRCh37 (Matsuda et al. 1998). This resulted in the identification of 2,940 SNVs between these two haplotypes. To generate comparable datasets, we next aligned an available Illumina paired-end sequencing dataset generated from CHM1 (ref), as well as our CHM1 IGenotyper assemblies to the GRCh37 IGH haplotype. We detected 4,433 IGH SNVs in the Illumina dataset, and 2,958 SNVs in the IGenotyper assembly. Comparing these to the benchmarking dataset (i.e., GRCh38 aligned to GRCh37), the Illumina call set included only 73.2% (2,153) of the ground truth SNVs, and also included an additional 2,274 false-positive SNVs. Using the IGenotyper CHM1 assembly, 99.0% (2,912) of the ground truth SNVs were detected, and only 46 (1.56%) false-positive SNVs were called.

We next compared SNVs genotyped by IGenotyper in NA19240 and NA12878 to those available in the 1KGP Phase 3 dataset.

The NA19240 indels were also compared the indels identified by the 1000 Genome Structural Variation Consortium using a combination of WGS Illumina and PacBio data with several different algorithms. All 22 indels from 4-50 bps detected by 1000 Genome Structural Variation Consortium were detected. An additional 24 indels not identified by the 1000 Genome Structural Variation Consortium were also detected.

In addition to SNVs and indel calling, SVs are also detected and a set of 11 SVs (6 unique SVs and 5 different haplotypes from a single polymorphic SV) are directly genotyped using phased CCS reads and assembly. One SV contains 5 different haplotypes3 and so by using the genotype of the IGHV genes present in those 5 different haplotypes, we can further try to determine which haplotype is present in a sample as opposed to just determining the presence of an alternate haplotype.

7 deletions less than 400 bps and 3 large deletions (˜9.5 Kb, ˜38 Kb, ˜46 Kb) were detected in NA19240. 6/7 deletions less than 400 bps were found in the 1000 Genome Structural Variation Consortium SV dataset. The missed deletion was validated by parental data. All 3 large deletions were not in the 1000 Genome Structural Variation Consortium SV dataset. The largest detected deletion (˜46 Kb) was validated with BioNano data. 3 deletions less than 1 Kb in the 1000 Genome Structural Variation Consortium SV dataset that did not overlap detected deletions by IGenotyper overlapped a complex SV detected by IGenotyper.

7 insertion less than 500 bps and 4 large insertions (˜61 Kb, ˜10.8 Kb, ˜37.7 Kb, ˜49.2 Kb) were detected in NA19240. 7 insertions less than 500 bps in NA19240 were found in the 1000 Genome Structural Variation Consortium SV dataset. Additionally, 4 large insertions (˜61 Kb, ˜10.8 Kb, ˜37.7 Kb, ˜49.2 Kb) were genotyped. The largest insertion (˜61 Kb) has been previously detected in this sample using large-insert clones3. The ˜10.8 Kb insertion and a portion of ˜61 Kb insertion was found in the 1000 Genome Structural Variation Consortium SV dataset. All 4 large insertions were validated with the parental data and 3/4 insertions were validated with BioNano data. 4/17 insertions (<170 bps) found in the 1000 Genome Structural Variation Consortium SV dataset were not detected. No evidence in the parental or probands CCS data were found for 3/4 insertions. 1 insertion was not detected due to decreased coverage in the region.

Effect of False and Missed Variants on Imputation

The 1KGP Phase 3 SNV call sets are widely used for imputing SNVs in GWAS. In order to determine the effect of inaccurate SNVs and missing SNVs within 1KGP Phase 3 dataset, we compared previously imputed SNVs²⁰ within the IGH locus to SNVs detected with IGenotyper in a RHD sample. This sample was initial genotyped, and imputed using SHAPEIT. Of the 1,034 SNVs in this GWAS-based data for the sample, 521 SNVs were correctly imputed and 513 SNVs were incorrectly imputed. In addition, IGenotyper detected an additional 2,562 SNVs that were not assayed in this sample previously.

Demonstrating the Utility of Igenotyper for IGH Gene Segment Curation and Characterization of Haplotype Diversity

In addition to generating highly accurate assemblies and variant call sets, IGenotyper provides additional output in the form of several summary files, including a sample summary report, with assembly overview metrics, as well as basic variant annotationIG gene segment/allele calls, and basic variant annotation (e.g., intergenic, coding, and IG gene segment allele calls)

### REFERENCES CITED IN THIS EXAMPLE

1. Lefranc, M.-P. & -P. Lefranc, M. IMGT, the international ImMunoGeneTics database. Nucleic Acids Research 29, 207-209 (2001).
2. Boyd, S. D. et al. Individual variation in the germline Ig gene repertoire inferred from variable region gene rearrangements. J. Immunol. 184, 6986-6992 (2010).
3. Watson, C. T. et al. Complete Haplotype Sequence of the Human Immunoglobulin Heavy-Chain Variable, Diversity, and Joining Genes and Characterization of Allelic and Copy-Number Variation. The American Journal of Human Genetics 92, 530-546 (2013).
4. Gadala-Maria, D., Yaari, G., Uduman, M. & Kleinstein, S. H. Automated analysis of high-throughput B-cell sequencing data reveals a high frequency of novel immunoglobulin V gene segment alleles. Proc. Natl. Acad. Sci. U.S.A. 112, E862-70 (2015).
5. Scheepers, C. et al. Ability To Develop Broadly Neutralizing HIV-1 Antibodies Is Not Restricted by the Germline Ig Gene Repertoire. The Journal of Immunology 194, 4371-4378 (2015).
6. Corcoran, M. M. et al. Production of individualized V gene databases reveals high levels of immunoglobulin genetic diversity. Nat. Commun. 7,13642 (2016).
7. Thornqvist, L. & Ohlin, M. The functional 3′-end of immunoglobulin heavy chain variable (IGHV) genes. Mol. Immunol. 96, 61-68 (2018).
8. Calonga-Solis, V. et al. Unveiling the Diversity of Immunoglobulin Heavy Constant Gamma (IGHG) Gene Segments in Brazilian Populations Reveals 28 Novel Alleles and Evidence of Gene Conversion and Natural Selection. Frontiers in Immunology 10, (2019).
9. Milner, E. C., Hufnagle, W. O., Glas, A. M., Suzuki, I. & Alexander, C. Polymorphism and utilization of human VH Genes. Ann. N. Y. Acad. Sci. 764, 50-61 (1995).
10. Sasso, E. H., Johnson, T. & Kipps, T. J. Expression of the immunoglobulin VH gene 51p1 is proportional to its germline gene copy number. J. Clin. Invest. 97, 2074-2080 (1996).
11. Chimge, N.-O. et al. Determination of gene organization in the human IGHV region on single chromosomes. Genes Immun. 6, 186-193 (2005).
12. Pramanik, S. et al. Segmental duplication as one of the driving forces underlying the diversity of the human immunoglobulin heavy chain variable gene region. BMC Genomics 12, 78(2011).
13. Kidd, M. J., Jackson, K. J. L., Boyd, S. D. & Collins, A. M. DJ Pairing during VDJ Recombination Shows Positional Biases That Vary among Individuals with Differing IGHD Locus Immunogenotypes. J. Immunol. 196, 1158-1164 (2016).
14. Kidd, M. J. et al. The inference of phased haplotypes for the immunoglobulin H chain V region gene loci by analysis of VDJ gene rearrangements. J. Immunol. 188, 1333-1340 (2012).
15. Gidoni, M. et al. Mosaic deletion patterns of the human antibody heavy chain gene locus shown by Bayesian haplotyping. Nat. Commun. 10, 628 (2019).
16. Luo, S., Yu, J. A., Li, H. & Song, Y. S. Worldwide genetic variation of the IGHV and TRBV immune receptor gene families in humans. Life Sci Alliance 2, (2019).
17. Avnir, Y. et al. IGHV1-69 polymorphism modulates anti-influenza antibody repertoires, correlates with IGHV utilization shifts and varies by ethnicity. Sci. Rep. 6, 20842 (2016).
18. Watson, C. T. & Breden, F. The immunoglobulin heavy chain locus: genetic variation, missing data, and implications for human disease. Genes Immun. 13, 363-373 (2012).
19. Watson, C. T., Glanville, J. & Marasco, W. A. The Individual and Population Genetics of Antibody Immunity. Trends in Immunology 38, 459-470 (2017).
20. Parks, T. et al. Association between a common immunoglobulin heavy chain allele and rheumatic heart disease risk in Oceania. Nat. Commun. 8, 14946 (2017).
21. Witoelar, A. et al. Meta-analysis of Alzheimer's disease on 9,751 samples from Norway and IGAP study identifies four risk loci. Scientific Reports 8, (2018).
22. Glanville, J. et al. Naive antibody gene-segment frequencies are heritable and unaltered by chronic lymphocyte ablation. Proceedings of the National Academy of Sciences 108, 20066-20071 (2011).
23. Wang, C. et al. B-cell repertoire responses to varicella-zoster vaccination in human identical twins. Proc. Natl. Acad. Sci. U.S.A. 112, 500-505 (2015).
24. Rubelt, F. et al. Individual heritable differences result in unique cell lymphocyte receptor repertoires of naïve and antigen-experienced cells. Nat. Commun. 7,11112 (2016).
25. Greiff, V. et al. Systems Analysis Reveals High Genetic and Antigen-Driven Predetermination of Antibody Repertoires throughout B Cell Development. Cell Rep. 19, 1467-1478 (2017).
26. Kidd, J. M. et al. A human genome structural variation sequencing resource reveals insights into mutational mechanisms. Cell 143, 837-847 (2010).
27. Luo, S., Yu, J. A. & Song, Y. S. Estimating Copy Number and Allelic Variation at the Immunoglobulin Heavy Chain Locus Using Short Reads. PLoS Comput. Biol. 12, e 1005117 (2016).
28. Norman, P. J. et al. Defining KIR and HLA Class I Genotypes at Highest Resolution via High-Throughput Sequencing. The American Journal of Human Genetics 99, 375-391 (2016).
29. Neville, M. J. et al. High resolution HLA haplotyping by imputation for a British population bioresource. Hum. Immunol. 78, 242-251 (2017).
30. Roe, D. et al. Revealing complete complex KIR haplotypes phased by long-read sequencing technology. Genes Immun. 18, 127-134 (2017).
31. Suzuki, S. et al. Reference Grade Characterization of Polymorphisms in Full-Length HLA Class I and II Genes With Short-Read Sequencing on the ION PGM System and Long-Reads Generated by Single Molecule, Real-Time Sequencing on the PacBio Platform. Frontiers in Immunology 9, (2018).
32. Wenger, A. M. et al. Accurate circular consensus long-read sequencing improves variant detection and assembly of a human genome. Nat. Biotechnol. (2019). doi:10.1038/s41587-019-0217-9
33. Cretu Stancu, M. et al. Mapping and phasing of structural variation in patient genomes using nanopore sequencing. Nat. Commun. 8, 1326 (2017).
34. Chaisson, M. J. P. et al. Resolving the complexity of the human genome using single-molecule sequencing. Nature 517, 608-611 (2015).
35. Audano, P. A. et al. Characterizing the Major Structural Variant Alleles of the Human Genome. Cell 176, 663-675.e19 (2019).
36. Chaisson, M. J. P. et al. Multi-platform discovery of haplotype-resolved structural variation in human genomes. Nat. Commun. 10, 1784 (2019).
37. Huddleston, J. et al. Discovery and genotyping of structural variation from long-read haploid genome sequence data. Genome Res. 27, 677-685 (2017).
38. Pendleton, M. et al. Assembly and diploid architecture of an individual human genome via single-molecule technologies. Nat. Methods 12, 780-786 (2015).
39. Hafford-Tear, N. J. et al. CRISPR/Cas9-targeted enrichment and long-read sequencing of the Fuchs endothelial corneal dystrophy-associated TCF4 triplet repeat. Genetics in Medicine 21, 2092-2102 (2019).
40. Ebbert, M. T. W. et al. Long-read sequencing across the C9orf72 ‘GGGGCC’ repeat expansion: implications for clinical use and genetic discovery efforts in human disease. Mol. Neurodegener. 13, 46 (2018).
41. Hoff, S. N. K. et al. Long-read sequence capture of the haemoglobin gene clusters across codfish species. Mol. Ecol. Resour. 19, 245-259 (2019).
42. Bethune, K. et al. Long-fragment targeted capture for long-read sequencing of plastomes. Applications in Plant Sciences 7, e1243 (2019).
43. Mayor, N. P. et al. HLA Typing for the Next Generation. PLoS One 10, e0127153 (2015).
44. Bultitude, W. P., Gymer, A. W., Robinson, J., Mayor, N. P. & Marsh, S. G. E. KIR2DL1 allele sequence extensions and discovery of 2DL1*0010102 and 2DL1*0010103 alleles by DNA sequencing. Hladnikia 91, 546-547 (2018).
45. Turner, T. R. et al. Single molecule real-time DNA sequencing of HLA genes at ultra-high resolution from 126 International HLA and Immunogenetics Workshop cell lines. Hladnikia 91, 88-101 (2018).
46. Huddleston, J. & Eichler, E. E. An Incomplete Understanding of Human Genetic Variation. Genetics 202, 1251-1254 (2016).
47. Jain, M. et al. Nanopore sequencing and assembly of a human genome with ultra-long reads. Nat. Biotechnol. 36, 338-345 (2018).
48. Mitsuhashi, S. & Matsumoto, N. Long-read sequencing for rare human genetic diseases. J. Hum. Genet. (2019). doi:10.1038/s10038-019-0671-8
49. Matsuda, F. et al. The Complete Nucleotide Sequence of the Human Immunoglobulin Heavy Chain Variable Region Locus. The Journal of Experimental Medicine 188, 2151-2162 (1998).
50. Koren, S. et al. De novo assembly of haplotype-resolved genomes with trio binning. Nat. Biotechnol. (2018). doi:10.1038/nbt.4277
51. Rodriguez, O. L., Ritz, A., Sharp, A. J. & Bashir, A. MsPAC: A tool for haplotype-phased structural variant detection. Bioinformatics (2019). doi:10.1093/bioinformatics/btz618
52. Chin, C.-S. et al. Phased diploid genome assembly with single-molecule real-time sequencing. Nat. Methods 13, 1050-1054 (2016).
53. Weisenfeld, N. I., Kumar, V., Shah, P., Church, D. M. & Jaffe, D. B. Direct determination of diploid genome sequences. Genome Res. (2017). doi:10.1101/gr.214874.116
54. 1000 Genomes Project Consortium et al. A global reference for human genetic variation. Nature 526, 68-74 (2015).
55. Chaisson, M. J. P., Wilson, R. K. & Eichler, E. E. Genetic variation and the de novo assembly of human genomes. Nat. Rev. Genet. 16, 627-640 (2015).
56. Church, D. M. et al. Extending reference assembly models. Genome Biol. 16, 13 (2015).
57. Chaudhary, N. & Wesemann, D. R. Analyzing Immunoglobulin Repertoires. Front. Immunol. 9, 462 (2018).
58. Martin, M. et al. WhatsHap: fast and accurate read-based phasing. bioRxiv 085050 (2016). doi:10.1101/085050
59. Koren, S. et al. Canu: scalable and accurate long-read assembly via adaptive k-mer weighting and repeat separation. Genome Res. 27, 722-736 (2017).

### Equivalents

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method of preparing a vaccine composition specific to a subject with a genotype responsive to the vaccine composition, comprising the steps of:
obtaining a biological sample from the subject;
identifying germ-line polymorphisms at a immunoglobulin (IG) loci in the tissue sample;
identifying antibody repertoire in the tissue sample;
comparing the germ-line polymorphisms to the antibody repertoire to identify the subject as responsive to a vaccine composition; and
preparing a vaccine composition specific for the subject.

2. A method of vaccinating a subject, the method comprising the steps of:
obtaining a biological sample from the subject;
identifying germ-line polymorphisms at a immunoglobulin (IG) loci in the tissue sample;
identifying antibody repertoire in the tissue sample;
comparing the germ-line polymorphisms to the antibody repertoire to identify the subject as responsive to a vaccine composition; and
administering the vaccine composition to the subject.

3. A method of identifying a subject as responsive to a vaccine composition, comprising the steps of:
obtaining a biological sample from the subject;
identifying germ-line polymorphisms at a immunoglobulin (IG) loci in the tissue sample;
comparing the germ-line polymorphisms in the tissue sample to known germ-line polymorphisms, wherein the known germ-line polymorphisms are indicative of responsiveness to the vaccine composition; and
identifying the subject as responsive to the vaccine composition if the subject's germ-line polymorphisms are similar to the known germ-line polymorphisms.

4. A method of vaccine discovery, the method comprising the steps of:
obtaining biological samples from a population of subjects;
identifying germ-line polymorphisms at a immunoglobulin (IG) loci in the tissue samples;
identifying the antibody repertoire in the tissue samples;
comparing the germ-line polymorphisms to the antibody repertoires to identify a population as responsive to a vaccine composition.

5. The method of claim 1-4, wherein the immunoglobulin loci comprises an immunoglobulin heavy chain loci, an immunoglobulin light chain loci, or both.

6. The method of claim 3, wherein the comparing step further comprises evaluating antibody convergence groups.

7. The method of claim 3, further comprising the step of administering the vaccine composition to the population of subjects.

8. The method of any one of claims 1-4, wherein the vaccine composition comprises an anti-influenza vaccine composition.

9. The method of any one of claims 1-5, wherein identifying germ-line polymorphisms comprises long-read sequencing of genomic DNA isolated from the biological sample.

10. The method of any one of claims 1-5, wherein identifying the antibody repertoire comprises sequencing cDNA generated from the tissue sample.

11. The method of any one of claims 1-5, wherein the antibody repertoire comprises a naïve antibody repertoire or a stimulated antibody repertoire.

12. The method of claims 5, wherein the IGH loci comprises the IGHD, IGHC, IGHV, or a combination thereof.

13. The method of claim 5, wherein the IG light chain loci comprises the IG lambda loci or the IG kappa loci.

14. The method of claim 5, wherein the IGH loci comprises the IGHV1-69 loci.

15. The method of any one of claims 1-5, wherein the vaccine comprises an influenza vaccine composition.

16. The method of any one of claims 1-3, wherein the subject comprises a population of subjects.
